(19) United States
(12) Patent Application Publication
(10) Pub. No.: US 2008/0112895 A1
(43) Pub. Date: May 15, 2008

(54) Aqueous dronabinol formulations

(75) Inventors: S. George Kottayil, Long Grove, IL (US); Venkat R. Goskonda, Phoenix, AZ (US); Zhongyuan Zhu, Vernon Hills, IL (US); Linet Kattookaran, Mount Prospect, IL (US)

Correspondence Address: Davidson, Davidson & Kappel, LLC, 485 7th Avenue, 14th Floor, New York, NY, 10018, US

(73) Assignee: Insys Therapeutics Inc., Phoenix, AZ (US)

(21) Appl. No.: 11/891,017

(22) Filed: Aug. 6, 2007

Related U.S. Application Data
(60) Provisional application No. 60/835,738, filed on Aug. 4, 2006.

(51) Int. Cl. A61K 31/353; A61K 36/185; A61K 9/14

(52) U.S. Cl. 424/46; 514/454; 424/774

(57) ABSTRACT

A room temperature stable aqueous cannabinoid formulation is disclosed. In preferred embodiments, the cannabinoid formulation comprises dronabinol in a mixture of buffer solution, and organic cosolvents such as ethanol, propylene glycol and polyethylene glycol.

This application claims the benefit of U.S. Provisional Patent Application No. 60/835,738, filed on Aug. 4, 2006, the disclosure of which is hereby incorporated by reference in its entirety.

### TECHNICAL FIELD

The present invention relates to formulations of cannabinoids containing water which are stable at room temperature for extended periods of time, e.g., for two years or more. The present invention is further related to aqueous cannabinoid formulations that are suitable for intrapulmonary delivery, oral delivery, sublingual delivery, transdermal delivery, intravenous delivery and ophthalmic delivery. The invention has utility in the fields of pharmaceutical formulation, pharmacology and medicine.

### BACKGROUND OF THE INVENTION

Delta-9-Tetrahydrocannabinol (also known as THC, dronabinol and D9THC) is a naturally occurring compound and is the primary active ingredient in the controlled substance marijuana. Marijuana refers to the dried flowers and leaves of Cannabis Sativa, the hemp plant. These parts of the plant contain several compounds called cannabinoids (including dronabinol), that may help patients with certain disease conditions. Dronabinol has been approved by the Food and Drug Administration (FDA) for the control of nausea and vomiting associated with chemotherapy and, more recently, for appetite stimulation of AIDS patients suffering from wasting syndrome. Synthetic dronabinol has been utilized as a pharmaceutically active ingredient, and cannabis-based medicines using botanical sources of cannibis rather than synthetic THC are also known in the art.

Currently, dronabinol is commercially available in the U.S. as a solution in a soft gelatin capsule under the tradename Marinol® from Unimed Pharmaceuticals, Inc., which is orally administered. Upon oral administration, the gelatin dissolves, releasing the drug. The dronabinol dissolved in sesame oil, is then absorbed during its passage through the gastrointestinal tract. Marinol is indicated for the treatment of: 1) anorexia associated with weight loss in patients with AIDS and 2) nausea and vomiting associated with cancer chemotherapy in patients who have failed to respond adequately to conventional antiemetic treatments. Marinol capsules are sold in 2.5 mg, 5 mg, or 10 mg dosages and formulated with the following inactive ingredients: sesame oil, gelatin, glycerin, (glycerol), methylparaben, propylparaben, and titanium dioxide. The dronabinol in the Marinol soft gelatin capsule formulation is highly unstable at room temperature, and it is recommended that the product be stored at refrigerated (2-8° C.) or cool (8-15° C.) conditions (Marinol package label, Physicians Desk Reference®, ed. 2003). Additionally, Marinol should be packaged in a well-closed container and stored in a cool environment between 8° C. and 15° C. (46° F. and 59° F.). At the present time, dronabinol and nabilone are the only approved cannabinoid drugs commercially available.

Other formulations containing dronabinol appear in the art. In 1976, Olsen et al. described a chlorofluorocarbon (CFC) propelled MDI formulation of dronabinol. Olsen, J. L., Lodge, J. W., Shapiro, B. J. and Tashkin, D. P. (1976) An inhalation aerosol of D9THC. J. Pharmacy and Pharmacol. 28:86. However, dronabinol is known to deteriorate during storage, and the stability of the dronabinol in this formulation is suspect. In addition, the ethanol content in this formulation was so high (about 23%) that an aerosol was created with droplets too large to be effectively inhaled. See, Dalby, R. N. and Byron, P. R. (1988) Comparison of output particle size distributions from pressurized aerosols formulated as solutions or suspensions. Pharm. Res. 5:36-39. The dronabinol CFC formulations were tested for use in treating asthma but were shown to be only moderately effective. See, Tashkin, D. P., Reiss, S., Shapiro, B. J., Calvarese, B., Olsen, J. L. and Lidgek, J. W. (1977) Bronchial effects of aerosolized D9THC in healthy and asthmatic subjects. Amer. Rev. of Resp. Disease. 115:57-65; Williams, S. J., Hartley, J. P. R. and Graham, J. D. P. (1976) Bronchodilator effect of D9THC administered by aerosol to asthmatic patients. Thorax. 31:720-723. Moreover, CFC propellants have since been banned so that such a formulation is now useless.

U.S. Pat. No. 6,509,005 describes an aerosol-dispensable pharmaceutical formulation comprising a hydrofluoroalkane propellant, (for example, HFA 227 or HFA 134a) and dronabinol (D9THC), which formulation is said to be stable. The propellant is present in the range of approximately 78 to 100% by weight, and more particularly the propellant is present in the range of approximately 85 to 100% by weight. An organic solvent such as ethanol can be used to assist in solubilizing the dronabinol in the propellant but it is stated that it is not required. If a solvent is used, preferably less than 20% by weight will be required, and most preferably less than 15% by weight will be required. The pharmaceutically effective concentration of dronabinol is preferably in the range of 0.05 to 10% by weight.

U.S. Pat. No. 6,747,058 and U.S. Patent Application Publication No. 2004/0162336 describe an aerosolizable formulation for delivery of delta-9-tetrahydrocannabinol in a semi-aqueous solvent, such as 35:10:55 alcohol:water:propylene glycol (v/v), which is said to produce a stable clear solution near the solubility point of the drug. These disclosures describe formulations using purified water. When the water content of the described formulations approaches 20 parts by volume, droplets form. As the formulations approach 30 parts water by volume, the dronabinol is reported to readily fall out of solution.

U.S. Pat. No. 6,383,513 describes a composition for nasal delivery comprising a cannabinoid in a biphasic delivery system, wherein the biphasic delivery system is an oil-in-water emulsion. This disclosure provides no data on long term, e.g., 2 year stability, at any conditions.

U.S. Patent Application Publication No. 2003/0229027 describes a method of preparing a pharmaceutical composition comprising a natural cannabinoid compound such as D9THC which is said to be stabilized, which comprises such a compound and a glass of a sugar, a sugar alcohol, a mixture of sugars or a mixture of sugars alcohols. The natural cannabinoid compound is dissolved in an organic solvent that is soluble in water and the sugar, sugar alcohol, mixture of sugars or mixture of sugar alcohols is dissolved in water; the dissolved cannabinoid compound and the dissolved sugar(s) are mixed; and the mixture is then dried by freeze drying, spray drying, vacuum drying, or super critical drying. The cannabinoid in this formulation is reported to withstand limited exposure to water, long enough to create a dried complex with the sugar to form a powder.

U.S. Pat. Nos. 5,508,037 and 5,389,375 describe suppository formulations prepared by admixing a therapeutically effective amount of at least one dronabinol prodrug ester derivative with a suppository base which is said to provide long term stability to the suppository formulation.

Dronabinol has been used as an antiemetic to relieve nausea and vomiting in patients receiving cancer chemotherapy. Additionally, U.S. Pat. No. 6,703,418 describes a method of treating a patient with symptomatic HIV infection to stimulate weight gain in the patient, which comprises administering to the patient a pharmaceutical composition comprising dronabinol in an amount sufficient to cause an increase in weight of the patient.

Despite all of the work outlined above and elsewhere, to date an aqueous dronabinol formulation of a cannabinoid such as dronabinol has not been achieved that is stable at room temperature over long periods of time, e.g., two years.

### OBJECTS AND SUMMARY OF THE INVENTION

All percentages of ingredients reported herein are expressed as volume to volume, unless otherwise indicated.

It is an object of the invention to provide a stabilized cannabinoid formulation, comprising an effective amount of a cannabinoid in a semi-aqueous solution buffered to a pH of from about 5 to about 10, the solution comprising water and an effective amount of an organic cosolvent to maintain the physical stability of the formulation, the formulation containing at least about 80% of the amount of cannabinoid in undegraded form after exposure of the formulation to a storage condition of (i) 40° C./60% relative humidity for 1 month; (ii) 40° C./60% relative humidity for 2 months; (iii) 40° C./60% relative humidity for 3 months; (iv) 40° C./60% relative humidity for 6 months; (v) 40° C./60% relative humidity for 8 months; (vi) room temperature (25° C.)/60% relative humidity for one year; (vii) room temperature (25° C.)/60% relative humidity for two years; and/or any combination thereof.

It is a further object of the invention to provide a stabilized cannabinoid formulation, comprising an effective amount of a cannabinoid in a semi-aqueous solution buffered to a pH of from about 5 to about 10, the solution comprising from about 20% to about 44% water and an effective amount of an organic cosolvent to maintain the physical stability of the formulation such that the formulation is not cloudy and has no visible oil droplets, the formulation containing at least about 80% of the amount of cannabinoid in undegraded form after exposure of the formulation to a storage condition of (i) 40° C./60% relative humidity for 1 month; (ii) 40° C./60% relative humidity for 2 months; (iii) 40° C./60% relative humidity for 3 months; (iv) 40° C./60% relative humidity for 6 months; (v) 40° C./60% relative humidity for 8 months; (vi) room temperature (25° C.)/60% relative humidity for one year; (vii) room temperature (25° C.)/60% relative humidity for two years; and/or any combination thereof.

It is a further object of the invention to provide a stabilized cannabinoid formulation, comprising an effective amount of a cannabinoid in a semi-aqueous solution buffered to a pH of from about 5 to about 10, the solution comprising water in an amount greater than 30% to about 44% of the formulation, and an effective amount of an organic cosolvent to maintain the physical stability of the formulation, the formulation containing at least about 80% w/w of the amount of cannabinoid in undegraded form after exposure of the formulation to a storage condition of (i) 40° C./60% relative humidity for 1 month; (ii) 40° C./60% relative humidity for 2 months; (iii) 40° C./60% relative humidity for 3 months; (iv) 40° C./60% relative humidity for 6 months; (v) 40° C./60% relative humidity for 8 months; (vi) room temperature (25° C.)/60% relative humidity for one year; (vii) room temperature (25° C.)/60% relative humidity for two years; and/or any combination thereof.

It is a further object of the invention to provide formulations comprising the following volumetric amounts: (i) from about 15 to about 50% ethanol, and (ii) a glycol that is (a) propylene glycol from about 0.1% to about 25%, (b) polyethylene glycol from about 1 to about 30%, and/or (c) a combination of (a) and (b); the formulation is suitable for administration via a nebulizer.

It is a further object of the invention to provide formulations comprising the following volumetric amounts: (i) from about 15 to about 65% ethanol and (ii) a glycol that is (a) propylene glycol from about 0.1% to about 25%, (b) polyethylene glycol from about 1 to about 25%, and/or (c) a combination of (a) and (b); the formulation being suitable for oral administration.

It is a further object of the invention to provide formulations in the form of discrete liquid droplets comprising the following volumetric amounts: (i) from about 15 to about 70% ethanol and (ii) a glycol that is (a) propylene glycol from about 0.1% to about 25%, (b) polyethylene glycol from about 1 to about 25%, and/or (c) a combination of (a) and (b); the formulation being suitable for sublingual administration.

It is a further object of the invention to provide formulations in the form of discrete liquid droplets comprising the following volumetric amounts: (i) from about 45 to about 70% ethanol and (ii) a glycol that is (a) propylene glycol from 0 to about 50%, (b) polyethylene glycol from 0 to about 2.5%, and/or (c) a combination of (a) and (b); (iii) a further solubilizing agent from 0 to about 25%; and (iv) a flavoring agent from 0 to about 1%; the formulation being suitable for sublingual administration.

It is a further object of the invention to provide a unit dose of a sublingual cannabinoid formulation comprising discrete liquid droplets of an effective amount of cannabinoid in a pharmaceutically acceptable liquid carrier suitable for sublingual spray administration; the droplets having a mean diameter of at least about 10 microns.

It is a further object of the invention to provide a unit dose or bi-dose device for sublingual administration of a drug comprising:

a reservoir containing a unit dose or a bi-dose of a liquid formulation comprising an effective amount of a cannabinoid selected from the group consisting of dronabinol, 11-OH-delta-9-THC, delta-8-THC, and 11-OH-delta-8-THC, the cannabinoid in a pharmaceutically acceptable liquid carrier comprising at least about 20% water, the carrier buffered to a pH of about 7; and the device having an actuator which when actuated delivers the unit dose of the liquid formulation in the form of liquid droplets having a mean diameter of at least about 10 microns.

It is a further object of the invention to provide a unit dose or bi-dose device for sublingual administration of a drug comprising:

a reservoir containing a unit dose or a bi-dose of a room temperature stable liquid formulation comprising an effective amount of dronabinol in a pharmaceutically acceptable liquid carrier comprising at least about 20% water, said carrier buffered to a pH of about 7; and the device having an actuator which when actuated delivers the unit dose of the liquid formulation in the form of liquid droplets having a mean diameter of at least about 10 microns.

It is a further object of the invention to provide a multi-dose device for sublingual administration of a drug comprising:

a reservoir containing a liquid formulation comprising a cannabinoid selected from the group consisting of dronabinol, 11-OH-delta-9-THC, delta-8-THC, and 11-OH-delta-8-THC, said cannabinoid in a pharmaceutically acceptable liquid carrier comprising at least about 20% water, said carrier buffered to a pH of about 7; and the device having an actuator which when actuated delivers a therapeutically effective dose of the liquid formulation in the form of liquid droplets having a mean diameter of at least about 10 microns.

It is a further object of the invention to provide a multi-dose device for sublingual administration of a drug comprising:

a reservoir containing a room temperature stable liquid formulation comprising dronabinol in a pharmaceutically acceptable liquid carrier comprising at least about 20% water, said carrier buffered to a pH of about 7; and

the device having an actuator which when actuated delivers a therapeutically effective dose of the liquid formulation in the form of liquid droplets having a mean diameter of at least about 10 microns.

It is another object of the invention to provide formulations comprising the following volumetric amounts: (i) from about 15% to about 90% ethanol, (ii) a glycol selected from the group consisting of (a) propylene glycol from about 0.1% to about 25%, (b) polyethylene glycol from about 1 to about 30%, and (c) a combination of (a) and (b), (iii) from about 0.1 to about 20% of a gelling agent, (iv) from about 0.1 to about 20% of a base and (v) from about 0.1 to about 20% of an absorption enhancer, said formulation being suitable for transdermal administration.

It is a further object of the invention to provide formulations comprising the following volumetric amounts: (i) from about 15% to about 90% ethanol, (ii) a glycol that is (a) propylene glycol from about 0.1% to about 25%, (b) polyethylene glycol from about 1 to about 30%, or (c) a combination of (a) and (b), (iii) from about 0.1 to about 20% of a gelling agent, (iv) from 0 to about 20% of a pH modifying agent and (v) from about 0 to about 20% of tonicity modifying agent, said formulation being suitable for intravenous administration.

It is another object of the invention to provide stabilized ophthalmic formulations comprising an effective amount of cannabinoid dispersed in a pharmaceutically acceptable carrier, said carrier comprising lanolin, petrolatum or combinations thereof, said formulation containing at least about 80% of the amount of cannabinoid in undegraded form after exposure of the formulation to a storage condition selected from the group consisting of (i) 40° C./60% relative humidity for 1 month; (ii) 40° C./60% relative humidity for 2 months; (iii) 40° C./60% relative humidity for 3 months; (iv) 40° C./60% relative humidity for 6 months; (v) 40° C./60% relative humidity for 8 months; (vi) room temperature (25° C.)/60% relative humidity for one year; (vii) room temperature (25° C.)/60% relative humidity for two years; and any combination thereof.

It is a further object of the invention to provide methods of treating a human patient experiencing a condition selected from the group consisting of: anorexia associated with AIDS; nausea and vomiting associated with chemotherapy; glaucoma; multiple sclerosis and pain; said method comprising the step of administering to said patient a stabilized cannabinoid formulation, comprising a cannabinoid in an effective concentration, a carrier comprising at least about 20% water, said carrier buffered to a pH range from about 6.5 to about 7.5.

It is a further object of the invention to provide methods of treatment wherein the cannabinoid formulation are suitable for administration by the delivery route selected from the group consisting of: intrapulmonary, oral, sublingual, transdermal, intravenous and ophthalmic.

It is another object of the present invention to provide a room temperature stable aqueous formulation of a cannabinoid such as dronabinol which comprises at least about 20% water and at least one cosolvent in accordance with any of the above objects.

It is a further object of the present invention to provide a room temperature stable aqueous formulation of a cannabinoid such as dronabinol which comprises an aqueous buffer in accordance with any of the above objects.

It is a further object of the invention to provide a room temperature stable formulation of a cannabinoid such as dronabinol which is thermodynamically stable in accordance with any of the above objects.

It is another object of the present invention to provide formulations that are further stabilized by the presence of a stabilizer such as a base or antioxidant in accordance with any of the above objects.

It is a further object of the invention to provide a room temperature stable formulation of a cannabinoid such as dronabinol which is readily available for absorption in the lungs of mammals, e.g., human subjects or patients.

It is a further object of certain embodiments of the present invention to provide a stable aqueous dosage form of a cannabinoid such as dronabinol for intrapulmonary administration such that the aqueous content of the dosage form does not substantially deposit the cannabinoid on the mucosal lining of the upper respiratory tract.

In accordance with any of the objects described herein, it is a further object of the invention to provide formulations suitable for intrapulmonary administration that are administered into the lung as aerosolized particles having a mean mass median aerodynamic diameter in the range of from about 0.01 to about 15 microns. Preferably the created particles have a mean mass median aerodynamic diameter in the range of from about 1 to about 10 microns, more preferably from about 2 to about 4 microns.

It is a further object of the invention to provide a room temperature stable formulation of a cannabinoid such as dronabinol which is readily available for absorption in any part of the gastrointestinal tract of mammals, e.g., human subjects or patients.

It is a further object of the invention to provide a room temperature stable formulation of a cannabinoid such as dronabinol which is readily available for absorption in the sublingual and buccal regions of mammals, e.g., human subjects or patients.

It is a further object of certain embodiments of the present invention to provide a stable aqueous dosage form of a cannabinoid such as dronabinol which can be administered sublingually in a manner which will cause substantial sublingual absorption without substantial risk of the dose passing into the lungs of the recipient.

It is a further object of the invention to provide a room temperature stable formulation of a cannabinoid such as dronabinol which is readily available for absorption through the skin of mammals, e.g., human subjects or patients.

It is a further object of the invention to provide a room temperature stable formulation of a cannabinoid such as dronabinol which is suitable for intravenous administration to mammals, e.g., human subjects or patients.

It is a further object of the invention to provide a room temperature stable formulation of a cannabinoid such as dronabinol which is readily available for absorption into the eye of mammals, e.g., human subjects or patients.

It is a further object of the present invention to provide methods and compositions for administration of a cannabinoid such as dronabinol which provides improvements over commercially available dronabinol formulations.

It is a further object of the invention to provide a stable aqueous formulation of a cannabinoid such as dronabinol that is suitable for intrapulmonary, oral, sublingual, transdermal, intravenous or ophthalmic administration for effective management of asthma, anorexia associated with weight loss in patients with AIDS, nausea and vomiting associated with cancer chemotherapy, anorexia in patients with cancer, multiple sclerosis, dystonic movement disorders, pain or glaucoma.

It is a further object of certain embodiments of the present invention to provide a method for intrapulmonary, oral, sublingual, transdermal, intravenous or ophthalmic administration of a stable aqueous formulation of a cannabinoid such as dronabinol, in a controlled amount for effective management of asthma, anorexia associated with weight loss in patients with AIDS, nausea and vomiting associated with cancer chemotherapy, anorexia in patients with cancer, multiple sclerosis, dystonic movement disorders, pain or glaucoma.

It is a further object of certain embodiments of the present invention to provide a stable aqueous liquid dosage form of a cannabinoid such as dronabinol for intrapulmonary administration.

It is a further object of certain embodiments of the present invention to provide a stable aqueous liquid dosage form of a cannabinoid such as dronabinol for oral administration.

It is a further object of certain embodiments of the present invention to provide a stable aqueous liquid dosage form of a cannabinoid such as dronabinol for sublingual or buccal administration.

It is a further object of certain embodiments of the present invention to provide a stable aqueous gel dosage form of a cannabinoid such as dronabinol which can be administered transdermally.

It is a further object of certain embodiments of the present invention to provide a stable aqueous liquid dosage form of a cannabinoid such as dronabinol which can be administered intravenously.

It is a further object of certain embodiments of the present invention to provide a stable ointment dosage form of a cannabinoid such as dronabinol which can be administered intraocularly.

In accordance with these and other objects and features, the present invention is directed in part to a room-temperature stable cannabinoid formulation comprising a therapeutically effective amount of a pharmaceutically acceptable cannabinoid in an aqueous carrier.

The invention is further directed to a cannabinoid dosage form, comprising an effective amount of a mixture of pharmaceutically acceptable cannabinoid and a pharmaceutically acceptable aqueous carrier, wherein the aqueous component also contains a buffer.

The invention is further directed in part to an aqueous formulation of a therapeutically effective amount of a dissolved cannabinoid and means for stabilizing the cannabinoid.

In further preferred embodiments of the invention where the formulation contains dronabinol as the active ingredient, the dosage form containing ingredients at a level selected from the following during its claimed shelf-life: (i) not less than 90% of the initial dronabinol content; (ii) not greater than about 2% cannabinol; (iii) not greater than about 2% delta-8-THC; and any combination of the foregoing.

In certain preferred embodiments, the present invention provides an aqueous cannabinoid formulation (e.g., dronabinol) that is stable at all conditions—refrigerated, cool and room temperature, and (2-8° C., 8-15° C. and 25° C./60% RH). In other words, in certain preferred embodiments, the stabilized aqueous cannabinoid formulations may be stored at ambient temperature and humidity, or in a refrigerator, by the patient.

In certain preferred embodiments, the cannabinoid is dronabinol formulated in the form of liquids (including suspensions and emulsions), nebulizer solution, suppositories, transdermal formulations and sublingual formulations, ophthalmic, as well as injectable formulations.

The invention is further directed in part to a method for stabilizing a dosage form containing a cannabinoid as the active pharmaceutical ingredient, comprising dissolving a therapeutically effective amount of the cannabinoid in a mixture of a pharmaceutically acceptable aqueous carrier and a pharmaceutically acceptable organic carrier, the mixture containing an effective amount of one or more stabilizing agents such as anti-oxidants.

The invention is further directed in part to a method for stabilizing a dosage form containing a cannabinoid as the active pharmaceutical ingredient, comprising dissolving a therapeutically effective amount of the cannabinoid in a pharmaceutically acceptable mixture of an aqueous and organic carrier containing an amount of one or more organic bases that is effective to stabilize the cannabinoid.

The invention is further directed in part to a method for preparing a stabilized dosage form containing a cannabinoid as the active pharmaceutical ingredient, comprising mixing a solution of a cannabinoid with an aqueous and organic carrier to obtain a flowable mixture; and further formulating the mixture in a medicament suitable for administration via the following routes: pulmonary, orally, sublingually, transdermally, intravenously or ophthalmically, wherein the formulation contains a therapeutically effective amount of said cannabinoid to provide the desired effect.

In certain embodiments, the formulations of the present invention are suitable for transmucosal administration, including, for example, buccal administration or sublingual administration.

In certain embodiments, the present invention is further directed to a method of transmucosally administering dronabinol, a pharmaceutically acceptable salt thereof, or derivative thereof, to a human in a formulation in which a substantial portion of the dronabinol, a pharmaceutically acceptable salt thereof, or derivative thereof will not be passed into the lungs of the patient. In certain preferred embodiments, the transmucosal area is the buccal area of a human.

In certain embodiments, the present invention is further directed to the use of a formulation as defined in any of the above objects for the manufacture of a medicament for use as an appetite stimulant for the management of anorexia associated with weight loss in patients with AIDS and an antiemetic for nausea and vomiting associated with cancer chemotherapy.

In accordance with certain embodiments, it is a further object of the invention to provide a method of controlling nausea and vomiting associated with a human receiving chemotherapy comprising the intrapulmonary administration of a liquid nebulizer formulation to a human patient experiencing nausea and vomiting, said liquid nebulizer formulation comprising an effective amount of a cannabinoid dispersed in a pharmaceutically acceptable carrier comprising at least about 20% water, said carrier buffered to a pH range from about 6.5 to about 7.5. Preferably, the cannabinoid is dronabinol.

In accordance with certain embodiments, it is a further object of the invention to provide a method of appetite stimulation of an AIDS patient suffering from wasting syndrome comprising the intrapulmonary administration of a liquid nebulizer formulation to a human patient experiencing a lack of appetite, said liquid nebulizer formulation comprising an effective amount of a cannabinoid dispersed in a pharmaceutically acceptable carrier comprising at least about 20% water, said carrier buffered to a pH range from about 6.5 to about 7.5. Preferably, the cannabinoid is dronabinol.

In accordance with certain embodiments, it is a further object of the invention to provide a method of controlling nausea and vomiting associated with a human receiving chemotherapy comprising the oral administration of a liquid oral formulation to a human patient experiencing nausea and vomiting, said liquid oral formulation comprising an effective amount of a cannabinoid dispersed in a pharmaceutically acceptable carrier comprising at least about 20% water, said carrier buffered to a pH range from about 6.5 to about 7.5. Preferably, the cannabinoid is dronabinol.

In accordance with certain embodiments, it is a further object of the invention to provide a method of appetite stimulation of an AIDS patient suffering from wasting syndrome comprising the oral administration of a liquid oral formulation to a human patient experiencing a lack of appetite, said liquid oral formulation comprising an effective amount of a cannabinoid dispersed in a pharmaceutically acceptable carrier comprising at least about 20% water, said carrier buffered to a pH range from about 6.5 to about 7.5. Preferably, the cannabinoid is dronabinol.

In accordance with certain embodiments, it is a further object of the invention to provide a method of controlling nausea and vomiting associated with a human receiving chemotherapy comprising the sublingual administration of a liquid sublingual formulation to a human patient experiencing nausea and vomiting, said liquid sublingual formulation comprising an effective amount of a cannabinoid dispersed in a pharmaceutically acceptable carrier comprising at least about 20% water, said carrier buffered to a pH range from about 6.5 to about 7.5. Preferably, the cannabinoid is dronabinol.

In accordance with certain embodiments, it is a further object of the invention to provide a method of appetite stimulation of an AIDS patient suffering from wasting syndrome comprising the sublingual administration of a liquid sublingual formulation to a human patient experiencing a lack of appetite, said sublingual liquid formulation comprising an effective amount of a cannabinoid dispersed in a pharmaceutically acceptable carrier comprising at least about 20% water, said carrier buffered to a pH range from about 6.5 to about 7.5. Preferably, the cannabinoid is dronabinol.

In accordance with certain embodiments, it is a further object of the invention to provide a method of controlling nausea and vomiting associated with a human receiving chemotherapy comprising the transdermal administration of a liquid, gel or semi-solid transdermal formulation to a human patient experiencing nausea and vomiting, said liquid, gel or semisolid transdermal formulation comprising an effective amount of a cannabinoid dispersed in a pharmaceutically acceptable carrier comprising at least about 20% water, said carrier buffered to a pH range from about 6.5 to about 7.5. Preferably, the cannabinoid is dronabinol and the formulation is a gel.

In accordance with certain embodiments, it is a further object of the invention to provide a method of appetite stimulation of an AIDS patient suffering from wasting syndrome comprising the transdermal administration of a liquid, gel or semi-solid transdermal formulation to a human patient experiencing a lack of appetite, said liquid, gel or semi-solid transdermal formulation comprising an effective amount of a cannabinoid dispersed in a pharmaceutically acceptable carrier comprising at least about 20% water, said carrier buffered to a pH range from about 6.5 to about 7.5. Preferably, the cannabinoid is dronabinol and the formulation is a gel.

In accordance with certain embodiments, it is a further object of the invention to provide a method of controlling nausea and vomiting associated with a human receiving chemotherapy comprising the intravenous administration of a liquid intravenous formulation to a human patient experiencing nausea and vomiting, said liquid intravenous formulation comprising an effective amount of a cannabinoid dispersed in a pharmaceutically acceptable carrier comprising at least about 20% water, said carrier buffered to a pH range from about 6.5 to about 7.5. Preferably, the cannabinoid is dronabinol.

In accordance with certain embodiments, it is a further object of the invention to provide a method of appetite stimulation of an AIDS patient suffering from wasting syndrome comprising the intravenous administration of a liquid intravenous formulation to a human patient experiencing a lack of appetite, said liquid intravenous formulation comprising an effective amount of a cannabinoid dispersed in a pharmaceutically acceptable carrier comprising at least about 20% water, said carrier buffered to a pH range from about 6.5 to about 7.5. Preferably, the cannabinoid is dronabinol.

In accordance with certain embodiments, it is a further object of the invention to provide a method of treating a human patient with glaucoma comprising the ophthalmic administration of a room temperature stable ophthalmic formulation to a human patient with glaucoma, said ophthalmic formulation comprising an effective amount of a cannabinoid dispersed in a pharmaceutically acceptable carrier selected from the group consisting of lanolin, petrolatum, and combinations thereof. Preferably, the cannabinoid is dronabinol.

In accordance with certain embodiments, it is a further object of the invention to provide a method of appetite stimulation of an AIDS patient suffering from wasting syndrome comprising the sublingual administration of a liquid sublingual formulation to a human patient experiencing a lack of appetite, said sublingual liquid formulation comprising an effective amount of a cannabinoid dispersed in a pharmaceutically acceptable carrier comprising at least about 20% water, said carrier buffered to a pH range from about 6.5 to about 7.5. Preferably, the cannabinoid is dronabinol.

In accordance with certain embodiments, it is a further object of the invention to provide a method of appetite stimulation of an AIDS patient suffering from wasting syndrome comprising the intrapulmonary administration of a liquid nebulizer formulation to a human patient experiencing a lack of appetite, said liquid nebulizer formulation comprising an effective amount of a cannabinoid dispersed in a pharmaceutically acceptable carrier comprising at least about 20% water, said carrier buffered to a pH range from about 6.5 to about 7.5. Preferably, the cannabinoid is dronabinol.

In certain preferred embodiments, the formulation contains at least about 80% w/w of the cannabinoid in undegraded form after exposure of the formulation to storage conditions selected from the group consisting of (i) 2-8° C., (ii) 25° C./60% relative humidity (RH) for 6-24 months; (iii) 30° C./60% relative humidity (RH) for 6 months; (iv) 40° C./60% relative humidity (RH) for 1-8 months; and (v) any combination thereof.

In certain embodiments, formulations and methods of the invention provide for the active pharmaceutical cannabinoid ingredient remaining within about 90 to about 110 percent of its original amount included in the dosage form for at least 1 year, and preferably at least about 2 years after manufacture.

In certain preferred embodiments, formulations of the invention are thermodynamically stable.

In certain embodiments, the cannabinoid formulations of the invention comprise effective amounts of one or more stabilizers to promote stability of the cannabinoid against unacceptable degradation. The stabilizers may comprise one or more anti-oxidants, one or more organic bases, and/or other stabilizers for cannabinoids known to those skilled in the art. In certain preferred embodiments, the stabilizer comprises povidone.

The invention is further directed in part to a method for stabilizing a dosage form containing a cannabinoid as the active pharmaceutical ingredient, comprising dissolving a therapeutically effective amount of the cannabinoid in a mixture of aqueous and organic carriers. In certain embodiments, the carrier comprises buffering agents. In certain embodiments, the carrier further comprises one or more stabilizers for the cannabinoid (e.g, anti-oxidants, organic bases, or both, as set forth more specifically herein).

In certain embodiments, the carrier further contains an effective amount of a viscosity modifier may be included to provide a pharmaceutically acceptable viscosity to the cannabinoid dispersed in the carrier. Such viscosity modifiers may be, e.g., Aerosil (silicon dioxide); cetostearyl alcohol; cetyl alcohol; stearyl alcohol; Gelucire 33/01; Gelucire 39/01; Gelucire 43/01; glyceryl behenate (Compritol 888 ATO); glyceryl palmitostearate (Precirol AT05); Softisan 100; Softisan 142; Softisan 378; Softisan 649; hydroxypropyl cellulose and mixtures thereof. In certain embodiments, the hydroxypropyl cellulose is preferred.

The invention is further directed to a dosage form wherein the cannabinoid is dronabinol and does not contain unacceptable levels of a dronabinol degradant in the dosage form selected from greater than 2% delta-8 tetrahydrocannabinol (D8THC), greater than 2% cannabinol (CBN), greater than 2% cannabidiol (CBD), and/or any combination thereof.

In certain preferred embodiments where the stabilizer comprises an organic base, the dosage form may comprise from about 0.001% w/w to about 5% organic base, preferably from about 0.001% v/v to about 0.5% organic base, by volume. In certain preferred embodiments, the organic base is selected from the group consisting of butyl hydroxyl anisole (BHA), butyl hydroxyl toluene (BHT), sodium ascorbate, and any combination of the foregoing.

The anti-oxidant included in the formulations of the invention may further be selected from e.g., propyl gallate, lecithin, Vitamin E tocopherol, sesamin, sesamol, sesamolin, alpha tocopherol, ascorbic acid, ascorbyl palmitate, fumaric acid, malic acid, and sodium metabisulphite, disodium EDTA, and combinations of any of the foregoing.

The formulations of the present invention comprise a cannabinoid concentration range of from about 0.01 to about 10 mg/ml. In certain embodiments, the formulations of the invention comprise a cannabinoid in a concentration from about 2 to about 10 mg/ml. In certain other embodiments, the formulations of the present invention comprise a cannabinoid in a formulation of about 5 mg/ml.

In certain preferred embodiments, the dosage forms of the invention comprises from about 0.05% to about 90% cannabinoid, preferably from about 0.1% to about 50% cannabinoid, more preferably about 1.5% to about 6% cannabinoid, and most preferably from about 2.5% to about 4.5% cannabinoid, by weight.

In certain embodiments wherein the formulation is a solution for pulmonary administration via nebulizer, the mixture preferably contains from about 15% to about 50% ethanol, from about 15% to about 60% buffered aqueous solution, from about 0.1 to about 25% propylene glycol and from about 1% to about 30% polyethylene glycol.

In accordance with any of the above objects, it is a further object of the invention to provide stabilized cannabinoid formulations where, the carrier is buffered to a pH of from about 5 to about 10. In certain other embodiments, the carrier is buffered to a pH of from about 6 to about 8.

In accordance with any of the above objects, the formulations of the invention are preferably buffered to a pH of from about 6.5 to about 7.5.

In accordance with any of the above objects, the formulations of the invention are preferably buffered to a pH of about 7.

In certain embodiments wherein the formulation is a solution for oral administration, the mixture preferably contains from about contains from 15% to about 65% ethanol, from about 10% to about 60% buffered aqueous solution, from about 0.1 to about 25% propylene glycol and from about 1% to about 25% polyethylene glycol. In certain preferred embodiments the oral syrup dronabinol formulations also contain a pharmaceutically acceptable sweetener such as sucrose, sorbitol and fructose in an amount from about 1% to about 10% by weight, and more preferably from about 2% to about 5% by weight.

The formulations in accordance with any of the above objects may also include sweeteners such as xylitol from about 5% to about 25%; saccharin from about 0.01% to about 5%; and saccharin sodium from about 0.01% to about 5% by weight of the formulation.

In certain embodiments wherein the formulation is a solution for sublingual administration, the mixture preferably contains from 10% to about 65% ethanol, from about 10% to about 60% buffered aqueous solution, from about 0.1 to about 25% propylene glycol and from about 1% to about 25% polyethylene glycol. In certain preferred embodiments, the sublingual dronabinol formulations also contain a flavoring agent such as mannitol in an amount from about 0.01% to about 1%.

In certain embodiments wherein the formulation is a gel for transdermal administration, the mixture preferably contains from 15% to about 90% ethanol, from about 10% to about 60% buffered aqueous solution or water, from about 0.1 to about 25% propylene glycol, from about 0.1 to about 20% of a gelling agent, from about 0.1 to about 20% of a base, from about 0.1 to about 20% of an absorption enhancer and from about 1% to about 25% polyethylene glycol. In certain other embodiments, the formulations contain propylene glycol from about 1 to about 25%.

In certain embodiments wherein the formulation is a solution for intravenous administration, the mixture preferably contains from 15% to about 90% ethanol, from about 15% to about 60% buffered aqueous solution, from about 0.1 to about 25% propylene glycol and from about 1% to about 25% polyethylene glycol.

Formulations in accordance with another aspect of the present invention are directed to a solution for ophthalmic administration that contain one or more of the following: from about 25% to about 99% lanolin, from about 25% to about 99% petrolatum, from about 1% to about 50% polyethylene glycol, from about 1% to about 50% mineral oil, and from about 1% to about 50% water or aqueous buffer solution by weight.

In certain preferred embodiments, the ophthalmic formulations contain by weight: (i) about 99% lanolin, (ii) about 25% lanolin and about 75% petrolatum, (iii) about 25% lanolin, 50% petrolatum and 25% mineral oil, (iv) about 20% lanolin, about 50% petrolatum, about 10% mineral oil and 20% water or aqueous buffer solution, or alternatively (v) from about 25% to about 99% petrolatum, from about 1% to about 50% polyethylene glycol, from about 1% to about 50% mineral oil, and from about 1% to about 50% water or aqueous buffer solution.

The invention is further directed to a dosage form which further comprises one or more additional therapeutically active agents. Non-limiting examples of such additional therapeutically active agents include a narcotic analgesic, a non-narcotic analgesic, an anti-emetic, a steroid, and mixtures of any of the foregoing.

In certain embodiments, formulations of the invention include further pharmaceutically acceptable excipients. Non-limiting examples of such pharmaceutically acceptable excipients include solubilizers for said cannabinoid, emulsifiers, absorption enhancers, surfactants, etc.

In certain preferred embodiments, the cannabinoid formulations include dronabinol as the active pharmaceutical ingredient, preferably in an amount from about 0.05 mg to about 20 mg administered orally. In other embodiments, the formulations include from about 2.5 mg to about 20 mg dronabinol administered orally.

In other preferred embodiments, for other routes of delivery such as pulmonary, sublingual, transdermal and intravenous administration, the dose of dronabinol is supplied in the amount to provide a therapeutically equivalent oral dose. In certain other embodiments that are suitable for ophthalmic administration, the dose will provide a therapeutic effective amount of a cannabinoid to treat a condition of the eye, e.g., glaucoma. In other embodiments where the cannabinoid is e.g., nabilone, 1′-OH delta-9-tetrahydrocannabinol, delta-8-tetrahydrocannabinol or 11-OH delta-8-tetrahydrocannabinol, the dose is also adjusted to account for any difference in potency to provide a dose that is therapeutically equivalent to the desire dronabinol dose. Relative activities of different cannabinoids are described in the literature. See, e.g., Razdan, Raj, K., Structure-Activity Relationships in Cannabinoids. Pharmacological Reviews, 38(2): 75-149, 1986, which is herein incorporated by reference in its entirety.

For purposes of the present invention the terms droplets and particles may be used interchangeably.

The term “pharmaceutically acceptable” is defined for purposes of the invention as meaning that a particular ingredient (e.g., pharmaceutical carrier, excipient) is not biologically or otherwise undesirable in an oral dosage form, i.e., the amount of the compound in an orally administered composition or dosage form does not cause any undesirable effects to the formulation or to the patient.

Testing for stability may be conducted, (e.g., for two year stability determination) by placing the dosage forms of the present invention under storage conditions selected from the group consisting of (i) 2-8° C., (ii) 25° C./60% relative humidity (RH) for 6-24 months; (iii) 30° C./60% relative humidity (RH) for 6 months; (iv) 40° C./60% relative humidity (RH) for 1-8 months; and (v) any combination thereof.

The phrase “does not degrade to an unacceptable extent” and the term “stable” as it applies to the cannabinoid formulations of the invention is meant for purposes of the invention to mean that the formulation contains at least about 80% w/w, and preferably at least about 90% w/w of the cannabinoid in undegraded form after exposure of the formulation to storage conditions selected from the group consisting of (i) 2-8° C., (ii) 25° C./60% relative humidity (RH) for 6-24 months; (iii) 30° C./60% relative humidity (RH) for 6 months; (iv) 40° C./60% relative humidity (RH) for 1-8 months; and (v) any combination thereof. In preferred embodiments, the phrase “does not degrade to an unacceptable extent” means that the active pharmaceutically acceptable cannabinoid ingredient (e.g., dronabinol) contained within the dosage form is maintained preferably between 90-110% of its initial (incorporated) amount during the desired (e.g., labeled) shelf-life of the dosage form (e.g., a minimum of 2 years after the date of manufacture of the dosage form).

For purposes of the invention, the term “dispersed” as it is used to describe the presence of the cannabinoid in the pharmaceutically acceptable carrier, is meant to encompass a mixture of the cannabinoid and the pharmaceutically acceptable carrier in which the cannabinoid is completely or partially dissolved therein, or the cannabinoid is partially or completely in solid particulate form therein.

For purposes of the invention, the term “unacceptable degradation” means degradation of the cannabinoid within the dosage form to an extent which will cause the dosage form to have cannabinoid in the dosage form at a level outside the acceptable ranges set forth herein, and/or which cause the formulation to include cannabinoid degradants at levels which exceed the amounts specified herein, and/or which cause the formulation to not meet its label claim for shelf life. In certain preferred embodiments, the cannabinoid formulations of the invention are deemed stable as per the FDA guidance for two-year expiration dating. In certain other preferred embodiments, the cannabinoid formulations of the invention are deemed stable as per the FDA guidance for three-year expiration dating.

### BRIEF DESCRIPTION OF THE DRAWINGS

FIG. 1 is the graphical representation of the amount (μg) of dronabinol permeated over time from the sublingual formulations of Examples 20-26 tested using Franz cells.

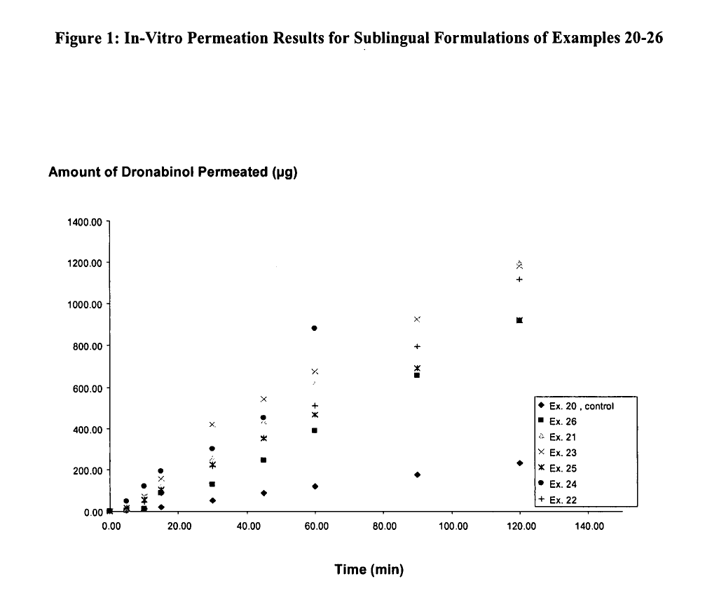

### DETAILED DESCRIPTION

Lipophilic compounds that are unstable in the presence of moisture, such as cannabinoids, have proven difficult to formulate into stable aqueous formulations due to degradation and insolubility. It has been reported that when the water content of liquid dronabinol formulations increases and the amount of organic solvent such as ethanol decreases, the drug readily falls out of solution, thus inducing instability (Dedhiya et al., 2004).

It is also believed that cannabinoid formulations designed for inhalation, such as pulmonary administration, where the organic solvent content is high are undesirable because the organic solvent rapidly evaporates upon administration, depositing the cannabinoid on the lining of the respiratory tract. This can lead to irritation of the respiratory lining.

The instability of prior art dronabinol formulations has been overcome by virtue of the present invention, which in certain embodiments (i) provides methods and formulations which provide formulations having an aqueous component, but that are nonetheless stable; (ii) significantly reduces the possibility of the dronabinol formulation being deposited on the upper respiratory lining upon inhalation; (iii) provides methods and formulations which include anti-oxidants in effective amounts to substantially prevent or slow the degradation and physical instability of the dronabinol or cannabinoid in the formulation such that, e.g., the formulation has a shelf-life of at least two years; (iv) provides methods and formulations which include organic bases (e.g., amines) in effective amounts to stabilize the dronabinol or cannabinoid in the formulation from degradation or physical instability such that, e.g., the formulation has a shelf-life of at least two years; (v) provides methods and formulations which are suitable for pulmonary, oral, sublingual, transdermal, intravenous or ophthalmic administration, or any combination of (i)-(v) above.

### Cannabinoids

Although certain sections of this specification provide specific focus on dronabinol, one skilled in the art will appreciate that the present invention is applicable to the class of pharmaceutically acceptable. For purposes of the present invention, the term “cannabinoid” includes naturally occurring and non-natural derivatives of cannabinoids which can be obtained by derivatization of natural cannabinoids and which are unstable like natural cannabinoids. In other words, the cannabinoid used in the formulations of the invention may be natural, semi-synthetic, or synthetic. The cannabinoid may be included in its free form, or in the form of a salt; an acid addition salt of an ester; an amide; an enantiomer; an isomer; a tautomer; a prodrug; a derivative of an active agent of the present invention; different isomeric forms (for example, enantiomers and diastereoisomers), both in pure form and in admixture, including racemic mixtures; enol forms. The term “cannabinoid” is also meant to encompass derivatives that are produced from another compound of similar structure by the replacement of, e.g., substitution of one atom, molecule or group by another such as 11-hydroxy-delta-8-tetrahydrocannabinol and 11-hydroxy-delta-9-tetrahydrocannabinol. The term “cannabinoid”, as used in the present invention, further includes delta-8-tetrahydrocannabinol, delta-9-tetrahydrocannabinol, cannabidiol, olivetol, cannabinol, cannabigerol, nabilone, delta-9-tetrahydro cannabinotic acid, the non-psychotropic cannabinoid 3-dimethylnepty 11 carboxylic acid homologine 8. (J. Med. Chem. 35, 3135, 1992). The term cannabinoid also includes prodrugs of cannabinoids, as well as pharmaceutically acceptable salts and complexes of cannabinoids. An example of a suitable prodrug is THC-hemisuccinate.

The term “cannabinoid” is further meant to encompass natural cannabinoids that have been purified or modified, and synthetically derived cannabinoids, for example, United States Patent Application Publication 2005/0266108, hereby incorporated by reference in its entirety, describes a method of purifying cannabinoids obtained from plant material. The term cannabinoid is also meant to include the compounds described in U.S. Pat. No. 6,713,048, including levonantradol, (−)-HU-210, Win 55212-2, Anandamide, Methandamide, CP 55940, O-1057, SR141716A, etc.). The disclosure of this patent is hereby incorporated by reference in its entirety.

In certain preferred embodiments of the present invention, the active ingredient (cannabinoid) comprises or consists essentially of Delta-9-tetrahydrocannabinol, also known as (and referred to herein as) dronabinol. Dronabinol is naturally-occurring and has been extracted from Cannabis saliva L. (marijuana). It has also been produced chemically as described in U.S. Pat. No. 3,668,224. Dronabinol is a light-yellow resinous oil that is sticky at room temperature, but hardens upon refrigeration. It turns to a flowable liquid when heated at higher temperatures. Dronabinol is insoluble in water. It has a pKa of 10.6 and an octanol-water partition coefficient: 6,000:1 at pH 7. Dronabinol is available in natural (extracted from plant) and synthetic forms. On the other hand, synthetic dronabinol may be utilized and may be synthesized using the starting materials, Olivetol and p-2,8-menthadien-2-ol (PMD).

The term “dronabinol” is further meant to encompass naturally occurring dronabinol, metabolites, synthetically derived dronabinol, and synthetically modified dronabinol starting with a molecule obtained from a natural source for example, United States Patent Application Publication 2005/0171361, hereby incorporated by reference in its entirety, describes a method of extracting delta-9-THC acid from the plant material by chromatography and then synthetically converting it to dronabinol.

The preparation of pharmaceutically acceptable cannabinoids useful in the present invention may be accomplished via any procedure known to those skilled in the art. Generally, in the isolation of THC and other cannabinoid constituents from the natural material (e.g., cannabis), the alcoholic or the petroleum ether or benzene or hexane extract of the plant is separated into neutral and acidic fractions, which are then further purified by repeated column chromatography and/or countercurrent distribution. Various adsorbents have been used in column chromatography, especially silica gel, silicic acid, silicic acid-silver nitrate, florisil, acid washed alumina, and acid washed alumina-silver nitrate. U.S. Pat. Nos. 6,365,416 and 6,730,519 describe improvements wherein Cannabis plant material is extracted with a non-polar organic solvent to provide an extract containing THC and the extract is subjected to fractional distillation under reduced pressure to provide a distillation fraction (distillate) having a high content of THC. The process further comprises subjecting the extract from the plant material to column chromatography prior to fractional distillation. A still further aspect of the process comprises subjecting the distillate from the fractional distillation to column chromatography. Additionally, the process uses high pressure liquid chromatography (HPLC) in the purification of the extract from the plant material. Another method of manufacture for obtaining cannabinoids useful in the present invention includes the method described in U.S. Pat. Nos. 6,730,519 and 6,365,416 (both to Elsohly, et al.), both hereby incorporated by reference in their entireties. Therein, a method for the isolation of delta-9-tetrahydrocannibinol (THC) from Cannabis plant material is described wherein delta-9-THC Acid and THC are separately obtained including the steps of extracting the Cannabis plant material, chelating delta-9-THC acid on alumina solid support from cannabis extracts rich in the acid washing of non-acid components of the extract with organic solvents and eluting of the delta-9-THC acid with strong polar solvents.

In certain preferred embodiments of the invention, the cannabinoid used in the formulation is esterified. Esterified forms of THC are described in U.S. Pat. No. 4,933,368 and in U.S. Pat. No. 5,389,375. Other useful polar esters are the hemi-ester of malonic acid and the alaninate ester of alanine. It has been reported, e.g., in U.S. Pat. Nos. 5,508,051 and 5,389,375, that salts of the terminal carboxylic acid group of the ester, for example, the N-methyl glutamine salt as well as the sodium and potassium salts are also useful. The descriptions of U.S. Pat. Nos. 4,933,368; 5,508,037; and 5,389,375, are incorporated herein by reference. These ester compounds are hydrolyzed in the blood stream releasing THC to provide a high degree of bioavailability of THC without regard to patient conditions and anomalies.

Oral THC is known to possess erratic absorption from the gastrointestinal tract, is subject to the first-pass effect resulting in heavy metabolism with production of high levels of 11-OH-delta-9-THC. It is reported that this 11-hydroxy metabolite is more potent agonist than delta-9-THC. The pro-drug THC hemisuccinate (THC-HS) has been formulated in a suppository base as described in U.S. Pat. Nos. 5,508,037 and 5,389,375, both of which are hereby incorporated by reference) in order to avoid this problem. Preliminary clinical investigations show promise for this formulation (Mattes, R. D.; Shaw, L. M.; Edling-Owens, J., Engleman, K.; and ElSohly, M. A.; Bypassing the first-pass effect for the therapeutic use of cannabinoids; Pharm., Biochem., Behav., 44(3):745-747, 1991; Mattes, R. D.; Engelman, K.; Shaw, L. M.; and ElSohly, M. A.; Bypassing the first-pass effect for the therapeutic use of cannabinoids, Pharmacol., Biochem., Behav., 49(1):187-195, 1994; Brenneisen, R.; Egli, A.; ElSohly, M. A.; Henn, V.; and Speiss, Y.; The effect of orally and rectally administered delta-9-tetrahydrocannabinol on spasticity: A pilot study with 2 patients; Inter. J. Clin. Pharmacol. and Therapeutics, 34(10):446-452, 1996; all of which are hereby incorporated by reference).

THC obtained by any means can be esterified by the reaction of THC with an organic acid, an organic acid halide or preferably organic acid anhydride in the presence of 4-amino-substituted pyridine alone or in admixture with an organic amine, or in any other manner known to those skilled in the art. U.S. Pat. No. 6,008,383 (Elsohly, et al.), hereby incorporated by reference, describes a process for converting dronabinol to a variety of ester analogs, which process is said to be economical and efficient. Therein, dronabinol is esterified by reaction with a carboxylic acid, an acid halide or an acid anhydride in the presence of a 4-aminopyridine either alone or in admixture with an organic amine such as a mono-, di-, or tri-alkyl amine.

In certain preferred embodiments, the cannabinoid comprises dronabinol hemisuccinate ester (THC-HS).

### Formulations

Cannabinoids in general, and dronabinol specifically, are insoluble in water. The formulations of the present invention therefore preferably include one or more pharmaceutically acceptable cosolvents for the cannabinoid. The organic cosolvent will be present in an amount effective to have the cannabinoid substantially solubilized in the organic cosolvent. Therefore, the amount of organic solvent in the formulation will vary based on the concentration of the cannabinoid. The amount of organic cosolvent will also vary based on the partition coefficient of the particular cannabinoid molecule.

### Cosolvents

In certain embodiments, the cosolvents are organic solvent such as ethanol, propanol, isopropanol, propylene glycol, polyethylene glycol, and combinations thereof that are pharmaceutically acceptable based on the intended route of administration of the desired formulation. For purposes of this invention, the term ethanol is used interchangeably with the term “absolute alcohol”. The amount of ethanol in a particular formulation will vary based on the route of delivery of the intended formulation and the solubility of the cannabinoid. The amount of ethanol in the formulations of the present invention can range from about 15 to about 90%; from about 15 to about 65%; and about 15 to about 50% by weight.

In certain preferred embodiments, polyethylene glycol is used as a portion of the cosolvent for the cannabinoid, more preferably a low molecular weight polyethylene glycol is used, most preferably polyethylene glycol 400.

In certain embodiments, the polyethylene glycol comprises from about 1% to about 40% by weight of the aqueous dronabinol formulation; from about 1% to about 30% by weight of the aqueous dronabinol formulation; from about 1% to about 25% by weight of the aqueous dronabinol formulation; more preferably from about 5% to about 30% by weight of the aqueous dronabinol formulation and most preferably from about 5% to about 25% by weight of the aqueous dronabinol formulation by weight.

In certain embodiments, the formulation contains from about 0.1% to about 30% by weight propylene glycol; from about 1% to about 30% by weight propylene glycol; from about 0.1% to about 30% by weight propylene glycol; from about 1% to about 25% by weight propylene glycol; more preferably from about 5% to about 10% of the formulation.

### Solubilizing Agents

In certain embodiments of the invention further solubilizing agents are included in the formulation. Exemplary solubilizing agents include Capryol 90; Cremophor RH40; Labrafil M 1944 CS; Labrafil M 2125 CS; Lauroglycol 90; PEG MW>4000; Plurol Oleique CC 497; poloxamer 124; poloxamer 188; Softigen 701; Softigen 767; Tagat TO; Tween 80; triacetin; triethylcitrate; tributylcitrate; acetyl triethylcitrate; acetyl tributyl citrate; ethyl oleate; ethyl caprylate; ethyl butyrate; triacetin; 2-pyrrolidone; 2-piperidone; N-methylpyrrolidone; N-ethylpyrrolidone; N-hydroxyethyl pyrrolidone; N-octylpyrrolidone; N-laurylpyrrolidone; dimethylacetamide; Miglyol, lanolin, petrolatum, mineral oil and mixtures thereof. The formulations of the present invention may comprise a solubilizing agent from about 0.1% to about 100% of the inactive ingredients; from about 5 to about 75%; or from about 25 to about 50% by weight.

Other components such as preservatives, antioxidants, surfactants, absorption enhancers, viscosity modifiers, film forming polymers, bulking agents, diluents, coloring agents, flavoring agents, pH modifiers, sweeteners or taste-masking agents may also be incorporated into any of the compositions described as part of the invention. The amount of each of these components which may be used will be optimized for each formulation, in order to obtain a stable product (dosage form) having the desired shelf-life. Generally speaking, in embodiments in which these components are included, suitable formulations may include from about 0.001% to about 20% w/w of a pharmaceutically acceptable preservative, antioxidant, surfactant, absorption enhancer, viscosity modifier, film forming polymer, bulking agent, diluent, coloring agent, flavoring agent, pH modifier, sweetener or taste-masking agent.

### Stabilizers

In certain preferred embodiments, the formulation contains amounts of one or more pharmaceutically acceptable anti-oxidants in an amount effective to stabilize the cannabinoid contained therein such that the cannabinoid does not degrade to an unacceptable extent and the formulation is deemed stable as per the ICH guidance for two-year expiration dating when placed under storage conditions selected from (i) 25° C./60% relative humidity (RH) for 12 months; (ii) 30° C./60% relative humidity (RH) for 6 months; (iii) 40° C./60% relative humidity (RH) for 6 months; and (iv) any combination thereof.

In further embodiments of the invention, an effective (stabilizing) amount of one or more pharmaceutically acceptable anti-oxidants is added to the formulation. The term “anti-oxidant” is used herein to describe any compound which is oxidized more easily than the cannabinoid compounds included in the dosage forms of the present invention. Any of the known anti-oxidants may be used, including but not limited to anti-oxidants such as butyl hydroxyl anisole (BHA), butyl hydroxyl toluene (BHT), propyl gallate, lecithin, Vitamin E tocopherol, sesamin, sesamol, sesamolin, alpha tocopherol, ascorbic acid, ascorbyl palmitate, fumaric acid, malic acid, sodium ascorbate and sodium metabisulphite, as well as chelating agents such as disodium EDTA, may also be used to stabilize the cannabinoid formulations of the present invention. Experiments described herein have shown that antioxidants like BHA, BHT and sodium ascorbate prevent degradation of dronabinol.

The preparation may also contain anti-oxidant synergists to prevent oxidative degradation. Any of the known anti-oxidant synergists may also be used in effective amounts, for example disodium edetate.

The amount of anti-oxidant which may be used will be optimized for each formulation, in order to obtain a stable product (dosage form) having the desired shelf-life. Generally speaking, in embodiments in which an anti-oxidant is included, suitable formulations may include from about 0.001% to about 20% w/w of a pharmaceutically acceptable anti-oxidant(s). For example, in certain preferred embodiments, the amount of lecithin included in the cannabinoid dosage form is in the range from about 0.1 to about 10% w/w, and in certain embodiments more preferably from about 0.3% to about 8.25% w/w. In other preferred embodiments, the amount of L-ascorbic acid-6-palmitate is from about 0.001 to about 1%, w/w, and in certain embodiments more preferably in the range from about 0.01% to about 0.1% w/w. The anti-oxidant preferably prevents the formation of degradants in the dosage form such as those mentioned above, namely delta-8 tetrahydrocannabinol (D8THC), cannabinol (CBN), or cannabidiol (CBD), to unacceptable levels (e.g., as previously specified herein).

### Bases

In further embodiments of the invention, effective amounts of one or more pharmaceutically acceptable organic or inorganic bases are added to the cannabinoid formulation in order to stabilize the cannabinoid from undesirable levels of degradation. In certain preferred embodiments, the formulation contains amounts of one or more pharmaceutically acceptable organic bases or inorganic bases in an amount effective to stabilize the cannabinoid contained therein such that the cannabinoid does not degrade to an unacceptable extent and the formulation is deemed stable as per the ICH guidance for two-year expiration dating when placed under storage conditions selected from (i) 25° C./60% relative humidity (RH) for 12 months; (ii) 30° C./60% relative humidity (RH) for 6 months; (iii) 40° C./60% relative humidity (RH) for 6 months; and (iv) any combination thereof.

Examples of suitable organic bases which may be effectively used in the cannabinoid formulations of the present invention include but are not limited to any pharmaceutically acceptable primary, secondary and tertiary organic amines which are GRAS ingredients (generally regarded as safe), such as methanolamine, ethanolamine, meglumine, other alkylamines (e.g. di-alkyl amines and tri-alkyl amines), and any combination thereof. In embodiments of the present invention where organic bases are included, suitable formulations may include from about 0.001% to about 20% w/w.

In certain preferred embodiments, the amount of organic base(s) in the formulation is from about 0.001% w/w to about 5% w/w, and more preferably from about 0.007% w/w to about 2% w/w.

In other preferred embodiments, the formulations include stabilizing amounts of both one or more anti-oxidants and one or more base.

In certain other embodiments, the formulations in accordance with the present invention are stabilized with an inorganic base e.g, NaOH, or MgOH. Generally, in embodiments in which these components are included, suitable formulations may include from about 0.001% to about 20% w/w of a pharmaceutically acceptable inorganic base.

### Buffers

In addition the formulations may additionally include physiologically acceptable components such as sodium chloride and like materials conventionally used to achieve isotonicity with typical body fluids based on the intended route of administration, e.g., the eye or intravenously. Agents which buffer the pH to maintain a physiologically compatible pH range for the intended route of administration and to enhance the solubility and stability of the active agent present, and the like may also be included in certain embodiments of the present invention.

Suitable buffers include, but are not limited to acetate, bicarbonate, citrate, phosphate, pharmaceutically acceptable salts thereof and combinations or mixtures thereof. When one or more buffers are utilized in the formulations of the invention, they may be combined, e.g., with a pharmaceutically acceptable vehicle and may be present in the final formulation, e.g., in an amount ranging from about 0.1% to about 20%, more preferably from about 0.5% to about 10%. In certain embodiments of the present invention, the amount of buffer included in the gel formulations is preferably an amount such that the pH of the gel formulation does not interfere with the body's natural buffering system causing pain. Therefore, from about 5 mM to about 200 mM concentration of a buffer may be present in the formulations. In certain preferred embodiments, from about a 20 mM to about a 100 mM concentration of a buffer is present. The concentration of buffer is such that a pH of the formulation is from about 5 to about 10; preferably from about 6 to about 8; more preferably from about 6.5 to about 7.5 and most preferably about 7.

In certain other embodiments, the formulations may be isotonic. Isotonic formulations may be provided by the addition of a tonicity agent. Suitable tonicity agents include, but are not limited to any pharmaceutically acceptable sugar, salt or any combinations or mixtures thereof, such as, but not limited to dextrose and sodium chloride. The tonicity agents may be present in an amount from about 100 mOsm/kg to about 500 mOsm/kg. In certain preferred embodiments, the tonicity agent is present in an amount from about 200 mOsm/kg to about 400 mOsm/kg and more preferably from about 280 mOsm/kg to about 320 mOsm/kg.

### Viscosity Modifiers

In further embodiments, the invention is directed to formulations that further contain viscosity modifiers including, for example, cellulose or cellulose derivatives such as ethylcellulose, hydroxyethylcellulose, hydroxypropylcellulose, carboxymethylcellulose, sodium hydroxypropylmethylcellulose, methylcellulose, methylethylcellulose, sodium carboxymethylcellulose, Aerosil, cetostearyl alcohol, Gelucires 33/01, 39/01 and 43/01, glyceryl behenate, glyceryl palmitostearate, Softisans 100, 142, 378 and 649, stearyl alcohol carbomer, xanthan gum, maltodextrin, acacia, tragacanth, povidone and polyvinyl alcohol.

### Absorption Enhancers

Absorption enhancers for use in accordance with certain embodiments of the present invention include, for example, Gelucire 44/14; Gelucire 50/13; Tagat TO; Tween 80; isopropyl myristate, polysorbates, sorbitan esters, poloxamer block copolymers, PEG-35 castor oil, PEG-40 hydrogenated castor oil, caprylocaproyl macrogol-8 glycerides, PEG-8 caprylic/capric glycerides, sodium lauryl sulfate, dioctyl sulfosuccinate, polyethylene lauryl ether, ethoxydiglycol, propylene glycol mono-di-caprylate, glycerol monocaprylate, glyceryl fatty acids (C8-C18) ethoxylated, oleic acid, linoleic acid, glyceryl caprylate/caprate, glyceryl monooleate, glyceryl monolaurate, caprylic/capric triglycerides, ethoxylated nonylphenols, PEG-(8-50) stearates, olive oil PEG-6 esters, triolein PEG-6 esters, lecithin, d-alpha tocopheryl polyethylene glycol 1000 succinate, polycarbonate, sodium glycocholate, sodium taurocholate, cyclodextrins, citric acid, sodium citrate, triacetin, combinations thereof, and the like. In certain preferred embodiments, the absorption enhancer is triacetin. In certain preferred embodiments wherein an absorption enhancer is included in the formulation, the absorption enhancer is included in an amount of from about 0.001% to about 10% by weight of the formulation, preferably in an amount of about 0.01% to about 5% by weight of the formulation.

### Bulking Agents

Bulking agents may also be used in accordance with certain embodiments of the present invention including for example, microcrystalline cellulose, mannitol, xylitol, starches and the like. In certain preferred embodiments, the bulking agent is mannitol. In certain preferred embodiments wherein bulking agent is included in the formulation, the bulking agent is included in an amount of from about 0.001% to about 10% by weight of the formulation, preferably in an amount of about 0.01% to about 5% by weight of the formulation.

### Film-Forming Polymers

In certain other embodiments of the present invention, film-forming polymers may be used for example, decreasing the fineness of the spray, the spraying angle and preferably the spreading by increasing the viscosity of the composition. As a film-forming polymer, gellan gum, xantham gum, carboxymethyl cellulose, hydroxypropyl cellulose, hydroxyethyl cellulose, hydroxypropyl methylcellulose, methylcellulose, ethylcellulose, gelucire, poloxamers, alginic acid, propyleneglycol ester, polyvinyl alcohol (PVA), polyvinylpyrrolidone (PVP), PVP/PVA copolymer, lubrajel, carboxyvinyl polymer, acrylic acid polymers and copolymers, methacrylic acid, methyl acrylate, ethyl acrylate, methyl methacrylate, ethyl methacrylate, combinations thereof and the like can be used. In certain embodiments, an increase in the viscosity of the solution using film-forming polymers or the like provides an increase in the droplet size when administered from the spray device. The chemistry of the polymer and the molecular weight of the polymer may also influence the diameter of the droplets.

### Gelling Agents

The formulations of the present invention also may contain suitable gelling or suspension agents include carbomers such as Carbopol, modified cellulose derivatives, naturally-occurring, synthetic or semi-synthetic gums such as xanthan gum, acacia and tragacanth, modified starches, co-polymers such as those formed between maleic anhydride and methyl vinyl ether, colloidal silica and methacrylate derivatives sold under the trade name Eudragit™, or a mixture thereof.

In further embodiments, additional excipients compatible with the formulations of the invention may be included corporated into the liquid drug formulation, if needed, such as known surfactants such as (e.g. Capryol 90; Cremophor RH40; Gelucire 44/14; Gelucire 50/13; Imwitor 91; Imwitor 308; Imwitor 380; Imwitor 742; Imwitor 780K; Imwitor 928; Imwitor 988; Labrafil M 1944 CS; Labrafil M 2125 CS; Lauroglycol 90; Tagat TO; Tween 80; and mixtures thereof); emulsifiers (e.g., Gelucire 44/14; Gelucire 50/13; Imwitor 91; Imwitor 308; Imwitor 380; Imwitor 742; Imwitor 780K; Imwitor 928; Imwitor 988; poloxamer 124; poloxamer 188; Tagat TO; Tween 80; lecithin; lysolecithin; phosphatidylcholine; phosphatidylethanolamine; phosphatidylglycerol; phosphatidic acid; phosphatidylserine; lysophosphatidylcholine; lysophosphatidylethanolamine; lysophosphatidylglycerol; lysophosphatidic acid; lysophosphatidylserine; PEG-phosphatidylethanolamine; PVP-phosphatidylethanolamine; sodium lauryl sulfate and mixtures thereof); and

Other additives conventionally used in pharmaceutical compositions can be included, and these additives are well known in the art. Such additives include pharmaceutically acceptable detackifiers, anti-foaming agents, chelating agents, viscomodulators, tonicifiers, flavorants, colorants odorants, opacifiers, suspending agents, binders, fillers, plasticizers, lubricants, and mixtures thereof. The amounts of such additives can be readily determined by one skilled in the art, according to the particular properties desired, keeping in mind the possibility that any such additives should preferably not negatively impact the stability of the final formulation.

Suitable coloring agents include red, black and yellow iron oxides and FD&C dyes such as FD&C Blue No. 2, FD&C Red No. 40, and the like. Suitable flavoring agents include mint, raspberry, licorice, orange, lemon, grapefruit, caramel, vanilla, cherry grape flavors, combinations thereof, and the like. Suitable pH modifiers include citric acid, tartaric acid, phosphoric acid, hydrochloric acid, maleic acid, sodium hydroxide, and the like. Suitable sweeteners include aspartame, acesulfame K, thaumatic, and the like. Suitable taste-masking agents include sodium bicarbonate, ion-exchange resins, cyclodextrin inclusion compounds, adsorbates, and the like.

It is recognized that pharmaceutical excipients may perform more than one function, and are therefore characterized as having different uses depending on the particular application. While the use of an excipient in the context of a particular formulation may determine the function of the excipient, the inclusion of any particular excipient into any one or more category as set forth above is not meant to limit the function of that excipient.

Although the ingredients of the formulations of the present invention are characterized herein as percentage based on volume, one skilled in the art will appreciate that scaled-up versions of the formulations specifically described herein may be characterized instead on a weight percentage basis. Where the density of a particular component is 1 g/ml the amount of the component based on volume and weight will be the same. Where the density deviates from 1 g/ml, the amounts based on weight or volume will differ accordingly.

### Additional Drugs

Cannabinoids such as dronabinol may be used alone or in combination with other medications. Those skilled in the art will readily recognize that, for example, in the case of AIDS wasting syndrome, the patient will likely also be taking drugs that combat the AIDS virus. Similarly, those skilled in the art will readily recognize that patients receiving chemotherapy for cancer may also receive other antiemetics, and cancer patients seeking to relieve pain are likely to receive opioids as well as nonsteroidal anti-inflammatory agents. The formulations and methods of the invention may further include one or more additional therapeutically active agents, such as, for example, non-narcotic analgesics such as acetaminophen or aspirin, opioid or opiate analgesics, non-steroidal anti-inflammatory drugs (NSAIDs, for example, non-selective cyclooxygenase inhibitors and COX-2 inhibitors), anti-emetics (for example, ondansetron) and steroids (for example megestrol acetate, oxandrolone, oxymetholone). In certain embodiments of the invention, a second therapeutically active drug including but not limited to the above-mentioned drugs, is incorporated into the oral cannabinoid dosage form. In yet other embodiments, the second therapeutically active drug is separately administered to the patient in conjunction with the oral cannabinoid dosage form. The sequence in which the therapeutic agents are administered is not narrowly critical. “Combination therapy” embraces the administration of the therapeutic agents as described above in further combination with other biologically active ingredients, such as, but not limited to, a pain reliever, such as a steroidal or nonsteroidal anti-inflammatory drug, or an agent for improving stomach motility, for example, and with non-drug therapies, such as, but not limited to, surgery.

The therapeutic compounds that make up the combination therapy may also be administered sequentially, with either therapeutic compound being administered by a regimen calling for two-step administration. Thus, a regimen may call for sequential administration of the therapeutic compounds with spaced-apart administration of the separate, active agents. The time period between the multiple administration steps may range from, for example, a few minutes to several hours to days, depending upon the properties of each therapeutic compound such as potency, solubility, bioavailability, plasma half-life and kinetic profile of the therapeutic compound, as well as depending upon the effect of food ingestion and the age and condition of the subject. Circadian variation of the target molecule concentration may also determine the optimal dose interval.

The therapeutic compounds of the combined therapy whether administered simultaneously, substantially simultaneously, or sequentially, may involve a regimen calling for administration of one therapeutic compound by oral route and another therapeutic compound by an oral route, a percutaneous route, an intravenous route, an intramuscular route, or by direct absorption through mucous membrane tissues, for example. Whether the therapeutic compounds of the combined therapy are administered orally, rectally, topically, buccally, sublingually, or parenterally (for example, subcutaneous, intramuscular, intravenous and intradermal injections, or infusion techniques), separately or together, each such therapeutic compound will be contained in a suitable pharmaceutical formulation of pharmaceutically-acceptable excipients, diluents or other formulations components.

### Nebulizer Formulations

Pulmonary administration offers a route of delivery that is suitable for administration of drugs wherein the drug properties make it difficult for oral administration, or where the physical state of the specific patient does not make oral administration desirable (e.g., vomiting, compromised gastrointestinal tract).

In certain embodiments, the formulations of the present invention are designed for pulmonary delivery via a nebulizer. Nebulizers are broadly known to those of skill in the art and the invention is not limited to any specific type of nebulizer. Examples of suitable nebulizers and/or delivery devices and their method of use that are suitable for pulmonary administration of the formulations disclosed herein are described in: U.S. Pat. Nos. 7,036,500; 7,029,656; 7,013,894; 6,994,083; 6,962,151; 6,929,003; 6,854,662; 6,748,945; 6,732,731; 6,729,327; 6,598,602; 5,853,002; 5,549,102; 5,435,282; 5,036,840; 7,077,126; 7,059,320; 6,983,747; 6,679,251; 6,606,990; 6,514,177; 513,727; 6,513,519; 6,464,388; 6,176,237; 6,085,741; 6,000,394; 5,957,389; 5,740,966; 5,596,982; 5,461,695; 5,458,136; 5,312,046; 5,309,900; 5,280,784; and U.S. Patent Publication Nos.: 20060102172; 20060065267; 20060054166; 20060048772; 20060011196; 20050224076; 20050056274; 20050039741; 20040250816; 20030037788; 20030037785; 20020005196 and 20010054421 and the like which are suitable for intrapulmonary administration. The disclosures of which are incorporated by reference in their entireties.

In certain preferred embodiments, the nebulizer used in accordance with the present invention is the Pari LC STAR, LC Sprint or LC Plus. In more preferred embodiments, the nebulizer is the Pari LC Sprint Star.

One of skill will readily understand that the stable aqueous cannabinoid formulations of the present invention can be incorporated into any suitable nebulizer, and provide delivery of the active ingredient to the lungs.

In certain embodiments, formulations suitable for intrapulmonary administration are administered into the lung as aerosolized particles having a mean mass median aerodynamic diameter in the range of from about 0.01 to about 15 microns. Preferably the created particles have a mean mass median aerodynamic diameter in the range of from about 1 to about 10 microns, more preferably from about 2 to about 4 microns.

### Oral Syrup Formulations

In further embodiments, the present invention is formulated into a stable aqueous cannabinoid formulation by first preparing a stable aqueous nebulizer formulation, and then further adding a sweetening agent, taste-masking agent, flavoring agent, coloring agent, viscosity modifying agent or combinations thereof.

In accordance with oral syrup formulations of the current invention where the cannabinoid is dronabinol, the dronabinol concentration is from about 0.05 mg/ml to about 100 mg/ml; preferably from about 0.5 mg/ml to about 10 mg/ml; and more preferably about 1 mg/ml. The dose of dronabinol provided by the oral syrup formulations is preferably from about 2.5 mg to about 50 mg dronabinol.

In certain embodiments, the present invention further contains a viscosity modifying agent, e.g. hydroxypropylcellulose or polyvinylpyrrolidone (povidone or PVP).

In certain embodiments the invention is directed to stable aqueous cannabinoid formulations for oral administration that contains sucrose, fructose, sorbitol, xylitol, saccharin, saccharin sodium or combinations thereof as a sweetening agent.

One of skill will readily appreciate that the stable aqueous cannabinoid oral liquid formulations of the present invention can be incorporated into any pharmaceutically acceptable single-dose or multi-dose container made from any pharmaceutically acceptable material, (e.g., glass or plastic) to allow for oral dosing of the formulation.

### Sublingual Formulations

The oral cavity offers a simple, painless method of cannabinoid administration. Within the oral cavity, there are three generally recognized routes of administration of an active agent, namely local, buccal and sublingual.

Local delivery is mainly limited to applications regarding disruptions occurring within the oral cavity itself, such as a canker sore.

The buccal mucosa area encompasses the mucosal membranes of the inner lining of the cheeks. The buccal mucosa is however, less permeable than the sublingual area. One of the major disadvantages associated with buccal mucosa delivery of an active agent has been the relatively low passage of active agents across the mucosal epithelium, thereby resulting in low agent bioavailability, which translates into a substantial loss of usable active agent within each dosage.

Sublingual delivery is achieved through the mucosal membranes lining the floor of the mouth. Because of the high permeability and the rich blood supply, transport via the sublingual route results in rapid absorption. Sublingual delivery is also beneficial in providing a delivery route appropriate for highly permeable drugs with short delivery period requirements and an infrequent dosing regimen.

The sublingual formulations of the present invention are useful management of anorexia associated with weight loss in patients with AIDS and nausea and vomiting associated with cancer chemotherapy.

Sublingual administration of dronabinol, a pharmaceutically acceptable salt thereof, or derivative thereof, in accordance with the present invention may be particularly beneficial in the patient with cancer who is unable to tolerate oral administration because of nausea and vomiting, dysphagia as a result of disease, or parenteral administration because of decreased venous access, emaciation, or coagulation defects. Sublingual administration of dronabinol in accordance with the present invention preferably has potential advantages of even greater ease of use and rapid onset of appetite stimulant or antiemetic action. Furthermore, because sublingual venous drainage is systemic rather than portal, hepatic first-pass elimination may be avoided. The present invention preferably provides therapeutic formulations and methods for solutions of dronabinol, a pharmaceutically acceptable salt thereof, or derivative thereof to be delivered by sublingual spray pumps.

In certain preferred embodiments, the sublingual administration of dronabinol, a pharmaceutically acceptable salt thereof, or derivative thereof, is advantageous over other forms of administration in that it does not require injection using a syringe and needle, and avoids the need for formulating unit dose oral formulations. Preferably the sublingual administration of dronabinol, a pharmaceutically acceptable salt thereof, or derivative thereof, in accordance with the present invention is suitable for self administration.

In certain embodiments, the formulations of the present invention are advantageous in that propellants such as hydrofluorocarbon propellants such as volatile chlorofluocarbons (e.g. propellant 12), volatile hydrofluoroalkanes (e.g. 1,1,1,2-tetrafluoroethane and 1,1,1,2,3,3,3-heptafluoro-n-propane) and volatile alkanes (e.g. propane, butane) are not required to deliver the dronabinol, a pharmaceutically acceptable salt thereof, or derivative thereof, sublingually to the patient.

Preferably the formulations of the present invention are delivered as liquid droplets having a mean diameter of at least about 10 microns, preferably at least about 20 microns, more preferably a mean diameter of from about 20 to about 200 microns. Most preferably the formulations are delivered as liquid droplets have a size distribution of from about 5 microns to about 500 microns, preferably from about 10 microns to about 200 microns, preferably from about 20 microns to about 100 microns, more preferably from about 30 microns to about 70 microns.

Preferably the delivery of the formulation of the present invention to the sublingual mucosa via spray results in a rapid absorption of the dronabinol, a pharmaceutically acceptable salt thereof, or derivative thereof.

In certain embodiments, the formulations of the present invention are designed for sublingual administration.

In certain embodiments the present invention is directed to a sublingual dronabinol formulation comprising discrete liquid droplets comprising an effective amount of dronabinol, a pharmaceutically acceptable salt thereof, or derivative thereof, said droplets having a mean diameter of at least about 10 microns, preferably at least about 20 microns, more preferably a mean diameter of from about 20 to about 200 microns.

In certain embodiments, the present invention is directed to a sublingual dronabinol formulation comprising discrete liquid droplets of dronabinol, a pharmaceutically acceptable salt thereof, or derivative thereof; in a pharmaceutically acceptable liquid carrier; said droplets having a size distribution of from about 5 microns to about 500 microns, preferably from about 10 microns to about 200 microns, preferably from about 20 microns to about 100 microns, more preferably from about 30 microns to about 70 microns.

In certain preferred embodiments of sublingual formulations, none of the particles have a diameter which would allow the dronabinol, pharmaceutically acceptable salt thereof, or derivative thereof to be delivered to the lung upon sublingual administration.

In certain embodiments, the present invention is directed to a unit dose of a sublingual dronabinol formulation, said unit dose comprising discrete liquid droplets of dronabinol, a pharmaceutically acceptable salt thereof, or derivative thereof; and a pharmaceutically acceptable liquid carrier; said droplets having a mean diameter of at least about 10 microns, preferably at least about 20 microns, more preferably a mean diameter of from about 20 to about 200 microns.

In certain embodiments, the present invention is directed to a unit dose of a sublingual dronabinol formulation, said unit dose comprising discrete liquid droplets of dronabinol, a pharmaceutically acceptable salt thereof, or derivative thereof, and a pharmaceutically acceptable liquid carrier; said droplets having a size distribution of from about 5 microns to about 500 microns, preferably from about 10 microns to about 200 microns, preferably from about 20 microns to about 100 microns, more preferably from about 30 microns to about 70 microns.

In certain embodiments, the present invention is directed to a method of effective management of anorexia associated with weight loss in patients with AIDS comprising sublingually administering a liquid spray formulation in the form of discrete liquid droplets having a mean diameter of at least about 10 microns, preferably at least about 20 microns, more preferably a mean diameter of from about 20 to about 200 microns, to a human patient experiencing anorexia, said liquid spray formulation comprising an effective amount of dronabinol, a pharmaceutically acceptable salt thereof, or derivative thereof, dispersed in a pharmaceutically acceptable liquid carrier.

In certain embodiments, the present invention is directed to a method of effective management of nausea and vomiting associated with cancer chemotherapy comprising sublingually administering a liquid spray formulation in the form of discrete liquid droplets having a mean diameter of at least about 10 microns, preferably at least about 20 microns, more preferably a mean diameter of from about 20 to about 200 microns, to a human patient experiencing nausea and vomiting associated with cancer chemotherapy, said liquid spray formulation comprising an effective amount of dronabinol, a pharmaceutically acceptable salt thereof, or derivative thereof, dispersed in a pharmaceutically acceptable liquid carrier.

In certain embodiments, the present invention is directed to a method of effective management of anorexia associated with weight loss in patients with AIDS comprising sublingually administering a liquid spray formulation in the form of discrete liquid droplets having a size distribution of from about 5 microns to about 500 microns, preferably from about 10 microns to about 200 microns, preferably from about 20 microns to about 100 microns, more preferably from about 30 microns to about 70 microns to a human patient experiencing anorexia; said liquid spray formulation comprising an effective amount of dronabinol, a pharmaceutically acceptable salt thereof, or derivative thereof, dispersed in a pharmaceutically acceptable liquid carrier.

In certain embodiments, the present invention is directed to a method of effective management of nausea and vomiting associated with cancer chemotherapy comprising sublingually administering a liquid spray formulation in the form of discrete liquid droplets having a size distribution of from about 5 microns to about 500 microns, preferably from about 10 microns to about 200 microns, preferably from about 20 microns to about 100 microns, more preferably from about 30 microns to about 70 microns to a human patient experiencing nausea and vomiting associated with cancer chemotherapy; said liquid spray formulation comprising an effective amount of dronabinol, a pharmaceutically acceptable salt thereof, or derivative thereof, dispersed in a pharmaceutically acceptable liquid carrier.

In certain embodiments, the present invention is directed to a device which includes a reservoir containing a unit dose of a liquid formulation comprising an effective amount of dronabinol, a pharmaceutically acceptable salt thereof, or derivative thereof in a pharmaceutically acceptable liquid carrier; the device having an actuator which when actuated delivers the unit dose of the liquid formulation in the form of liquid droplets having a mean diameter of at least about 10 microns, preferably at least about 20 microns, more preferably a mean diameter of from about 20 to about 200 microns. Preferably, the device delivers a therapeutically effective dose of the liquid formulation in the form of liquid droplets having a size distribution of from about 5 microns to about 500 microns, preferably from about 10 microns to about 200 microns, preferably from about 20 microns to about 100 microns, more preferably from about 30 microns to about 70 microns.

In certain embodiments, the present invention is directed to a multi-dose device which includes a reservoir containing a liquid formulation comprising dronabinol, a pharmaceutically acceptable salt thereof, or derivative thereof in a pharmaceutically acceptable liquid carrier; the device having an actuator which when actuated delivers a therapeutically effective dose of the liquid formulation in the form of liquid droplets having a mean diameter of at least about 10 microns, preferably at least about 20 microns, more preferably a mean diameter of from about 20 to about 200 microns. Preferably, the device delivers a therapeutically effective dose of the liquid formulation in the form of liquid droplets having a size distribution of from about 5 microns to about 500 microns, preferably from about 10 microns to about 200 microns, preferably from about 20 microns to about 100 microns, more preferably from about 30 microns to about 70 microns.

In certain embodiments, the present invention is directed to a method of effective management of anorexia associated with weight loss in patients with AIDS comprising utilizing a spray device which includes a reservoir including a liquid formulation comprising dronabinol, a pharmaceutically acceptable salt thereof, or derivative thereof in a pharmaceutically acceptable liquid carrier; and an actuator which upon actuation delivers a therapeutically effective amount of liquid droplets to be sprayed from the device having a mean diameter of at least about 10 microns, preferably at least about 20 microns, more preferably a mean diameter of from about 20 to about 200 microns.

In certain embodiments, the present invention is directed to a method of effective management of anorexia associated with weight loss in patients with AIDS comprising utilizing a spray device which includes a reservoir including a liquid formulation comprising dronabinol, a pharmaceutically acceptable salt thereof, or derivative thereof in a pharmaceutically acceptable liquid carrier; and an actuator which upon actuation delivers a therapeutically effective amount of liquid droplets to be sprayed from the device having a mean diameter of at least about 10 microns, preferably at least about 20 microns, more preferably a mean diameter of from about 20 to about 200 microns.

In certain embodiments, the present invention is directed to a method of treating nausea and vomiting associated with cancer chemotherapy comprising utilizing a spray device which includes a reservoir including a liquid formulation comprising dronabinol, a pharmaceutically acceptable salt thereof, or derivative thereof; and a pharmaceutically acceptable liquid carrier; and an actuator which upon actuation delivers a therapeutically effective amount of liquid droplets having a size distribution of from about 5 microns to about 500 microns, preferably from about 10 microns to about 200 microns, preferably from about 20 microns to about 100 microns, more preferably from about 30 microns to about 70 microns.

In certain embodiments, the formulations of the present invention are suitable for transmucosal administration, including, for example, buccal administration.

In certain embodiments, the present invention is further directed to a method of transmucosally administering dronabinol, a pharmaceutically acceptable salt thereof, or derivative thereof, to a human in a formulation in which a substantial portion of the dronabinol, a pharmaceutically acceptable salt thereof, or derivative thereof will not be passed into the lungs of the patient. In certain preferred embodiments, the transmucosal area is the buccal area of a human.

In certain embodiments, the present invention is further directed to the use of a formulation as defined above for the manufacture of a medicament for use as an appetite stimulant for the management of anorexia associated with weight loss in patients with AIDS and an antiemetic for nausea and vomiting associated with cancer chemotherapy.

In certain embodiments, the formulations according to the invention are preferably packaged as a bulk solution containing multiple doses in a pump spray system comprising a sealed container fitted with a metering pump.

In certain alternate embodiments the formulations according to the invention are preferably package as a single unit dose solution in a single unit dose pump spray system comprising a sealed container fitted with a pump.

Typically a patient is treated by administration sublingually of 1 to 2 actuations, from the spray pump. Another advantage of sublingual spray delivery is the ability to easily titrate patients by 1 or 2 doses as required by a single actuation. This is typically not the case with other forms of drug delivery (patches, lozenges, tablets, suppositories).

Pump action sprays are characterized in requiring the application of external pressure for actuation, for example, external manual, mechanical or electrically initiated pressure. This is in contrast to pressurized systems, e.g., propellant-driven aerosol sprays, where actuation is typically achieved by controlled release of pressure e.g., by controlled opening of a valve.

In certain embodiments the pump sprays are preferred as the use of a pump spray with the formulation of the present invention allows for the administration of droplets or particles having a mean diameter of at least about 10 microns, preferably at least about 20 microns, more preferably a mean diameter of from about 20 to about 200 microns, and/or preferably having a size distribution of from about 5 microns to about 500 microns, preferably from about 10 microns to about 200 microns, preferably from about 20 microns to about 100 microns, more preferably from about 30 microns to about 70 microns. This is in contrast to a pressurized system which may result in particles less than 5 microns. Liquid droplets or particles having a diameter of less than about 5 microns have the potential to enter into the lungs of a human upon administration. Such entry into the lungs could lead to an increase in patient to patient variability in absorption of the dronabinol. Further, absorption of dronabinol in the lungs could lead to an increased absorption and increased side effects, including respiratory depression which may be fatal.

In certain preferred embodiments, the droplet size of the delivered formulations further provides for an increase in surface area by being sprayed sublingually as opposed to being placed under the tongue with e.g., a dropper.

In certain preferred embodiments, the delivery device is a device such as those described in U.S. Pat. Nos. 6,866,566; 6,877,672; 6,772,915; 6,725,857; 6,705,493; 6,679,248; 6,578,741; 6,527,144; 6,484,715; 6,478,196; 6,461,322; 6,446,839; 6,427,878; 6,367.473; 6,364,166; 6,321,942; 6,234,366; 6,227,413; 6,059,151; 6,059,150; 6,055,979; 5,944,222; 5,901,883; 5,813,570; 4,565,302; 4,532,967; 6,964,381; 6,860,411; 6,824,020; 6,817,490; 6,585,172; 6,443,370; 6,427,680; 6,425,499; 6,401,987; 6,398,074; 6,264,065; 5,950,877; 5,328,099; 5,301,846; and the like which are described in certain embodiments as being suitable for nasal administration.

Other devices suitable for use in accordance with the formulations of the present invention are described in U.S. Pat. Nos. 6,808,085; 6,736,293; 6,732,955; 6,708,846; 6,626,379; 6,626,330; 6,626,328; 6,454,185; 6,427,876; 6,427,684; 6,419,167; 6,405,903; 6,352,181; 6,308,867; 6,257,461; 6,257,454; 6,250,509; 6,227,415; 6,209,760; 6,179,164; 6,109,547; 6,062,430; 6,026,992; 5,992,704; 5,992,703; 5,988,449; 5,967,369; 5,964,417; 5,950,879; 5,938,125; 5,927,559; 5,921,444; 5,893,484; 5,875,938; 5,862,962; 5,860,567; 5,816,504; 5,813,570; 5,803,311; 5,791,518, 5,692,650; 5,655,689; 5,584,417; 5,520,337; 5,519,980; 5,482,193; 5,469,989; 5,443,185; 5,439,177; 5,437,398; 5,427,280; 5,395,032; 5,375,745; 5,368,201; 5,366,122; 5,366,122; 5,335,823; 5,326,000; 5,323,936; 5,316,198; 5,301,841; 5,295,628; 5,289,946; 5,277,334; 5,257,726; 5,228,586; 5,209,375; 5,203,840; 5,147,087; 5,115,980; 5,110,052; 5,011,046; 4,958,752; 4,946,069; 4,944,430; 4,934,568; 4,921,142; 4,871,092; 4,830,284; 4,826,048; 4,823,991; 4,821,923; 4,817,829; 4,776,498; 4,762,475; 4,728,008; 4,726,747; 4,694,977; 4,694,976; 4,566,611; 6,851,583; 6,824,021; 6,779,690; 6,776,312; 6,971,559; 6,948,640; 6,945,473; 6,938,802; 6,933,850; 6,929,156; 6,918,514; 6,913,205; 6,866,168; 6,832,072; 6,830,163; 6,817,490; 6,817,489; 6,811,060; 6,811,057; 6,805,301; 6,805,263; 6,789,750; 6,789,706; 6,786,369; 6,783,035; 6,772,913; 6,769,579; 6,758,371; 6,752,298; 6,742,677; 6,705,062; 6,698,627; 6,698,623; 6,663,019; 6,659,314; 6,659,307; 6,655,550; 6,655,549; 6,651,846; 6,601,735; 6,595,395; 6,592,010; 6,588,629; 6,581,852; 6,571,991; 6,554,160; 6,536,635; 6,527,149; 6,527,148; 6,488,185; 6,471,097; 6,460,781; 6,460,740; 6,460,738; 6,446,841; 6,422,429; 6,409,049; 6,398,079; 6,360,919; 6,349,856; 6,345,737; 6,343,722; 6,662,561; 6,315,169; 6,273,303; 6,273,300; 6,261,274; 6,257,457; 6,234,363; 6,234,168; 6,221,054; 6,209,759; 6,189,741; 6,186,371; 6,155,496; 6,119,897; 6,105,826; 6,021,930; 6,012,615; 5,988,496; 5,950,871; 5,931,386; 5,850,948; 5,803,318; 5,799,810; 5,769,325; RE35,683; 5,692,492; 5,568,884; 5,566,865; 5,511,698; 5,482,188; 5,476,198; 5,366,115; 5,337,923; 5,249,713; 5,237,797; 5,234,135; 5,226,563; 5,190,192; 5,176,296; 5,127,548; 4,966,313; 4,91,840; 4,245,967; 4,030,667; and the like.

All of the patents recited herein are hereby incorporated by reference in their entireties. Although the delivery devices disclosed in the patents described above may be suitable for nasal or inhalation administration, in accordance with certain embodiments of the present invention the delivery devices are specifically adapted to be suitable for sublingual administration of a liquid formulation.

Preferably the device in accordance with the present invention is adapted to sublingually deliver the sublingual formulation in a controlled manner preferably such that only droplets having a mean diameter of at least about 10 microns, preferably at least about 20 microns, more preferably a mean diameter of from about 20 to about 200 microns are delivered to the patient. More preferably only droplets having a size distribution in the range of from about 5 microns to about 500 microns, preferably from about 10 microns to about 200 microns, preferably from about 20 microns to about 100 microns, more preferably from about 30 microns to about 70 microns.

Preferably the dispenser is constructed in such a way that it can be carried and simultaneously reliably operated with the fingers, or with three fingers of one hand, and can be used, for example, in the manner of a sublingual spray. The dispenser can be constructed as a disposable dispenser which, following the emptying of the medium chamber, does not have to be refilled and can therefore be constructed as a simple standard component, which receives the pump, the formulation, the channels and optionally, valves or closures within an outer casing, which in side view can be roughly T-shaped or Y-shaped.

If the dispenser is to be emptied in a single pump stroke in successive portions or in one complete pump stroke, and is not to be refilled, then the dispenser can be substantially tightly closed with respect to the outside in the starting position

In certain preferred embodiments, the delivery device (e.g., such as a spray pump device) includes a lock-out mechanism. Preferably the lock-out mechanism allows for administration of only one unit dose, and preferably prevents abuse of the dronabinol, a pharmaceutically acceptable salt thereof, or derivative thereof, by only allowing for the administration of one dose and locking out of further administration for a certain and/or predetermined period of time. In certain embodiments, after one or more actuating cycles the actuator can be automatically transferred into the locking position, so that for performing a following actuating cycle randomly or deliberately a release must take place. Locking can take place in the starting position, actuating position and/or an intermediate position and can act both against actuation and against return or against one of these movements alone and several locking positions with the same or different locking action are possible.

In certain embodiments, the device may be premetered or alternatively, the device may be device-metered. Premetered devices preferably contain previously measured doses or a dose fraction in some type of units (e.g., single unit dose amount of solution, single or multiple blisters or other cavities) that may be included in the device during manufacture or by the patient before use. Typical device-metered units have a reservoir containing formulation sufficient for multiple doses that are delivered as metered sprays by the device itself when activated by the patient.

Important factors to consider with manufacture of the device are the reproducibility of the dose, the spray plume, and the particle/droplet size distribution, which can affect the delivery of the dronabinol, a pharmaceutically acceptable salt thereof, or derivative thereof, under the tongue. Maintaining the reproducibility of these parameters through the expiration dating period and ensuring the functionality of the device (e.g., spray mechanism, electronic features, sensors) through its lifetime under patient-use conditions is important as any alteration in these parameters could lead to variability in dosing and absorption, which could lead to potential side effects.

The administered dose of spray drug formulation may be dependent on the design, reproducibility, and performance characteristics of the container closure system. A suitable device which provides the desired droplet/particle size distribution is an important factor for the correct performance of the dronabinol product. Actuation parameters (e.g., force, speed, hold and return times) should also be considered with respect to the device. Moreover, the device should be compatible with formulation components. Further, the device should be designed to prevent partial metering of the dronabinol, a pharmaceutically acceptable salt thereof, or derivative thereof, formulation when used according to the patient instructions for use.

A typical device includes a base unit, a discharge actuator, an orifice for the formulation to be release from the device, and a medium reservoir. Preferably a reservoir is provided which as a dispensing chamber is filled already on production of the device. The medium reservoir preferably defines a measured content of dronabinol, a pharmaceutically acceptable salt thereof, or derivative thereof, to be discharged upon a single activation.

In accordance with certain embodiments of the invention, a reservoir, or a space thereof receiving the medium is preferably an elongated shape preferably having a wall thickness which is constant over the circumference and length of the reservoir body. The reservoir body may be formed simply by a section of a cylindrical hollow of plastic, steel, such as stainless steel, transparent material, such as glass, or the like so that its production is very simple.

Preferably an actuator body is provided on a unit of the device, which is movable relative to the orifice for activating discharge. This body, in the course of the actuating movement, opens a closure of a chamber, e.g. by puncturing. The space within this chamber may directly adjoin the medium in the reservoir, accommodate the opening body or the reservoir at least in part and configured as a pressure space which prior to being opened is at an elevated pressure. The opening body may be formed directly by the reservoir.

Preferably during a part of the actuating travel following the starting position an elevated pressure is built up. In a subsequent portion of the actuating movement continuing in the same direction, the medium is relieved of the pressure at one of the sides and communicated to the medium orifice on this side. As such, due to the pressure acting on the side, the medium is pushed from the reservoir and through the orifice.

Typically as the liquid formulation leaves the orifice, the liquid droplets follow a trajectory which is influenced by the orifice shape of the device. In certain embodiments, the droplet size, spray geometry, and the spray pattern are dependent on the design of the pump and/or the properties of the formulation. In certain embodiments, the orientation of the actuator, pump design, and the properties of the formulation will influence the spray symmetry and the shape.

In certain embodiments, the device of the present invention further includes a stopper. Preferably the stopper comprises a material which precludes or substantially precludes the absorption of the dronabinol, pharmaceutically acceptable salt thereof, or derivative thereof. A suitable stopper for use in accordance with the device of the present invention is, for example, a stopper marketed by West Pharmaceutical Services, Inc. In certain preferred embodiments, the stopper has the following composition and characteristic: 1) elastomer: bromobutyl and/or chlorobutyl; 2) reinforcement: inert material; and 3) curing system: unconventional.

In certain embodiments, the device further includes a gasket. Preferably the gasket comprises a material which precludes or substantially precludes the absorption of the dronabinol, pharmaceutically acceptable salt thereof, or derivative thereof. A suitable gasket for use in accordance with the device of the present invention is, for example, a stopper marketed by West Pharmaceutical Services, Inc. In certain preferred embodiments, the gasket has the following composition and characteristic: 1) elastomer: bromobutyl and/or chlorobutyl; 2) reinforcement: inert material; and 3) curing system: unconventional.

Droplet size distribution can be determined using laser diffraction methodology, such as for example, Malvern Spraytec® with RT Sizer software. A Malvern Mastersizer S, by Malvern Instruments Limited (U.K.), device may also be used to determine size distribution. A Malvern Mastersize S is a modular particle size analyzer offering measurement versatility. It can measure spray droplet size as well as wet and dry samples. Particles from sub-micron to a few millimeters may be measured with the Malvern Mastersizer S. Further, automated actuation stations for comparative in vitro bioequivalence tests to decrease the variability associated with manual actuation may also be used when determining the droplet size distribution.

### Transdermal Gel Preparations

Transdermal administration provides a route of administration where other routes such as oral and pulmonary are not suitable. These formulations are preferably prepared by adding and mixing one or more gelling agents, a suitable base and one or more absorption enhancers to the above-mentioned nebulizer formulations. The gel is transferred into suitable container made from a pharmaceutically acceptable material, e.g., plastic or glass for convenient administration. The dosage ranges will vary with the choice of cannabinoid, however in certain embodiments where the cannabinoid is dronabinol, the dose will be adjusted to provide a dose that is therapeutically equivalent to the oral dose of Marinol.

### Intravenous Formulations

Intravenous administration also provides a route of administration where other routes such as oral, pulmonary and sublingual are not desired or suitable. The intravenous route of administration is particularly advantageous where irregular absorption is a concern. These formulations are prepared in accordance with the procedure used to prepare the above-mentioned nebulizer solutions. In certain embodiments, the formulations may also contain pH modifiers and or tonicity modifying agents to limit the irritation to the blood vessel upon administration. The formulations may then be transferred into a single or mult-dose stoppered vials and subsequently injecting with a needle and syringe. The dosage ranges will vary with the choice of cannabinoid. In certain embodiments, the dose will be adjusted to provide a dose that is therapeutically equivalent to the oral dose of Marinol. One of skill will appreciate that the dose will typically be lower than the dose delivered through other routes of administration as the intravenous route provides essentially complete bioavailability of the administered dose.

### Ophthalmic Preparations

Ophthalmic administration provides a route of administration where the intended action involves the ocular system. Ophthalmic formulations are prepared in accordance with the procedure described for preparing the above-mentioned nebulizer formulations. In certain embodiments, the ophthalmic preparations will also contain pH modifiers and or tonicity modifying agents in order to substantially prevention the irritation to the eye upon administration.

In certain other embodiments, the ophthalmic formulations are ointments. In certain other embodiments, the formulations contain lanolin, petrolatum, a high molecular weight glycol, e.g., PEG-400, mineral oil, or combinations thereof. In certain embodiments, the formulations further comprise water.

The ophthalmic formulation may then be transferred into single or mult-dose containers, made from pharmaceutically acceptable materials suitable for ophthalmic administration. The dosage ranges will vary with the choice of cannabinoid. The cannabinoid will be present in a concentration such that a dose will provide a therapeutically effective amount of cannabinoid to treat a condition of the eye, e.g., glaucoma.

In certain embodiments of the invention, the ophthalmic formulation contains dronabinol in a concentration of about 1% by weight. In certain other embodiments, the dosage provides from about 0.01 mg to about 10 mg dronabinol, preferably from about 0.5 mg to about 5 mg, and more preferably from about 1 mg to about 3 mg.

The invention is also directed to stable aqueous cannabinoid formulations for intravenous administration. In certain embodiments where the formulation contains dronabinol, the intravenous dose is from about 0.01 mg to about 50 mg.

In preferred embodiments, the cannabinoid formulations of the invention do not degrade to an unacceptable extent such that the final product (cannabinoid dosage form) has a shelf-life of at least about 2 years. As previously mentioned, this means that the active ingredient (e.g., dronabinol) within the dosage form remains within 90-110% of its initial amount in the dosage form during the desired (e.g., labeled) shelf-life of the dosage form (e.g., a minimum of 2 years after the date of manufacture of the dosage form). In further preferred embodiments, where the dosage form contains dronabinol as the active ingredient, the dosage form will contain not greater than 2% D8THC during the claimed shelf-life of the dosage form. In further preferred embodiments, where the dosage form contains dronabinol, the dosage form will contain not greater than 2% cannabidiol during the claimed shelf-life of the dosage form. In further preferred embodiments, where the dosage form contains dronabinol, the dosage form will contain not greater than 1% exo-THC. In certain especially preferred embodiments where the dosage form contains dronabinol as the active ingredient, the dosage form will contain the following during its claimed shelf-life: (i) not less than 90% of the initial dronabinol content; (ii) not greater than about 2% cannabinol; (iii) not greater than about 2% delta-8-THC; (iv) not greater than 2% cannabidiol; (v) not greater than about 0.5% exo-THC; or any combination of the foregoing. Although exo-THC is not a degradant of dronabinol, it is an impurity formed during the synthesis of dronabinol. These ranges of particular degradants/impurities may be applicable for other cannabinoids, as well.

It is believed that the aqueous cannabinoid formulations in accordance with the present invention are significantly more stable than the formulations in the art that describe limited amounts of water or exposure to water for limited periods of time during manufacture (e.g. Dedhiya, et al). The stability studies set forth in the appended examples are believed to confirm that by utilizing organic cosolvents as well as buffered aqueous medium, and optionally stabilizers, the cannabinoid drug product that is obtained is stable for at least about two years at room temperature.

### Route of Administration

The formulations of the present invention are preferably administered by the following routes: pulmonary, e.g., via nebulizer; orally, e.g. via oral syrup, sublingually, e.g., via a sublingual spray; intravenously; transdermally, e.g., via a topical gel and ophthalmically, e.g., via an ointment or liquid drop. However, one skilled in the art will appreciate that the stabilized aqueous cannabinoid formulations of the present invention are not limited to administration by these routes, and can be administered via the nasogastric route, an intramuscular route, or by direct absorption through mucous membrane tissues (e.g., buccally or rectally). Although formulations specifically suited to pulmonary, oral, sublingual, intravenous; transdermal, and ophthalmic administration are presently preferred, the compositions of the present invention can also be formulated for vaginal, rectal, parenteral or transmucosal administration. Thus, the dosage form can be a solution, suspension, emulsion, suppository, spray, aerosol, gel, drops, syrup, elixir, or other dosage form, as desired.

### Dosage

The oral dosage range of dronabinol or other cannabinoid may vary widely from 2.5 mg to 20 mg daily, in single or divided doses, or therapeutically equivalent amounts of one or more other cannabinoids may be utilized (as can be determined by one skilled in the art).

For other routes of administration, such as pulmonary, sublingual, intravenous and transdermal, wherein the therapeutic affect desired is similar to the therapeutic affect achieved with oral delivery, the dosage will vary to deliver an amount of cannabinoid that will be therapeutically equivalent to the desired oral dose.

The amount of cannabinoid present in the dosage will also vary in accordance with the particular cannabinoid potency upon administration, (e.g., higher potency upon delivery will require less cannabinoid).

### ADVANTAGES OF THE INVENTION

The branded product Marinol® (Dronabinol solution in soft gelatin capsules) is highly unstable at room temperature. Therefore the manufacturer of Marinol (Unimed Pharmaceuticals Inc.) recommends that the product be stored at refrigerated (2-8° C.) or cool (8-15° C.) conditions (Marinol package label, Physicians Desk Reference®, Ed. 2003). Also, aqueous cannabinoid formulations in the prior art are not considered stable when the aqueous component of the carrier exceeds about 20% v/v. At higher concentrations of water, the cannabinoid readily falls out of solution. Unlike the prior art cannabinoid formulations, the present invention provides an aqueous cannabinoid (e.g., dronabinol) formulation drug product that is preferably stable at all conditions—refrigerated, cool and room temperature (25° C./60% RH). Factors contributing to the improved stability, particularly at room temperature, of the present invention include: the use of a buffered aqueous system. In certain embodiments, additional factors contributing to improved stability of the cannabinoid dosage forms of the present invention include the addition of effective stabilizing amounts of organic bases (e.g., ethanolamine and meglumine); and/or the addition of additional effective stabilizing amounts of anti-oxidants (e.g., BHA, BHT, and sodium ascorbate).

In certain preferred embodiments, the cannabinoid formulations of the present invention may improve the delivery of the cannabinoid with respect to the extent, rate, and/or consistency of absorption from the location of administration.

### USES OF THE PRESENT INVENTION

The formulations of the present invention are useful in treatment and prevention of a very wide range of disorders, including, for example, nausea, vomiting, anorexia, cachexia, pain, gastrointestinal tract distress (such as heartburn, indigestion, stomachache, sour stomach), inflammatory bowel disease, Crohn's disease, gastritis, irritable bowel syndrome, ulcerative colitis, migraine headaches, postmenstrual syndrome, Alzheimer's dementia, agitation, muscle spasms and other involuntary movement disorders, Parkinson's disease and Parkinsonian-type symptoms, spasticity as result of multiple sclerosis, glaucoma and anxiety disorders. Cannabinoids such as dronabinol have also been reported as showing other biological activities which lend themselves to possible therapeutic applications, such as in the treatment of migraine headaches, spinal cord injury, anxiety, glaucoma and as an analgesic (e.g., to treat neuropathic pain). Cannabinoids such as dronabinol may be used together with opioid analgesics in a synergistic way to relieve pain; advantages of the combination may include decreased administration of opioids (leading to decreased side effects) and may be opioid-sparing (i.e., allowing for a reduced dose of opioid to achieve an equivalent effect). Dronabinol has also been used in the treatment of cancer cachexia (where the loss of appetite induces malnutrition in cancer patients). It has also been used to treat movement disorders including dystonia, Huntington's disease, Parkinson's disease and Tourette's syndrome; epilepsy, and for appetite stimulation in Alzheimer's disease. The use of cannabinoid formulations prepared in accordance with the present invention is contemplated for any and all of the above uses, and any other use known or which become known to those skilled in the art.

### DETAILED DESCRIPTION OF PREFERRED EMBODIMENTS

The following examples illustrate various aspects of the present invention, and are set forth to assist in understanding the invention. These examples should not be construed as specifically limiting the invention described and claimed herein. Variations of the invention, including the substitution of all equivalents now known or later developed, which would be within the purview of those skilled in the art, and changes in formulation or minor changes in experimental design, are considered to fall within the scope of the invention and appended claims.

The following equipment and procedure was used to develop and analyze room temperature stable aqueous dronabinol formulations:

### Example 1

### Manufacturing Procedure for Nebulizer Formulations

The investigational test compound Delta-9-THC was obtained. All other chemicals used in the formulations were of pharmaceutical grade.

### Equipment

### Agilent 1100 HPLC

### Mixer (IKA) or Vortexer

### Digital Hot-Plate Stirrer

### Glass Beakers

### Volumetric Flasks

### Glass Pipette with Rubber Bulb

### Glass Container

### Formulation Development—Delta-9-THC in Ethanol Stock Solution

Delta-9-tetrahydrocannabinol (dronabinol) is chemically synthesized as per procedures known to those skilled in the art, and is supplied as a light-yellow resinous oil that is sticky at room temperature and hardens upon refrigeration. Chemically synthesized dronabinol is supplied in a round bottom flask with high-vacuum adaptor with a 24/40 o-ring seal joint and bakeable PTFE plug.

Dronabinol in ethanol was prepared as follows: An oil bath (vacuum pump oil, Fisher CAS #72623-87-1) was heated to 90-95° C. A container containing the delta-9-THC was placed in the preheated oil bath for 10 min (after removing the vacuum adapter of the container containing D9-THC) until it turns into a flowable liquid. The weight of the empty glass container (W₁) was calculated. The D9-THC was transferred to the container by using a glass pipette.

The weight of the container with Delta-9-THC (W₂) was calculated. The exact amount of D9-THC transferred was calculated according to the following formula: (ΔW=W₂−W₁). The actual amount of D9-THC in the container was calculated according to the following formula: D9-THC=ΔW×Potency. The actual amount of ethanol to be added to the mixture to obtain the required concentration of delta-9-THC was calculated. Ethanol was then added and mixed well by a vortexer for about 5 minutes.

The aqueous formulations were then made by adding to the stock solution, polyethylene glycol, propylene glycol, and finally, water or buffer solution. The mixture was then mixed well by a vortexer for about 5 minutes.

The mixture was cooled to room temperature and a sample was submitted for analysis.

The flask containing D9-THC was removed from the oil bath. A vacuum adaptor was put on and the mixture was allowed to cool down for about an hour. After positioning the knob to the adapter to the open position, the flask containing the mixture was exposed to the vacuum for about 15 minutes and then the knob was closed. The flask containing the mixture was then stored in the refrigerator.

### Example 2

### Dronabinol Nebulizer Formulations with Buffer (pH 7.01)

The aqueous dronabinol formulations of Example 2 containing buffer solution (pH 7.10) were prepared according to the procedure described in Example 1. Formulations 2-A through 2-E each contained 5 mg/mL of dronabinol with varying volumetric amounts of ethanol, buffer solution, polyethylene glycol and propylene glycol as set forth in Table 1 below.

TABLE 1

| | Conc. of | | Buffer (pH | | |
|---|---|---|---|---|---|
| | THC | Ethanol | 7.01) | PEG | PG |
| Formulation # | (mg/mL) | % v/v | % v/v | % v/v | % v/v |
| 2-A | 5.00 | 35.0 | 35.0 | 20.0 | 10.0 |
| 2-B | 5.00 | 35.0 | 38.3 | 16.7 | 10.0 |
| 2-C | 5.00 | 40.0 | 35.0 | 15.0 | 10.0 |
| 2-D | 5.00 | 40.0 | 37.0 | 13.0 | 10.0 |
| 2-E | 5.00 | 30.0 | 35.0 | 25.0 | 10.0 |

### Example 3

### Dronabinol Nebulizer Formulations with Deionized Water

The aqueous dronabinol formulations of Example 3 containing deionized water were prepared in accordance with procedure described in Example 1 using deionized water. Formulations 3-A through 3-E each contained 5 mg/mL of dronabinol with varying volumetric amounts of ethanol, deionized water, polyethylene glycol and propylene glycol as set forth in Table 2 below.

TABLE 2

| | Conc. of | | | | |
|---|---|---|---|---|---|
| | THC | Ethanol | DI Water | PEG | PG |
| Formulation # | (mg/mL) | % v/v | % v/v | % v/v | % v/v |
| 3-A | 5.00 | 35.0 | 35.0 | 20.0 | 10.0 |
| 3-B | 5.00 | 35.0 | 38.3 | 16.7 | 10.0 |
| 3-C | 5.00 | 40.0 | 35.0 | 15.0 | 10.0 |
| 3-D | 5.00 | 40.0 | 37.0 | 13.0 | 10.0 |
| 3-E | 5.00 | 30.0 | 35.0 | 25.0 | 10.0 |

### Example 4

### Dronabinol Nebulizer Formulations with Buffer Solution and Anti-Oxidants

The aqueous dronabinol formulation 4-A was prepared in accordance with procedure described in Example 2. The measured concentration of dronabinol was 4.95 mg/mL, and consisted of volumetric amounts as follows: 35.0% ethanol, 38.3% buffer solution (pH 7.01), 16.7% polyethylene glycol and 10.0% propylene glycol. Formulations 4-B through 4-H were prepared in accordance with the formulation 4-A, but further contained an amount of an antioxidant described in Table 3 below.

The aqueous dronabinol formulation 4-I was prepared in accordance with procedure described in Example 2. The measured concentration of dronabinol was 4.95 mg/mL, and consisted of volumetric amounts as follows: 40.0% ethanol, 37.0% buffer solution (pH 7.01), 16.7% polyethylene glycol and 10.0% propylene glycol. Formulations 4-J through 4-P were prepared in accordance with the formulation 4-I, but further contained an amount of an antioxidant described in Table 3 below.

TABLE 3

| | | | Buffer | | | |
|---|---|---|---|---|---|---|
| | | | (pH | | | |
| | Conc. | Ethanol | 7.01) | PEG % | PG % | Antioxidant |
| Formulation # | (mg/mL) | % v/v | % v/v | v/v | v/v | % v/v |
| 4-A | 4.95 | 35.0 | 38.3 | 16.7 | 10.0 | — |
| 4-B | 4.95 | 35.0 | 38.3 | 16.7 | 10.0 | Ascorbic Palmitate |
| | | | | | | (0.1) |
| 4-C | 4.95 | 35.0 | 38.3 | 16.7 | 10.0 | BHA (0.01) |
| 4-D | 4.95 | 35.0 | 38.3 | 16.7 | 10.0 | BHT (0.01) |
| 4-E | 4.95 | 35.0 | 38.3 | 16.7 | 10.0 | Propyl Gallate (0.15) |
| 4-F | 4.95 | 35.0 | 38.3 | 16.7 | 10.0 | Sod Ascorbate (0.01) |
| 4-G | 4.95 | 35.0 | 38.3 | 16.7 | 10.0 | Tocopherol (0.05) |
| 4-H | 4.95 | 35.0 | 38.3 | 16.7 | 10.0 | MEA (0.5) |
| 4-I | 4.95 | 40.0 | 37.0 | 16.7 | 10.0 | — |
| 4-J | 4.95 | 40.0 | 37.0 | 16.7 | 10.0 | Ascorbic Palmitate |
| | | | | | | (0.1) |
| 4-K | 4.95 | 40.0 | 37.0 | 13.0 | 10.0 | BHA (0.01) |
| 4-L | 4.95 | 40.0 | 37.0 | 13.0 | 10.0 | BHT (0.01) |
| 4-M | 4.95 | 40.0 | 37.0 | 13.0 | 10.0 | Propyl Gallate (0.15) |
| 4-N | 4.95 | 40.0 | 37.0 | 13.0 | 10.0 | Sod Ascorbate (0.01) |
| 4-O | 4.95 | 40.0 | 37.0 | 13.0 | 10.0 | Tocopherol (0.05) |
| 4-P | 4.95 | 40.0 | 37.0 | 13.0 | 10.0 | MEA (0.5) |

### Example 5

### Dronabinol Nebulizer Formulations with Buffer Solution and Anti-Oxidants

The aqueous dronabinol formulation 5-A was prepared in accordance with procedure described in Example 2. The concentration of dronabinol was 5 mg/mL, and consisted of volumetric amounts as follows: 43% ethanol, 10% polyethylene glycol-400, 10% propylene glycol and 37% buffer solution (pH 7.01). Formulations 5-B through 5-E were prepared in accordance with the formulation 5-A, but further contained an amount of an antioxidant described in Table 4 below.

The aqueous dronabinol formulation 5-F was prepared in accordance with procedure described in Example 2. The concentration of dronabinol was 5 mg/mL, and consisted of volumetric amounts as follows: 50% ethanol, 5% polyethylene glycol-400, 10% propylene glycol and 35% buffer solution (pH 7.01). Formulations 5-G through 5-J were prepared in accordance with the formulation 5-F, but further contained an amount of an antioxidant described in Table 4 below.

The aqueous dronabinol formulation 5-K was prepared in accordance with procedure described in Example 2. The concentration of dronabinol was 5 mg/mL, and consisted of volumetric amounts as follows: 50% ethanol, 5% polyethylene glycol-400, 5% propylene glycol and 40% buffer solution (pH 7.01). Formulations 5-L through 5-O were prepared in accordance with the formulation 5-K, but further contained an amount of an antioxidant described in Table 4 below.

TABLE 4

| | | EtOH | PEG- | PG | Buffer | |
|---|---|---|---|---|---|---|
| | Conc. | % | 400 | % | pH 7.01 | Antioxidants % |
| Formulation # | (mg/mL) | v/v | % v/v | v/v | % v/v | v/v |
| 5-A | 5 | 43 | 10 | 10 | 37 | Control |
| 5-B | 5 | 43 | 10 | 10 | 37 | BHA (0.01%) |
| 5-C | 5 | 43 | 10 | 10 | 37 | BHT (0.01%) |
| 5-D | 5 | 43 | 10 | 10 | 37 | BHA + BHT |
| | | | | | | (0.05%) |
| 5-E | 5 | 40 | 13 | 10 | 37 | Na Ascorbate |
| | | | | | | (0.01%) |
| 5-F | 5 | 50 | 5 | 10 | 35 | Control |
| 5-G | 5 | 50 | 5 | 10 | 35 | BHA (0.01%) |
| 5-H | 5 | 50 | 5 | 10 | 35 | BHT (0.01%) |
| 5-I | 5 | 50 | 5 | 10 | 35 | BHA + BHT |
| | | | | | | (0.05%) |
| 5-J | 5 | 50 | 5 | 10 | 35 | Na Ascorbate |
| | | | | | | (0.01%) |
| 5-K | 5 | 50 | 5 | 5 | 40 | Control |
| 5-L | 5 | 50 | 5 | 5 | 40 | BHA (0.01%) |
| 5-M | 5 | 50 | 5 | 5 | 40 | BHT (0.01%) |
| 5-N | 5 | 50 | 5 | 5 | 40 | BHA + BHT |
| | | | | | | (0.05%) |
| 5-O | 5 | 50 | 5 | 5 | 40 | Na Ascorbate |
| | | | | | | (0.01%) |

### Example 6

### Stability Studies

The International Conference on Harmonisation (ICH) of Technical Requirements for Registration of Pharmaceuticals for Human Use (ICH) has adopted scientific standards for stability testing. These standards are the basis of regulatory guidances published by the FDA (Guidance for Industry, Q1A). The purpose of stability tests is to provide evidence on how the quality and safety of the product varies with time. As per the FDA guidance, a finished product is assigned a tentative two-year expiration dating at room temperature (25° C.) if the product is stable, for 6 months at accelerated testing conditions, 40° C. (15° C. above the designated long-term storage temperature). In Example 6, the aqueous dronabinol formulations of Examples 2 through 5 were analyzed at zero time.

The concentrations of dronabinol and the impurities/degradants [Cannabidiol (CBD), Cannabinol (CBN) and D8THC] were determined by using the compendial (USP) HPLC method.

The concentrations of dronabinol and the impurities/degradants [Cannabidiol (CBD), Cannabinol (CBN) and D8THC] of formulations 2-A through 2-E at zero time are listed in Table 5 below.

TABLE 5

| | | | | | | Total | |
|---|---|---|---|---|---|---|---|
| | Conc. of | | | | D8- | impurities (%) | |
| Formulation | THC | Potency | CBD | CBN | THC | | % |
| # | (mg/mL) | % | % | % | % | % | Increase |
| 2-A | 4.87 | 98.32 | 0.16 | 0.61 | 0.45 | 3.37 | 0.00 |
| 2-B | 4.88 | 98.58 | 0.17 | 0.53 | 0.44 | 3.27 | 0.00 |
| 2-C | 4.88 | 98.49 | 0.15 | 0.49 | 0.43 | 3.11 | 0.00 |
| 2-D | 4.89 | 98.80 | 0.15 | 0.49 | 0.38 | 2.99 | 0.00 |
| 2-E | 4.91 | 99.10 | 0.16 | 0.46 | 0.51 | 3.13 | 0.00 |

The concentrations of dronabinol and the impurities/degradants [Cannabidiol (CBD), Cannabinol (CBN) and D8THC] of formulations 3-A through 3-E at zero time are listed in Table 6 below.

TABLE 6

| | | | | | | Total | |
|---|---|---|---|---|---|---|---|
| | Conc. of | | | | D8- | Impurities (%) | |
| Formulation | THC | Potency | CBD | CBN | THC | | % |
| # | (mg/mL) | % | % | % | % | % | Increase |
| 3-A | 4.93 | 99.48 | 0.17 | 0.46 | 0.50 | 3.19 | 0.00 |
| 3-B | 4.98 | 100.57 | 0.15 | 0.48 | 0.46 | 3.04 | 0.00 |
| 3-C | 4.97 | 100.29 | 0.15 | 0.50 | 0.41 | 2.99 | 0.00 |
| 3-D | 4.96 | 100.06 | 0.18 | 0.46 | 0.49 | 3.20 | 0.00 |
| 3-E | 4.84 | 97.80 | 0.13 | 0.50 | 0.43 | 2.99 | 0.00 |

The concentrations of dronabinol and the impurities/degradants [Cannabidiol (CBD), Cannabinol (CBN) and D8THC] of formulations 4-A through 4-O at zero time are listed in Table 7 below.

TABLE 7

| | Potency | CBD | | D8- | Total Impurities (%) | |
|---|---|---|---|---|---|---|
| Formulation # | % | % | CBN % | THC % | % | % Increase |
| 4-A | 100 | 0.21 | 0.55 | 0.55 | 4.17 | 0 |
| 4-B | 100 | 0.2 | 0.49 | 0.45 | 3.69 | 0 |
| 4-C | 100 | 0.07 | 0.46 | 0.55 | 3.40 | 0 |
| 4-D | 100 | 0.07 | 0.45 | 0.61 | 5.01 | 0 |
| 4-E | 100 | 0.07 | 0.49 | 0.62 | 3.64 | 0 |
| 4-F | 100 | 0.07 | 0.47 | 0.61 | 3.67 | 0 |
| 4-G | 100 | 0.12 | 0.34 | 0.60 | 3.84 | 0 |
| 4-H | 100 | 0.07 | 0.47 | 0.64 | 4.07 | 0 |
| 4-I | 100 | 0.21 | 0.52 | 0.53 | 3.92 | 0 |
| 4-J | 100 | 0.29 | 0.54 | 0.48 | 3.5 | 0 |
| 4-K | 100 | 0.10 | 0.46 | 0.64 | 4.25 | 0 |
| 4-L | 100 | 0.08 | 0.46 | 0.59 | 5.04 | 0 |
| 4-M | 100 | 0.09 | 0.53 | 0.62 | 3.87 | 0 |
| 4-N | 100 | 0.08 | 0.55 | 0.59 | 3.93 | 0 |
| 4-O | 100 | 0.11 | 0.49 | 0.60 | 4.14 | 0 |
| 4-P | 100 | 0.07 | 0.44 | 0.55 | 4.10 | 0 |

The concentrations of dronabinol and the impurities/degradants [Cannabidiol (CBD), Cannabinol (CBN) and D8THC] of formulations 5-A through 5-O at zero time are listed in Table 8 below.

TABLE 8

| | | | | | Total | |
|---|---|---|---|---|---|---|
| | | | | | Impurities % | |
| | | CBD | CBN | | | % |
| Formulation # | Potency % | % | % | d8-THC % | % | Increase |
| 5-A | 100.00 | 0.26 | 0.52 | 0.46 | 3.67 | 0.00 |
| 5-B | 100.00 | 0.26 | 0.46 | 0.54 | 4.09 | 0.00 |
| 5-C | 100.00 | 0.26 | 0.46 | 0.57 | 3.76 | 0.00 |
| 5-D | 100.00 | 0.26 | 0.46 | 0.58 | 3.93 | 0.00 |
| 5-E | 100.00 | 0.26 | 0.46 | 0.59 | 3.89 | 0.00 |
| 5-F | 100.00 | 0.27 | 0.44 | 0.57 | 4.09 | 0.00 |
| 5-G | 100.00 | 0.26 | 0.46 | 0.59 | 3.81 | 0.00 |
| 5-H | 100.00 | 0.26 | 0.47 | 0.59 | 3.92 | 0.00 |
| 5-I | 100.00 | 0.26 | 0.47 | 0.59 | 3.70 | 0.00 |
| 5-J | 100.00 | 0.25 | 0.46 | 0.57 | 4.32 | 0.00 |
| 5-K | 100.00 | 0.28 | 0.44 | 0.55 | 4.34 | 0.00 |
| 5-L | 100.00 | 0.30 | 0.47 | 0.59 | 3.81 | 0.00 |
| 5-M | 100.00 | 0.30 | 0.45 | 0.56 | 3.83 | 0.00 |
| 5-N | 100.00 | 0.30 | 0.46 | 0.58 | 3.94 | 0.00 |
| 5-O | 100.00 | 0.30 | 0.46 | 0.55 | 3.74 | 0.00 |

### Example 7

In Example 7, the aqueous dronabinol formulations 2-A through 2-E, formulations 3-A through 3-E, formulations 4-B through 4-P and formulations 5-A through 5-O were stored at 40° C. for one month and analyzed in accordance with Example 6.

The concentrations of dronabinol and the impurities/degradants [Cannabidiol (CBD), Cannabinol (CBN) and D8THC] of formulations 2-A through 2-E after storage at 40° C. for one month are listed in Table 9 below.

TABLE 9

| | Conc. | | | | | Total | |
|---|---|---|---|---|---|---|---|
| | of | | | | | impurities | |
| | THC | | | | | (%) | |
| | (mg/ | Potency | CBD | CBN | D8- | | % |
| Formulation # | mL) | % | % | % | THC % | % | Increase |
| 2-A | 4.59 | 94.16 | 0.05 | 0.65 | 0.46 | 4.26 | 0.89 |
| 2-B | 4.58 | 96.88 | 0.04 | 0.54 | 0.45 | 5.78 | 1.61 |
| 2-C | 4.44 | 91.03 | 0.09 | 0.60 | 0.52 | 4.51 | 1.40 |
| 2-D | 4.58 | 94.80 | 0.02 | 0.53 | 0.48 | 5.44 | 1.52 |
| 2-E | 4.33 | 88.15 | 0.03 | 0.58 | 0.42 | 4.00 | 0.87 |

The concentrations of dronabinol and the impurities/degradants [Cannabidiol (CBD), Cannabinol (CBN) and D8THC] of formulations 2-A through 2-E after storage at 40° C. for one month are listed in Table 10 below.

TABLE 10

| | | | | | | Total | |
|---|---|---|---|---|---|---|---|
| | | | | | | impurities | |
| | Conc. of | | | | D8- | (%) | |
| Formulation | THC | Potency | CBD | CBN | THC | | % |
| # | (mg/mL) | % | % | % | % | % | Increase |
| 3-A | 4.31 | 87.38 | 0.07 | 0.60 | 0.43 | 5.40 | 2.21 |
| 3-B | 4.47 | 89.76 | 0.07 | 0.61 | 0.53 | 5.70 | 2.66 |
| 3-C | 4.38 | 88.16 | 0.08 | 0.58 | 0.43 | 5.39 | 2.40 |
| 3-D | 4.41 | 89.05 | 0.09 | 0.58 | 0.39 | 5.71 | 2.51 |
| 3-E | 4.36 | 90.06 | 0.09 | 0.64 | 0.46 | 6.93 | 3.94 |

The concentrations of dronabinol and the impurities/degradants [Cannabidiol (CBD), Cannabinol (CBN) and D8THC] of formulations 4-B through 4-H and 4-J through 4-P after storage at 40° C. for one month are listed in Table 11 below.

TABLE 11

| | | | | | Total | |
|---|---|---|---|---|---|---|
| | | | | | Impurities (%) | |
| | Potency | CBD | | | | % |
| Formulation # | % | % | CBN % | D8-THC % | % | Increase |
| 4-B | 101.95 | 0.05 | 1.86 | 0.49 | 11.27 | 7.58 |
| 4-C | 96.20 | 0.05 | 0.44 | 0.57 | 4.82 | 1.42 |
| 4-D | 95.44 | 0.03 | 0.44 | 0.56 | 5.61 | 0.60 |
| 4-E | 88.84 | 0.02 | 0.81 | 0.53 | 10.66 | 7.02 |
| 4-F | 94.59 | 0.03 | 0.47 | 0.57 | 4.99 | 1.32 |
| 4-G | 76.14 | 0.50 | 0.92 | 0.53 | 21.26 | 17.42 |
| 4-H | 94.07 | 0.01 | 0.45 | 0.53 | 5.42 | 1.35 |
| 4-J | 96.86 | 0.05 | 0.96 | 0.55 | 7.80 | 4.30 |
| 4-K | 100.91 | 0.03 | 0.44 | 0.56 | 4.63 | 0.38 |
| 4-L | 98.26 | 0.02 | 0.44 | 0.52 | 5.54 | 0.50 |
| 4-M | 92.21 | 0.02 | 0.84 | 0.56 | 10.70 | 6.83 |
| 4-N | 96.19 | 0.02 | 0.46 | 0.52 | 4.72 | 0.79 |
| 4-O | 83.76 | 0.35 | 0.73 | 0.57 | 16.55 | 12.41 |
| 4-P | 95.50 | 0.01 | 0.45 | 0.53 | 5.82 | 1.72 |

The concentrations of dronabinol and the impurities/degradants [Cannabidiol (CBD), Cannabinol (CBN) and D8THC] of formulations 5-A through 5-P after storage at 40° C. for one month are listed in Table 12 below.

TABLE 12

| | | | | | Total | |
|---|---|---|---|---|---|---|
| | | | | | Impurities % | |
| | | CBD | CBN | | | % |
| Formulation # | Potency % | % | % | d8-THC % | % | increase |
| 5-A | 97.79 | 0.04 | 0.63 | 0.57 | 6.65 | 2.98 |
| 5-B | 96.27 | 0.17 | 0.45 | 0.55 | 4.74 | 0.65 |
| 5-C | 96.19 | 0.07 | 0.45 | 0.57 | 5.23 | 1.47 |
| 5-D | 98.92 | 0.03 | 0.46 | 0.59 | 4.78 | 0.85 |
| 5-E | 99.52 | 0.03 | 0.47 | 0.55 | 5.72 | 1.83 |
| 5-F | 95.96 | 0.02 | 0.46 | 0.60 | 5.10 | 1.01 |
| 5-G | 98.81 | 0.04 | 0.45 | 0.63 | 5.36 | 1.55 |
| 5-H | 97.45 | 0.02 | 0.46 | 0.57 | 4.74 | 0.82 |
| 5-I | 96.69 | 0.03 | 0.48 | 0.56 | 4.73 | 1.03 |
| 5-J | 99.22 | 0.03 | 0.48 | 0.57 | 5.49 | 1.17 |
| 5-K | 98.34 | 0.04 | 0.48 | 0.59 | 5.57 | 1.23 |
| 5-L | 96.03 | 0.04 | 0.47 | 0.58 | 5.30 | 1.49 |
| 5-M | 97.47 | 0.04 | 0.46 | 0.61 | 5.46 | 1.63 |
| 5-N | 99.67 | 0.05 | 0.48 | 0.57 | 5.40 | 1.46 |
| 5-O | 99.05 | 0.04 | 0.49 | 0.56 | 5.69 | 1.95 |

### Example 8

In Example 10, the aqueous dronabinol formulations 2-A through 2-E, formulations 3-A through 3-E, formulations 4-B through 4-H and H-J through 4-P were stored at 40° C. for two months and analyzed in accordance with Example 6.

The concentrations of dronabinol and the impurities/degradants [Cannabidiol (CBD), Cannabinol (CBN) and D8THC] of formulations 2-A through 2-E after storage at 40° C. for two months are listed in Table 13 below.

TABLE 13

| | | | | | | Total | |
|---|---|---|---|---|---|---|---|
| | | | | | | impurities | |
| | Conc. of | | | | D8- | (%) | |
| Formulation | THC | Potency | CBD | CBN | THC | | % |
| # | (mg/mL) | % | % | % | % | % | Increase |
| 2-A | 4.69 | 96.27 | 0.09 | 0.75 | 0.42 | 7.81 | 4.44 |
| 2-B | 4.64 | 94.98 | 0.09 | 0.71 | 0.51 | 5.82 | 2.55 |
| 2-C | 4.74 | 97.08 | 0.08 | 0.65 | 0.44 | 5.41 | 2.30 |
| 2-D | 4.68 | 95.63 | 0.08 | 0.62 | 0.50 | 5.51 | 2.52 |
| 2-E | 4.62 | 94.22 | 0.11 | 0.64 | 0.47 | 5.50 | 2.37 |

The concentrations of dronabinol and the impurities/degradants [Cannabidiol (CBD), Cannabinol (CBN) and D8THC] of formulations 3-A through 3-E after storage at 40° C. for two months are listed in Table 14 below.

TABLE 14

| | | | | | | Total | |
|---|---|---|---|---|---|---|---|
| | | | | | | impurities | |
| | Conc. of | | | | D8- | (%) | |
| Formulation | THC | Potency | CBD | CBN | THC | | % |
| # | (mg/mL) | % | % | % | % | % | Increase |
| 3-A | 4.56 | 92.51 | 0.07 | 0.88 | 0.42 | 7.64 | 4.45 |
| 3-B | 4.62 | 92.75 | 0.08 | 0.82 | 0.50 | 7.55 | 4.51 |
| 3-C | 4.69 | 94.49 | 0.07 | 0.69 | 0.49 | 7.02 | 4.03 |
| 3-D | 4.55 | 91.85 | 0.08 | 0.69 | 0.48 | 7.38 | 4.18 |
| 3-E | 4.46 | 82.14 | 0.09 | 0.84 | 0.46 | 8.43 | 5.44 |

The concentrations of dronabinol and the impurities/degradants [Cannabidiol (CBD), Cannabinol (CBN) and D8THC] of formulations 4-B through 4-H and 4-J through 4-P after storage at 40° C. for two months are listed in Table 15 below.

TABLE 15

| | | | | | Total | |
|---|---|---|---|---|---|---|
| | | | | | Impurities (%) | |
| | Potency | CBD | | | | % |
| Formulation # | % | % | CBN % | D8-THC % | % | Increase |
| 4-B | 84.78 | 0.17 | 6.02 | 0.49 | 26.89 | 23.20 |
| 4-C | 98.34 | 0.09 | 0.5 | 0.59 | 6.37 | 2.97 |
| 4-D | 98.82 | 0.07 | 0.48 | 0.59 | 6.97 | 1.96 |
| 4-E | 90.66 | 0.43 | 1.45 | 0.46 | 14.09 | 10.45 |
| 4-F | 99.14 | 0.07 | 0.48 | 0.61 | 6.42 | 2.75 |
| 4-G | 66.64 | 0.56 | 1.68 | 0.55 | 32.33 | 28.49 |
| 4-H | 95.24 | 0.02 | 0.46 | 0.58 | 7.53 | 3.46 |
| 4-J | 85.83 | 0.14 | 2.59 | 0.48 | 16.34 | 12.84 |
| 4-K | 99.75 | 0.05 | 0.48 | 0.58 | 5.63 | 1.38 |
| 4-L | 101.24 | 0.04 | 0.47 | 0.56 | 6.66 | 1.62 |
| 4-M | 91.86 | 0.43 | 1.43 | 0.56 | 13.9 | 10.03 |
| 4-N | 98.88 | 0.03 | 0.47 | 0.61 | 6.24 | 2.31 |
| 4-O | 76.21 | 0.48 | 1.1 | 0.6 | 24.18 | 20.04 |
| 4-P | 97 | 0.01 | 0.46 | 0.57 | 8.08 | 3.98 |

### Example 9

In Example 9, the aqueous dronabinol formulations 2-A through 2-E, formulations 3-A through 3-E, formulations 4-C through 4-H and H-K through 4-P were stored at 40° C. for three months and analyzed in accordance with Example 6.

The concentrations of dronabinol and the impurities/degradants [Cannabidiol (CBD), Cannabinol (CBN) and D8THC] of formulations 2-A through 2-E after storage at 40° C. for three months are listed in Table 16 below.

TABLE 16

| | | | | | | Total | |
|---|---|---|---|---|---|---|---|
| | | | | | | impurities | |
| | Conc. of | | | | D8- | (%) | |
| Formulation | THC | Potency | CBD | CBN | THC | | % |
| # | (mg/mL) | % | % | % | % | % | Increase |
| 2-A | 4.53 | 93.12 | 0.13 | 0.87 | 0.47 | 7.74 | 4.37 |
| 2-B | 4.50 | 92.18 | 0.13 | 0.80 | 0.51 | 6.51 | 3.24 |
| 2-C | 4.72 | 96.85 | 0.12 | 0.67 | 0.35 | 5.89 | 2.78 |
| 2-D | 4.67 | 95.48 | 0.12 | 0.68 | 0.48 | 6.12 | 3.13 |
| 2-E | 4.62 | 94.09 | 0.12 | 0.68 | 0.34 | 5.80 | 2.67 |

The concentrations of dronabinol and the impurities/degradants [Cannabidiol (CBD), Cannabinol (CBN) and D8THC] of formulations 3-A through 3-E after storage at 40° C. for three months are listed in Table 17 below.

TABLE 17

| | | | | | | Total | |
|---|---|---|---|---|---|---|---|
| | | | | | | impurities | |
| | Conc. of | | | | D8- | (%) | |
| Formulation | THC | Potency | CBD | CBN | THC | | % |
| # | (mg/mL) | % | % | % | % | % | Increase |
| 3-A | 4.36 | 88.40 | 0.09 | 0.94 | 0.35 | 9.03 | 5.84 |
| 3-B | 4.39 | 88.04 | 0.11 | 1.11 | 0.50 | 9.76 | 6.72 |
| 3-C | 4.59 | 92.33 | 0.08 | 0.87 | 0.48 | 8.54 | 5.55 |
| 3-D | 4.40 | 88.77 | 0.09 | 0.90 | 0.50 | 9.57 | 6.37 |
| 3-E | 4.33 | 89.33 | 0.09 | 1.15 | 0.50 | 10.70 | 7.71 |

The concentrations of dronabinol and the impurities/degradants [Cannabidiol (CBD), Cannabinol (CBN) and D8THC] of formulations 4-C through 4-H and 4-K through 4-P after storage at 40° C. for three months are listed in Table 18 below. Stability testing of formulations 4-B and 4-J were discontinued due to precipitation.

TABLE 18

| Formulation | Potency | | CBN | D8-THC | Total Impurities (%) | |
|---|---|---|---|---|---|---|
| # | % | CBD % | % | % | % | % Increase |
| 4-C | 93.55 | 0.24 | 0.53 | 0.6 | 9.49 | 6.09 |
| 4-D | 95.72 | 0.2 | 0.52 | 0.57 | 9.53 | 4.52 |
| 4-E | 86.05 | 0.65 | 2.08 | 0.56 | 17.98 | 14.34 |
| 4-F | 91.36 | 0.14 | 0.57 | 0.57 | 8.85 | 5.18 |
| 4-G | 47.75 | 0.61 | 2.44 | 0.51 | 45.83 | 41.99 |
| 4-H | 85.66 | 0.04 | 0.51 | 0.53 | 10.64 | 6.57 |
| 4-K | 99.66 | 0.06 | 0.48 | 0.6 | 6.81 | 2.56 |
| 4-L | 93.6 | 0.08 | 0.5 | 0.6 | 8.48 | 3.44 |
| 4-M | 87.1 | 0.6 | 1.93 | 0.52 | 17.1 | 13.23 |
| 4-N | 98.75 | 0.05 | 0.49 | 0.61 | 7.56 | 3.63 |
| 4-O | 62.44 | 0.54 | 1.58 | 0.54 | 33.7 | 29.56 |
| 4-P | 92.25 | 0.03 | 0.47 | 0.55 | 10.34 | 6.24 |

### Example 10

In Example 10, the aqueous dronabinol formulations 2-A through 2-E were stored at 25° C. for thirteen months and analyzed in accordance with Example 6. The stability testing of formulations 3-A through 3-E was discontinued due to degradation and color change.

The concentrations of dronabinol and the impurities/degradants [Cannabidiol (CBD), Cannabinol (CBN) and D8THC] of formulations 2-A through 2-E after storage at 25° C. for thirteen months are listed in Table 19 below.

TABLE 19

| | | | | | | Total | |
|---|---|---|---|---|---|---|---|
| | | | | | | impurities | |
| | Conc. of | | | | D8- | (%) | |
| Formulation | THC | Potency | CBD | CBN | THC | | % |
| # | (mg/mL) | % | % | % | % | % | Increase |
| 2-A | 4.75 | 96.57 | 0.1 | 0.5 | 0.54 | 8.62 | 4.72 |
| 2-B | 3.31 | 70.88 | 0.84 | 1.09 | 0.51 | 26.28 | 22.11 |
| 2-C | 4.86 | 96.91 | 0.07 | 0.49 | 0.58 | 8.13 | 4.32 |
| 2-D | 4.72 | 96.20 | 0.08 | 0.62 | 0.46 | 9.65 | 5.73 |
| 2-E | 4.60 | 94.59 | 0.18 | 0.51 | 0.52 | 9.48 | 5.38 |

### Example 11

In Example 13, the aqueous dronabinol formulations 2-A and 2-C through 2-E were stored at 40° C. for thirteen months and analyzed in accordance with Example 6. The stability testing of formulation 2-B was stopped due to precipitation. The stability testing for formulations 3-A through 3-E was stopped due to degradation and color change.

The concentrations of dronabinol and the impurities/degradants [Cannabidiol (CBD), Cannabinol (CBN) and D8THC] of formulations 2-A and 2-2-C through 2-E after storage at 40° C. for three months are listed in Table 20 below.

TABLE 20

| | | | | | | Total | |
|---|---|---|---|---|---|---|---|
| | | | | | | impurities | |
| | Conc. of | | | | D8- | (%) | |
| Formulation | THC | Potency | CBD | CBN | THC | | % |
| # | (mg/mL) | % | % | % | % | % | Increase |
| 2-A | 3.93 | 79.81 | 0.19 | 0.81 | 0.52 | 20 | 16.1 |
| 2-C | 4.13 | 82.39 | 0.13 | 0.78 | 0.5 | 18.65 | 14.84 |
| 2-D | 3.76 | 76.59 | 0.18 | 1.07 | 0.45 | 23.46 | 19.54 |
| 2-E | 3.87 | 79.66 | 0.23 | 0.82 | 0.56 | 21.33 | 17.23 |

### Example 12

In Example 14, the aqueous dronabinol formulations 4-E and 4-M and formulations 5-A through 5-O were stored at 40° C. for eight months and analyzed in accordance with Example 6. The stability testing of formulations 4-B through 4-D, 4-F through 4-H, 4-J through 4-L, and 4-N through 4-P was stopped due to precipitation.

The concentrations of dronabinol and the impurities/degradants [Cannibidiol (CBD), Cannabinol (CBN) and D8THC] of formulations 4-E and 4-M after storage at 40° C. for eight months are listed in Table 21 below.

TABLE 21

| | | | | | Total Impurities | |
|---|---|---|---|---|---|---|
| | Potency | | | D8-THC | | Increase, |
| Formulation | % | CBD % | CBN % | % | % | + % |
| 4-E | 51.34 | 0.11 | 11.1 | 0.42 | 50.73 | 47.09 |
| 4-M | 59.39 | 0.06 | 8.97 | 0.58 | 42.66 | 38.79 |

The concentrations of dronabinol and the impurities/degradants [Cannibidiol (CBD), Cannabinol (CBN) and D8THC] of formulations 5-A through 5-O after storage at 40° C. for eight months are listed in Table 22 below.

TABLE 22

| | | | | | Total | |
|---|---|---|---|---|---|---|
| | | | | | Impurities % | |
| Formulation | Potency | | | | | % |
| # | % | CBD % | CBN % | d8-THC % | % | increase |
| 5-A | 89.8 | 0.43 | 0.65 | 0.55 | 14.06 | 10.39 |
| 5-B | 84.54 | 0.46 | 0.7 | 0.63 | 15.14 | 11.05 |
| 5-C | 83.96 | 0.56 | 0.75 | 0.55 | 19.22 | 15.46 |
| 5-D | 81.67 | 0.64 | 0.76 | 0.56 | 19.24 | 15.31 |
| 5-E | 91.04 | 0.59 | 0.7 | 0.63 | 15.69 | 11.8 |
| 5-F | 91.99 | 0.42 | 0.62 | 0.6 | 13.18 | 9.09 |
| 5-G | 88.65 | 0.43 | 0.61 | 0.6 | 12.96 | 9.15 |
| 5-H | 86.94 | 0.37 | 0.62 | 0.6 | 14.46 | 10.54 |
| 5-I | 81.84 | 0.48 | 0.71 | 0.56 | 16.68 | 12.98 |
| 5-J | 90.31 | 0.38 | 0.64 | 0.67 | 13.24 | 8.92 |
| 5-K | 87.51 | 0.55 | 0.71 | 0.53 | 15.04 | 10.7 |
| 5-L | 85.47 | 0.5 | 0.72 | 0.55 | 14.62 | 10.81 |
| 5-M | 77.82 | 0.72 | 0.88 | 0.56 | 22.09 | 18.26 |
| 5-N | 72.56 | 1.01 | 1.15 | 0.57 | 27.15 | 23.21 |
| 5-O | 84.71 | 0.65 | 0.82 | 0.55 | 17.98 | 14.24 |

The above stability studies show that formulations with buffer are more physically stable than formulations with water. Ethanol is extremely helpful in increasing the solubility of Delta-9-THC in aqueous solution. The physical stability of formulations can be increased by increasing the alcohol concentration. Formulations containing higher concentrations of polyethylene glycol have shown better stability, however, the viscosity of solution increased proportionally with PEG concentrations. These results prompted continued studies with an optimized ethanol concentrations of 35% with 16.7% PEG and 40% EtOH with 13% PEG as control formulations.

The above stability studies also show that the anti-oxidants BHA, BHT and sodium ascorbate are helpful in stabilizing Delta-9-THC in aqueous formulations (Table 15). Formulations containing anti-oxidants tocopherol and propyl gallate are highly unstable and degrade Delta-9-THC.

The factors that are considered for obtaining optimized nebulizer formulations include: physical stability, maximum permeation, bioavailability, viscosity and fine droplets.

### Example 13

### Manufacturing Procedure for Oral Syrup Formulations

The investigational test compound Delta-9-THC was obtained. All other chemicals used in the formulations were of pharmaceutical grade.

### Equipment

### Agilent 1100 HPLC

### Mixer (IKA) or Vortexer

### Digital Hot-Plate Stirrer

### Glass Beakers

### Volumetric Flasks

### Glass Pipette with Rubber Bulb

### Glass Container

### Formulation Development—Delta-9-THC in Ethanol

Delta-9-tetrahydrocannabinol (dronabinol) is chemically synthesized as per procedures known to those skilled in the art, and is supplied as a light-yellow resinous oil that is sticky at room temperature and hardens upon refrigeration. Chemically synthesized dronabinol is supplied in a round bottom flask with high-vacuum adaptor with a 24/40 o-ring seal joint and bakeable PTFE plug.

Dronabinol in ethanol was prepared as follows: An oil bath (vacuum pump oil, Fisher CAS #72623-87-1) was heated to 90-95° C. A container containing the delta-9-THC was placed in the preheated oil bath for 10 min (after removing the vacuum adapter of the container containing D9-THC) until it turns into a flowable liquid. The weight of the empty glass container (W₁) was calculated. The D9-THC was transferred to the container by using a glass pipette.

The weight of the container with Delta-9-THC (W₂) was calculated. The exact amount of D9-THC transferred was calculated according to the following formula: (ΔW=W₂−W₁). The actual amount of D9-THC in the container was calculated according to the following formula: D9-THC=ΔW×Potency. The actual amount of ethanol to be added to the mixture to obtain the required concentration of delta-9-THC was calculated. Ethanol was then added and mixed well by a vortexer for about 5 minutes.

The aqueous formulations were then made by adding to the stock solution, propylene glycol and water or buffer solution. The mixture was then mixed well by a vortexer for about 5 minutes.

The mixture was cooled to room temperature and a sample was submitted for analysis.

The flask containing D9-THC was removed from the oil bath. A vacuum adaptor was put on and the mixture was allowed to cool down for about an hour. After positioning the knob to the adapter to the open position, the flask containing the mixture was exposed to the vacuum for about 15 minutes and then the knob was closed. The flask containing the mixture was then stored in the refrigerator.

### Stability Studies

Several Dronabinol syrup aqueous formulations with ethanolic solutions of Delta-9-THC have been developed in the laboratory. Since Delta-9-THC is not soluble in water, ethanol and propylene glycol are used as cosolvents. The formulations were screened based on their physical stability with different sugars added. Stable formulations with optimum conditions were assessed for further studies.

### Example 14

### Dronabinol Oral Syrup Control Formulations

To determine the effects of different sugars, the aqueous dronabinol control formulations of Example 14 were prepared according to the procedure described in Example 13. Formulations 14-A contained 5.01 mg/mL of dronabinol and 14-B contained 9.83 mg/mL of dronabinol. These formulations also contained varying volumetric amounts of ethanol, propylene glycol and buffer solution (pH 7.01) as set forth in Table 23 below.

TABLE 23

| | Conc. of | | | Buffer | | |
|---|---|---|---|---|---|---|
| Formulation | THC | Ethanol | PG | (pH 7.01) | Sugar | |
| # | (mg/mL) | % w/w | % w/w | % w/w | Type | % w/w |
| 14-A | 5.01 | 50.48 | 5.22 | 44.30 | — | 0 |
| 14-B | 9.83 | 63.38 | 5.10 | 31.51 | — | 0 |

### Example 15

### Dronabinol Oral Syrup Formulations

The aqueous dronabinol formulations of Example 15 were prepared in accordance with procedure described in Example 14. Formulations 15-A and 15-B were control formulations that did not contain sugar. Formulations 15-C through 15-L contained sugars. The concentrations of dronabinol and amounts of other components in the formulations of Example 15 are set forth in Table 24 below.

TABLE 24

| | | | | Buffer | | |
|---|---|---|---|---|---|---|
| | Conc. of | | PG | (pH | | |
| Formulation | THC | Ethanol | % | 7.01) | Sugar | |
| # | (mg/mL) | % w/w | w/w | % w/w | Type | % w/w |
| 15-A | 4.74 | 46.37 | 5.25 | 43.39 | Sucrose | 4.99 |
| 15-B | 4.91 | 50.88 | 5.09 | 42.01 | Sucrose | 2.02 |
| 15-C | 5.05 | 49.72 | 5.21 | 43.02 | Sucrose | 2.04 |
| 15-D | 5.01 | 48.38 | 5.29 | 41.32 | Sucrose | 5.0 |
| 15-E | 5.11 | 49.90 | 5.35 | 42.73 | Sorbitol | 2.02 |
| 15-F | 4.97 | 48.56 | 5.19 | 41.14 | Sorbitol | 5.11 |
| 15-G | 10.05 | 56.94 | 5.31 | 35.54 | Sorbitol | 2.2 |
| 15-H | 9.91 | 59.38 | 5.17 | 30.46 | Sorbitol | 5.0 |
| 15-I | 9.93 | 56.62 | 5.26 | 35.93 | Sucrose | 2.20 |
| 15-J | 9.93 | 54.82 | 5.25 | 34.88 | Sucrose | 5.05 |
| 15-K | 10.03 | 56.61 | 5.28 | 35.89 | Fructose | 2.22 |
| 15-L | 9.85 | 54.03 | 5.19 | 35.80 | Fructose | 4.98 |

### Example 16

### Stability Studies

In Example 16, stability studies for the above formulations were performed in accordance with procedure described in Example 6 above. The aqueous dronabinol formulations 14A, 14-B and 15-A through 15-L were analyzed at zero time.

The concentrations of dronabinol and the impurities/degradants [Cannabidiol (CBD), Cannabinol (CBN) and D8THC] were determined by using the compendial (USP) HPLC method.

The concentrations of dronabinol and the impurities/degradants [Cannabidiol (CBD), Cannabinol (CBN) and D8THC] of formulations 14-A, 14-B and 15-A through 15-L at zero time are listed in Table 25 below.

TABLE 25

| | | | | | | Total | |
|---|---|---|---|---|---|---|---|
| | Conc. of | | | | D8- | Impurities | |
| Formulation | THC | Potency | CBD | CBN | THC | (%) | |
| # | (mg/mL) | % | % | % | % | % | % |
| 14-A | 5.01 | 100.00 | 0.09 | 0.87 | 0.62 | 4.91 | 0.00 |
| 15-A | 4.75 | 100.00 | 0.07 | 1.24 | 0.74 | 5.12 | 0.00 |
| 15-B | 4.94 | 100.00 | 0.08 | 1.23 | 0.72 | 5.02 | 0.00 |
| 15-C | 5.05 | 100.00 | 0.07 | 1.26 | 0.67 | 4.87 | 0.00 |
| 15-D | 4.98 | 100.00 | 0.08 | 1.23 | 0.71 | 4.86 | 0.00 |
| 15-E | 5.21 | 100.00 | 0.08 | 0.90 | 0.99 | 5.31 | 0.00 |
| 15-F | 5.04 | 100.00 | 0.07 | 0.90 | 0.57 | 4.90 | 0.00 |
| 14-B | 9.99 | 100.00 | 0.10 | 1.28 | 0.95 | 5.18 | 0.00 |
| 15-G | 10.49 | 100.00 | 0.07 | 1.05 | 1.04 | 5.37 | 0.00 |
| 15-H | 10.04 | 100.00 | 0.06 | 1.29 | 0.62 | 4.86 | 0.00 |
| 15-I | 9.98 | 100.00 | 0.04 | 0.97 | 1.01 | 5.26 | 0.00 |
| 15-J | 10.19 | 100.00 | 0.07 | 0.96 | 1.00 | 5.40 | 0.00 |
| 15-K | 10.18 | 100.00 | 0.09 | 0.84 | 0.97 | 5.37 | 0.00 |
| 15-L | 10.28 | 100.00 | 0.08 | 0.89 | 0.97 | 5.34 | 0.00 |

### Example 17

In Example 17, the aqueous dronabinol formulations 14-A, 14-B and formulations 15-A through 15-L were stored at 40° C. for one month and analyzed in accordance with Example 16. The concentrations of dronabinol and the impurities/degradants [Cannabidiol (CBD), Cannabinol (CBN) and D8THC] of these formulations are set forth in Table 26 below.

TABLE 26

| | | | | | | Total | |
|---|---|---|---|---|---|---|---|
| | Conc. of | | | | D8- | Impurities | |
| Formulation | THC | Potency | | CBN | THC | (%) | |
| # | (mg/mL) | % | CBD % | % | % | % | % |
| 14-A | 4.95 | 98.68 | 0.04 | 1.29 | 0.81 | 7.45 | 2.54 |
| 15-A | 4.66 | 98.05 | 0.07 | 1.29 | 0.78 | 6.64 | 1.52 |
| 15-B | 4.88 | 98.79 | 0.06 | 1.29 | 0.81 | 6.30 | 1.28 |
| 15-C | 5.02 | 99.30 | 0.05 | 1.28 | 0.78 | 6.31 | 1.44 |
| 15-D | 4.97 | 99.73 | 0.06 | 1.30 | 0.81 | 6.36 | 1.50 |
| 15-E | 4.88 | 93.68 | 0.08 | 1.54 | 0.84 | 10.25 | 4.94 |
| 15-F | 4.79 | 95.10 | 0.06 | 1.77 | 0.76 | 10.51 | 5.61 |
| 14-B | 9.70 | 97.13 | 0.05 | 1.32 | 0.88 | 6.96 | 1.78 |
| 15-G | 10.06 | 95.92 | 0.05 | 1.31 | 0.87 | 7.04 | 1.67 |
| 15-H | 9.89 | 98.50 | 0.05 | 1.31 | 0.88 | 6.83 | 1.97 |
| 15-I | 9.76 | 97.70 | 0.06 | 1.31 | 0.86 | 7.00 | 1.74 |
| 15-J | 9.75 | 95.72 | 0.06 | 1.32 | 0.87 | 7.02 | 1.62 |
| 15-K | 9.81 | 96.36 | 0.07 | 1.51 | 0.78 | 8.65 | 3.28 |
| 15-L | 9.50 | 92.42 | 0.05 | 1.77 | 0.77 | 9.87 | 4.53 |

### Example 18

In Example 18, the aqueous dronabinol formulations 14-A, 14-B and formulations 15-A through 15-L were stored at 40° C. for two months and analyzed in accordance with Example 16. The concentrations of dronabinol and the impurities/degradants [Cannabidiol (CBD), Cannabinol (CBN) and D8THC] of these formulations are set forth in Table 27 below.

TABLE 27

| | | | | | | Total | |
|---|---|---|---|---|---|---|---|
| | Conc. of | | | | | Impurities | |
| Formulation | THC | Potency | CBD | CBN | D8- | (%) | |
| # | (mg/mL) | % | % | % | THC % | % | % |
| 14-A | 4.87 | 97.05 | 0.05 | 1.37 | 0.78 | 7.55 | 2.64 |
| 15-A | 4.57 | 96.18 | 0.10 | 1.34 | 0.82 | 7.95 | 2.83 |
| 15-B | 4.77 | 96.56 | 0.04 | 1.32 | 0.80 | 7.22 | 2.20 |
| 15-C | 4.91 | 97.24 | 0.07 | 1.32 | 0.81 | 7.43 | 2.56 |
| 15-D | 4.87 | 97.77 | 0.06 | 1.34 | 0.82 | 7.58 | 2.72 |
| 15-E | 4.77 | 91.44 | 0.06 | 2.05 | 0.89 | 13.46 | 8.15 |
| 15-F | 4.68 | 92.94 | 0.05 | 2.62 | 0.90 | 13.68 | 8.78 |
| 14-B | 9.69 | 96.98 | 0.06 | 1.34 | 0.83 | 7.73 | 2.55 |
| 15-G | 9.84 | 93.78 | 0.05 | 1.35 | 0.86 | 8.25 | 2.88 |
| 15-H | 9.70 | 96.59 | 0.06 | 1.34 | 0.88 | 8.09 | 3.23 |
| 15-I | 9.65 | 96.65 | 0.06 | 1.35 | 0.90 | 8.56 | 3.30 |
| 15-J | 9.56 | 93.81 | 0.07 | 1.38 | 0.90 | 8.77 | 3.37 |
| 15-K | 9.72 | 95.42 | 0.06 | 1.98 | 0.87 | 11.29 | 5.92 |
| 15-L | 9.39 | 91.34 | 0.05 | 2.46 | 0.88 | 12.21 | 6.87 |

### Example 19

In Example 19 the aqueous dronabinol formulations 14-A, 14-B and formulations 15-A through 15-L were stored at 40° C. for three months and analyzed in accordance with Example 16. The concentrations of dronabinol and the impurities/degradants [Cannabidiol (CBD), Cannabinol (CBN) and D8THC] of these formulations are set forth in Table 28 below.

TABLE 28

| | | | | | | Total | |
|---|---|---|---|---|---|---|---|
| | Conc. of | | | | | Impurities | |
| Formulation | THC | Potency | CBD | CBN | D8- | (%) | |
| # | (mg/mL) | % | % | % | THC % | % | % |
| 14-A | 4.88 | 97.35 | 0.05 | 1.38 | 0.80 | 8.86 | 3.95 |
| 15-A | 4.57 | 96.20 | 0.08 | 1.41 | 0.82 | 9.37 | 4.25 |
| 15-B | 4.93 | 99.72 | 0.06 | 1.38 | 0.81 | 8.72 | 3.70 |
| 15-C | 4.92 | 97.31 | 0.04 | 1.37 | 0.85 | 8.85 | 3.98 |
| 15-D | 4.87 | 97.65 | 0.05 | 1.39 | 0.80 | 8.79 | 3.93 |
| 15-E | 4.73 | 90.77 | 0.05 | 2.58 | 0.88 | 14.98 | 9.67 |
| 15-F | 4.64 | 92.08 | 0.03 | 3.17 | 0.79 | 14.63 | 9.73 |
| 14-B | 9.74 | 97.52 | 0.05 | 1.36 | 0.83 | 8.99 | 3.81 |
| 15-G | 9.90 | 94.42 | 0.04 | 1.40 | 0.84 | 9.61 | 4.24 |
| 15-H | 9.73 | 96.92 | 0.09 | 1.39 | 0.82 | 9.76 | 4.90 |
| 15-I | 9.66 | 96.77 | 0.06 | 1.40 | 0.81 | 9.73 | 4.47 |
| 15-J | 9.62 | 94.37 | 0.07 | 1.44 | 0.79 | 9.81 | 4.41 |
| 15-K | 9.58 | 94.06 | 0.04 | 2.56 | 0.93 | 12.59 | 7.22 |
| 15-L | 9.33 | 90.81 | 0.03 | 3.07 | 0.77 | 13.43 | 8.09 |

The above stability formulations show that formulations with different sugars are as stable as control formulations. As can be seen from the above examples, the aqueous dronabinol formulations with sugars are stable. As far as sugars are concerned, the Delta-9-THC formulations with sucrose are most stable.

### Sublingual Formulations

Sublingual drug delivery is the most preferred method of systemic drug delivery that offers several advantages over both injectable and enteral methods. Because the oral mucosa is highly vascularised, sublingual delivery of drugs results in fast absorption of drugs directly into systemic circulation, bypassing the gastrointestinal tract and first-pass metabolism in the liver. This results in rapid onset of action via a more comfortable and convenient delivery route than the intravenous route. The sublingual formulations will be designed to deliver the drug rapidly into the systemic circulation, providing patients with a noninvasive, easy to use and non-intimidating option with minimal or no side effects.

### Example 20

### Dronabinol Sublingual Droplets Control Formulation

In Example 20, a dronabinol sublingual control formulation was prepared having a concentration of 25 mg/ml. The formulation is listed in Table 29 below:

TABLE 29

| Ingredient | Percent |
|---|---|
| Concentration of | percent to make 25 mg/ml |
| Dronabinol | |
| Absolute Alcohol (ethanol) | 50 |
| % (v) | |
| Propylene glycol % (v) | 50 |

### Example 21

### Dronabinol Sublingual Droplets

In Example 21, a dronabinol sublingual formulation is prepared having a concentration of 6 mg/ml utilizing a phosphate buffer. The formulation is listed in Table 30 below:

TABLE 30

| Ingredient | Percent |
|---|---|
| Concentration of | percent to make 6 mg/ml |
| Dronabinol | |
| Absolute Alcohol (ethanol) | 59 |
| % (v) | |
| Phosphate Buffer (pH 6.50) | 41 |
| % (v) | |

### Example 22

### Dronabinol Sublingual Droplets

In Example 22, a dronabinol sublingual formulation is prepared having a concentration of 6.5 mg/ml and utilizing an ethanolamine citrate buffer (pH 7.01). The formulation is listed in Table 31 below:

TABLE 31

| Ingredient | Percent |
|---|---|
| Concentration of | percent to make 6.5 mg/ml |
| Dronabinol | |
| Absolute Alcohol (ethanol) | 59 |
| % (v) | |
| Ethanolamine Citrate Buffer | 41 |
| (pH 7.01) % (v) | |

### Example 23

### Dronabinol Sublingual Droplets

In Example 23, a dronabinol sublingual formulation is prepared having a concentration of 5 mg/ml and utilizing a phosphate buffer. The formulation is listed in Table 32 below:

TABLE 32

| Ingredient | Percent |
|---|---|
| Concentration of | percent to make 5 mg/ml |
| Dronabinol | |
| Absolute Alcohol (ethanol) | 45 |
| % (v) | |
| Propylene Glycol | 12.5 |
| Polyethylene Glycol | 2.5 |
| Phosphate Buffer (pH 6.5) | 40 |
| % (v) | |

### Example 24

### Dronabinol Sublingual Droplets

In Example 24, a dronabinol sublingual formulation is prepared having a concentration of 10.12 mg/ml and utilizing a phosphate buffer. The formulation is listed in Table 33 below:

TABLE 33

| Ingredient | Percent |
|---|---|
| Concentration of | percent to make 10.12 mg/ml |
| Dronabinol | |
| Absolute Alcohol (ethanol) | 70 |
| % (v) | |
| Mannitol | 1 |
| Phosphate Buffer (pH 7.00) | 30 |
| % (v) | |

### Example 25

### Dronabinol Sublingual Droplets

In Example 25, a dronabinol sublingual formulation is prepared having a concentration of 10.12 mg/ml and utilizing a phosphate buffer. The formulation is listed in Table 34 below:

TABLE 34

| Ingredient | Percent |
|---|---|
| Concentration of | percent to make 10.12 mg/ml |
| Dronabinol | |
| Absolute Alcohol (ethanol) | 67 |
| % (v) | |
| Sodium Lauryl Sulfate | 0.5 |
| Phosphate Buffer (pH 7.00) | 32.5 |
| % (v) | |

### Example 26

### Dronabinol Sublingual Droplets

In Example 26, a dronabinol sublingual formulation is prepared having a concentration of 25 mg/ml. The formulation is listed in Table 35 below:

TABLE 35

| Ingredient | Percent |
|---|---|
| Concentration of | percent to make 25 mg/ml |
| Dronabinol | |
| Absolute Alcohol (ethanol) | 50 |
| % (v) | |
| Propylene Glycol % (v) | 25 |
| Miglyol % (v) | 25 |

### Preparation of Formulations (Examples 20-26)

The formulations of Examples 20-26 are prepared in accordance with the procedure used to prepare the formulations described in Example 1. The sublingual formulations are then prepared by adding the inactive ingredients (e.g., Mannitol and Myglyol), and mixed well.

The final solutions are vortexed for 3 minutes. After mixing, the formulations are stored in refrigerator for further studies.

The formulations are sprayed using a 0.10 ml multidose nasal spray pump by Pfeiffer of America, Princeton, N.J. and the droplets are measured using a Malvern Mastersizer S device, by Malvern Instruments Ltd. A single depression of the sublingual spray pump generates a plume which is then analyzed for spray particles. The sample size for the dose volume, spray pattern, and droplet size distribution is 25 sprays.

### Droplet Volume

In the droplet volume evaluation, 25 spray samples are evaluated using 5 different stroke numbers for each spray sample. Upon testing, results similar to the following would be expected to be measured:

| Overall mean = | 100.4 μl |
|---|---|
| Maximum single actual value = | 103.2 μl |
| Lowest single actual value = | 95.3 μl |
| Standard deviation = | 1.1 |
| Range = | 7.9 |
| Coefficient of variation = | 1.1% |

### Spray Pattern

In the spray pattern evaluation, 25 spray samples are evaluated using a manual actuation at 30 mm from the target. The formulation is dyed with methylene blue and the following spray pattern results are measured. Upon testing, results similar to the following would be expected to be measured:

Small diameter [mm]

| | |
|---|---|
| min | 35.4 |
| mean | 50.6 |
| max | 62 |
| s: | 7.00 |

largest diameter [mm]

| | |
|---|---|
| min | 40 |
| mean | 56.9 |
| max | 67 |
| s: | 6.01 |

spray angle

| | |
|---|---|
| min | 64° |
| mean | 83.3° |
| max | 94° |
| s: | 7.03 |

ratio (largest/smallest diameter)

| | |
|---|---|
| min | 1.04 |
| mean | 1.13 |
| max | 1.33 |
| s | 0.073 |

### Droplet Size Distribution

In the droplet size distribution evaluation, 25 spray samples are evaluated using a manual actuation at 30 mm from the target. The following droplet size distribution results are measured. Upon testing, results similar to the following would be expected to be measured:

Percentage share of droplet diameters at 10 μm [%]

| | |
|---|---|
| min | 0.65 |
| mean | 1.66 |
| max | 2.70 |
| s | 0.527 |

10% of the droplet diameters are smaller than the indicated value [μm]

| | |
|---|---|
| min | 15 |
| mean | 18.2 |
| max | 23 |
| s: | 1.91 |

50% of the droplet diameters are smaller than the indicated value [μm]

| | |
|---|---|
| min | 35 |
| mean | 44.7 |
| max | 65 |
| s: | 7.52 |

90% of the droplet diameters are smaller than the indicated value [μm]

| | |
|---|---|
| min | 96 |
| mean | 154.4 |
| max | 349 |
| s | 64.42 |

### Example 27

### In-Vitro Permeability Testing

In-vitro permeability measurement offers a number of advantages and has been a useful tool to study the mechanisms of oral mucosal drug absorption. Experimentally, a piece of fresh buccal mucosal tissue was mounted to a vertical diffusion cell (Franz cells) to study drug diffusion in a well controlled environment. The buccal mucosal tissues (EpiOral) were supplied by MatTek Corporation. MatTek produces normal, human cell-derived, three dimensional, organotypic in-vitro tissue models that are an alternative to traditional animal testing and is useful as a first order approximation for permeability characteristics of drugs delivered through oral mucosal tissues. The permeability of the drug across the MatTek buccal tissue is measured by the drug concentration on the receiver side, which, by analogy, is equivalent to drug that is available to systemic circulation.

In Example 27, the formulations of Examples 20-26 were studied for in-vitro permeation characteristics. The in-vitro permeation study results of these Examples are listed in Table 36 below.

TABLE 36

| Time | Cumulative amount permeated (ug) | | | | | | |
|---|---|---|---|---|---|---|---|
| (min) | Ex. 20 | Ex. 21 | Ex. 22 | Ex. 23 | Ex. 24 | Ex. 25 | Ex. 26 |
| 0 | 0.00 | 0.00 | 0.00 | 0.00 | 0.00 | 0.00 | 0.00 |
| 5 | 3.42 | 26.55 | 16.59 | 12.80 | 46.70 | 15.22 | 2.66 |
| 10 | 11.60 | 69.22 | 45.54 | 70.29 | 120.96 | 55.66 | 10.53 |
| 15 | 20.76 | 120.89 | 85.89 | 156.76 | 189.91 | 102.33 | 88.52 |
| 30 | 52.35 | 252.08 | 215.78 | 417.76 | 300.46 | 221.61 | 127.01 |
| 45 | 87.00 | 435.83 | 356.44 | 542.93 | 451.44 | 349.90 | 242.16 |
| 60 | 120.32 | 613.37 | 509.21 | 674.44 | 880.77 | 467.78 | 385.91 |
| 90 | 176.68 | 905.81 | 792.76 | 925.01 | 1655.53 | 690.30 | 653.06 |
| 120 | 231.76 | 1197.09 | 1115.63 | 1179.11 | 1853.45 | 922.98 | 917.83 |

Many other variations of the present invention will be apparent to those skilled in the art and are meant to be within the scope of the claims appended hereto.

### Example 28

### Transdermal Gel (Prophetic)

In Example 28, a dronabinol transdermal formulation is prepared having a concentration of 50 mg/ml. The formulation is listed in Table 37 below:

TABLE 37

| DI Water % (v) | QS |
|---|---|
| Ingredient | Percent |
| Concentration of | percent to make 50 mg/ml |
| Dronabinol | |
| Ethanol % (v) | 30 |
| Propylene glycol % (v) | 5 |
| Carbopol | 1.5 |
| NaOH | 1 |
| Isopropyl myristate | 5 |

### Example 29

### Transdermal Gel (Prophetic)

In Example 29, a dronabinol transdermal formulation is prepared having a concentration of 100 mg/ml. The formulation is listed in Table 38 below:

TABLE 38

| Ingredient | Percent |
|---|---|
| Concentration of | percent to make 100 mg/ml |
| Dronabinol | |
| Ethanol % (v) | 45 |
| Propylene glycol % (v) | 10 |
| Carbopol | 2 |
| NaOH | 2.5 |
| Isopropyl myristate | 8 |
| DI Water % (v) | QS |

### Example 30

### Transdermal Gel (Prophetic)

In Example 30, a dronabinol transdermal formulation is prepared having a concentration of 200 mg/ml. The formulation is listed in Table 39 below:

TABLE 39

| Ingredient | Percent |
|---|---|
| Concentration of | percent to make 200 mg/ml |
| Dronabinol Base | |
| Ethanol % (v) | 65 |
| Propylene glycol % (v) | 5 |
| DI Water % (v) | QS |
| Carbopol | 3 |
| NaOH | 5 |
| Isopropyl myristate | 10 |

### Example 31

### Transdermal Formulations (Prophetic)

In Example 31, a dronabinol transdermal formulation is prepared having a concentration of 300 mg/ml. The formulation is listed in Table 40 below:

TABLE 40

| Ingredient | Percent |
|---|---|
| Concentration of | percent to make 300 mg/ml |
| Dronabinol Base | |
| Ethanol % (v) | 65 |
| Propylene glycol % (v) | 5 |
| Buffer % (v) | QS |
| Carbopol | 5 |
| NaOH | 5 |
| Isopropyl myristate | 10 |

Upon stability testing in accordance with the above examples, the results expected are similar to those obtained with the above formulations, to show that the aqueous dronabinol formulations are stable at room temperature for at least 2 years.

### Example 32

### Intravenous Formulations (Prophetic)

In Example 32, aqueous dronabinol formulations for intravenous administration are prepared in accordance with the procedure set forth above in accordance with Examples 1-5 concerning aqueous dronabinol formulations for administration via nebulizer. The dronabinol concentration is adjusted to provide a dose that is therapeutically equivalent to the equivalent oral dosage. The pH and tonicity of the formulations are suitable for intravenous administration. The formulations may also contain pH modifiers and tonicity modifiers. The intravenous formulations are then stored in stoppered glass multi-dose or single dose injection vials. Stability testing and analysis is performed in accordance with Examples 6-14. The stability results expected for the intravenous formulations are similar to those found with the testing performed on the nebulizer formulations. The results are expected to show that the intravenous formulations are stable for at least 2 years at room temperature.

### Example 33

### Dronabinol Ophthalmic Formulations

Dronabinol 1% (w/w) ophthalmic formulations 33-A through 33-E were prepared. The formulations contained additional ingredients as set forth in Table 41 below.

TABLE 41

| | Conc. of | | | Mineral | |
|---|---|---|---|---|---|
| Formulation | THC | Lanolin | Petrolatum | Oil | Water |
| # | (% w/w) | (% w/w) | (% w/w) | (% w/w) | (% w/w) |
| 33-A | 1 | — | 100 | — | — |
| 33-B | 1 | 100 | — | — | — |
| 33-C | 1 | 25 | 75 | — | — |
| 33-D | 1 | 25 | 50 | 25 | — |
| 33-E | 1 | 20 | 50 | 10 | 20 |

Delta-9-tetrahydrocannabinol is chemically synthesized as per procedures known to those skilled in the art, and is supplied as a light-yellow resinous oil that is sticky at room temperature and hardens upon refrigeration. Chemically synthesized dronabinol is supplied in a round bottom flask with high-vacuum adaptor with a 24/40 o-ring seal joint and bakeable PTFE plug.

Dronabinol 1% (w/w) ophthalmic formulations 33-A through 33-E were then prepared as follows: An oil bath (vacuum pump oil, Fisher CAS #72623-87-1) was heated to 90-95° C. A container containing the delta-9-THC was placed in the preheated oil bath for 10 min (after removing the vacuum adapter of the container containing D9-THC) until it turns into a flowable liquid. The weight of the empty glass container (W₁) was calculated. The D9-THC was transferred to the container by using a glass pipette.

The weight of the container with Delta-9-THC (W₂) was calculated. The exact amount of D9-THC transferred was calculated according to the following formula: (ΔW<W₂−W₁). The actual amount of D9-THC in the container was calculated according to the following formula: D9-THC=ΔW×Potency.

The additional ingredients in accordance with Table 41 for formulations 33-A through 33-E were then added. The mixtures were then heated until the contents became a flowable liquid. The mixtures were then mixed well by a vortexer for about 3 minutes while still hot. The formulations were then allowed to return to room temperature before storage.

### CONCLUSION

Many other variations of the present invention will be apparent to those skilled in the art and are meant to be within the scope of the claims appended hereto. The foregoing specification alludes to beliefs, hypothesis and conclusions of the inventor based on his experience in the field, the reports of others (such as those identified in the publications identified herein), and experiments conducted and reported herein, and are provided for purposes of (possible) explanation only and are not meant to limit the invention in any manner whatsoever.

### REFERENCES

Armstrong N A, James K C, Pugh W K L. Drug migration into soft gelatin capsule shells and its effect on in-vitro availability. J. Pharm. Pharmacol. 36: 361-365, 1984
Bauer K H. Die herstellung von hart- und weichgelatinekapseln. In: Die Kapsel. Stuttgart: Wissenschaftliche Verlags GmbH. Editors: Fahrig W, Hofer U H, 58-82, 1983.
Beckstrom-Stenberg S M and Duke J A. “The phytochemical database.” Ars-genome.cornell.edu/cgi-bin/WebAce/webace?db=phytochemdb. (Data version July 1994).
Bradley Morris J. Food, industrial, nutraceutical, and pharmaceutical uses of sesame genetic resources. In: Trends in New Crops and New Uses. Editors: Janick J and Whipkey A, 2002.
Cade D, Cole E T, Mayer J-Ph, Wittwer F. Liquid filled and sealed hard gelatin capsules. Acta Pharm. Technol. 29: 245-251, 1983.
Ewart T. Cole. Liquid-filled and sealed hard gelatin capsule technologies. In: Modified-Release Drug Delivery Technology. Editors: Rathbone M J, Hadgraft J, Roberts M S, Publishers Marcel Dekker, 2002.
Featured Excipients Antioxidants. Int. J. Pharm. Compounding. 3(1): 52-, January/February 1999.
Hom F S, Veresh S A, Ebert W R. Soft gelatin capsules. II. Oxygen permeability study of capsule shells. J. Pharm. Sci. 64(5): 851-857, 1975.
Kato M J, Chu A, Davin L B. Lewis N G, Biosynthesis of antioxidant lignans in sesamum indicum seeds. Phytochemistry. 47: 583-591, 1998.
Martin A, Bustamante P, and Chun A H C. Physical Pharmacy. Fourth ed., Lea & Febiger, 1993.
Mechoulam R. Chemistry of cannabis. Handbook Exp. Pharmacol. 55: 119-134, 1981.
Physicians Desk Reference®, ed. 2003.
Razdan, Raj, K., Structure-Activity Relationships in Cannabinoids. Pharmacological Reviews, 38(2): 75-149, 1986.
Shah N H, Phuarpradit W, Ahmed H. Liquid filling in hard gelatin capsules: formulations and processing considerations. American Pharmaceutical Review. 6(1): 14-21, Spring 2003.
Sirato-Yasumoto S, Katsuta M, Okuyama Y, Takahashi Y, and Ide T. Effect of sesame seeds rich in sesamin and sesamolin on fatty acid oxidation in rat liver. J. Agr. Food Chem. 49: 2647-2651, 2001.
U.S. Department of Health and Human Services, Food and Drug Administration “Guidance for Industry: Q1A (R2) Stability Testing of New Drug Substances and Products.” ICH, November 2003.

All of the above references (patents and non-patent publications) are hereby incorporated by reference.

1. A stabilized cannabinoid formulation, comprising an effective amount of a cannabinoid in a semi-aqueous solution buffered to a pH of from about 5 to about 10, the solution comprising water and an effective amount of an organic cosolvent to maintain the physical stability of the formulation, the formulation containing at least about 80% of the amount of cannabinoid in undegraded form after exposure of the formulation to a storage condition selected from the group consisting of (i) 40° C./60% relative humidity for 1 month; (ii) 40° C./60% relative humidity for 2 months; (iii) 40° C./60% relative humidity for 3 months; (iv) 40° C./60% relative humidity for 6 months; (v) 40° C./60% relative humidity for 8 months; (vi) room temperature (25° C.)/60% relative humidity for one year; (vii) room temperature (25° C.)/60% relative humidity for two years; and any combination thereof.

2. A stabilized cannabinoid formulation, comprising an effective amount of a cannabinoid in a semi-aqueous solution buffered to a pH of from about 5 to about 10, said solution comprising from about 20% to about 44% water and an effective amount of an organic cosolvent to maintain the physical stability of the formulation such that the formulation is not cloudy and has no visible oil droplets, the formulation containing at least about 80% of the amount of cannabinoid in undegraded form after exposure of the formulation to a storage condition selected from the group consisting of (i) 40° C./60% relative humidity for 1 month; (ii) 40° C./60% relative humidity for 2 months; (iii) 40° C./60% relative humidity for 3 months; (iv) 40° C./60% relative humidity for 6 months; (v) 40° C./60% relative humidity for 8 months; (vi) room temperature (25° C.)/60% relative humidity for one year; (vii) room temperature (25° C.)/60% relative humidity for two years; and any combination thereof.

3. A stabilized cannabinoid formulation, comprising an effective amount of a cannabinoid in a semi-aqueous solution buffered to a pH of from about 5 to about 10, said solution comprising water in an amount greater than 30% to about 44% of the formulation, and an effective amount of an organic cosolvent to maintain the physical stability of the formulation, the formulation containing at least about 80% w/w of the amount of cannabinoid in undegraded form after exposure of the formulation to a storage condition selected from the group consisting of (i) 40° C./60% relative humidity for 1 month; (ii) 40° C./60% relative humidity for 2 months; (iii) 40° C./60% relative humidity for 3 months; (iv) 40° C./60% relative humidity for 6 months; (v) 40° C./60% relative humidity for 8 months; (vi) room temperature (25° C.)/60% relative humidity for one year; (vii) room temperature (25° C.)/60% relative humidity for two years; and any combination thereof.

4. The stabilized cannabinoid formulation of claim 1, wherein the amount of water is about 35%.

5. The formulation of claim 1, further comprising a second cosolvent selected from the group consisting of ethanol, propanol, propylene glycol, polyethylene glycol, labrosol, labrafil, transcutol and combinations thereof.

6. The formulation of claim 1, wherein the cannabinoid is selected from the group consisting of dronabinol, 11-OH-delta-9-THC, delta-8-THC, 11-OH-delta-8-THC, nabilone, levonantradol, (−)-HU-210, Win 55212-2, Anandamide, Methandamide, CP 55940, O-1057 and SR141716A.

7. The formulation of claim 6, wherein the cannabinoid is dronabinol.

8. The formulation of claim 7, wherein dronabinol is present in a concentration of from about 0.001 to about 500 mg/ml.

9. The formulation of claim 7, wherein dronabinol is present in a concentration of from about 0.05 mg/ml to about 100 mg/ml.

10. The formulation of claim 7, wherein dronabinol is present in a concentration of from about 0.5 mg/ml to about 10 mg/ml.

11. The formulation of claim 7, wherein dronabinol is present in a concentration of from about 1 to about 10 mg/ml.

12. The formulation of claim 7, wherein dronabinol is present in a concentration of about 2 mg/ml to about 5 mg/ml.

13. The formulation of claim 1 comprising the following volumetric amounts: from about 15 to about 70% ethanol and from about 5 to about 40% of a glycol.

14. The formulation of claim 1, wherein said organic solvent comprises from about 35 to about 45% ethanol, from about 5 to about 15% PEG-400, and from about 5 to about 15% propylene glycol.

15. The formulation of claim 1, which contains at least about 80% of the amount of cannabinoid in undegraded form after exposure of the formulation to storage condition of 40° C./60% relative humidity for 1 month.

16. The formulation of claim 1, which contains at least about 80% of the amount of cannabinoid in undegraded form after exposure of the formulation to storage condition of 40° C./60% relative humidity for 2 months.

17. The formulation of claim 1, which contains at least about 80% of the amount of cannabinoid in undegraded form after exposure of the formulation to storage condition of 40° C./60% relative humidity for 3 months.

18. The formulation of claim 1, which contains at least about 80% of the amount of cannabinoid in undegraded form after exposure of the formulation to storage condition of 40° C./60% relative humidity for 6 months.

19. The formulation of claim 1, which contains at least about 80% of the amount of cannabinoid in undegraded form after exposure of the formulation to storage condition of 40° C./60% relative humidity for 8 months.

20. The formulation of claim 1, which contains at least about 80% of the amount of cannabinoid in undegraded form after exposure of the formulation to storage condition of room temperature (25° C.)/60% relative humidity for one year.

21. The formulation of claim 1, which contains at least about 80% of the amount of cannabinoid in undegraded form after exposure of the formulation to storage condition of room temperature (25° C.)/60% relative humidity for two years.

22. The formulation of claim 1, which contains at least about 90% of the amount of cannabinoid in undegraded form after exposure of the formulation to storage condition selected from the group consisting of (i) 40° C./60% relative humidity for 1 month; (ii) 40° C./60% relative humidity for 2 months; (iii) 40° C./60% relative humidity for 3 months; (iv) 40° C./60% relative humidity for 6 months; (v) 40° C./60% relative humidity for 8 months; (vi) room temperature (25° C.)/60% relative humidity for one year; (vii) room temperature (25° C.)/60% relative humidity for two years; and any combination thereof.

23. The formulation of claim 1, which contains at least about 90% of the amount of cannabinoid in undegraded form after exposure of the formulation to storage condition of 40° C./60% relative humidity for 1 month.

24. The formulation of claim 1, which contains at least about 90% of the amount of cannabinoid in undegraded form after exposure of the formulation to storage condition of 40° C./60% relative humidity for 2 months.

25. The formulation of claim 1, which contains at least about 90% of the amount of cannabinoid in undegraded form after exposure of the formulation to storage condition of 40° C./60% relative humidity for 3 months.

26. The formulation of claim 1, which contains at least about 90% of the amount of cannabinoid in undegraded form after exposure of the formulation to storage condition of 40° C./60% relative humidity for 6 months.

27. The formulation of claim 1, which contains at least about 90% of the amount of cannabinoid in undegraded form after exposure of the formulation to storage condition of 40° C./60% relative humidity for 8 months.

28. The formulation of claim 1, which contains at least about 90% of the amount of cannabinoid in undegraded form after exposure of the formulation to storage condition of room temperature (25° C.)/60% relative humidity for one year.

29. The formulation of claim 1, which contains at least about 90% w/w of the cannabinoid in undegraded form after exposure of the formulation to storage condition of room temperature (25° C.)/60% relative humidity for two years.

30. The formulation of claim 1 further comprising a stabilizing agent.

31. The formulation of claim 30 further comprising an effective amount of one or more stabilizers to promote stability of the cannabinoid against unacceptable degradation.

32. The formulation of claim 30 further comprising an effective amount of one or more stabilizers to promote stability of the cannabinoid against unacceptable degradation, said stabilizers selected from the group consisting of organic bases, inorganic bases and antioxidants.

33. The formulation of claim 32 further comprising an effective amount of one or more organic bases to promote stability of the cannabinoid against unacceptable degradation.

34. The formulation of claim 32 further comprising an effective amount of one or more inorganic bases to promote stability of the cannabinoid against unacceptable degradation.

35. The formulation of claim 32 further comprising an effective amount of one or more antioxidants to promote stability of the cannabinoid against unacceptable degradation.

36. The formulation of claim 30, wherein said stabilizing agent is selected from the group consisting of ascorbic acid, cysteine hydrochloride, sodium bisulfate, sodium meta bisulfate, sodium sulfate, ascorbyl palmitate, butylated hydroxyanisole, butylated hydroxytoluene, lecithin, propyl gallate, alpha-tocopherol, citric acid, ethylenediamine tetraacetic acid, sorbitol, tartraric acid, phosphoric acid, povidone and combinations thereof.

37. The formulation of claim 30, wherein said stabilizing agent is selected from the group consisting of butylated hydroxyanisole, butylated hydroxytoluene and sodium ascorbate.

38. The formulation of claim 30, wherein said stabilizing agent is selected from the group consisting of methanolamine and ascorbic palmitate.

39. The formulation of claim 1, wherein the cannabinoid is dronabinol, and the formulation does not contain during its labeled shelf-life, unacceptable levels of dronabinol degradants selected from the group consisting of greater than 2% delta-8 tetrahydrocannabinol (D8THC), greater than 2% cannabinol (CBN), greater than 2% cannabidiol (CBD), and any combination thereof.

40. The formulation of claim 1 which further comprises one or more additional therapeutically active agents.

41. The formulation of claim 40, wherein said additional therapeutically active agent is selected from a narcotic analgesic, a non-narcotic analgesic, an anti-emetic, a steroid, and mixtures of any of the foregoing.

42. The formulation of claim 1 wherein the cannabinoid is dronabinol in an amount from about 0.01 mg to about 500 mg per dose.

43. The formulation of claim 1 wherein the cannabinoid is dronabinol in an amount from about 0.5 mg to about 200 mg per dose.

44. The formulation of claim 1 wherein the cannabinoid is dronabinol in an amount from about 1 mg to about 500 mg per dose.

45. The formulation of claim 1 wherein the cannabinoid is dronabinol in an amount from about 0.01 mg to about 50 mg per dose.

46. The formulation of claim 1 wherein the cannabinoid is dronabinol in an amount from about 0.01 mg to about 1 mg per dose.

47. The formulation of claim 1, wherein the cannabinoid is dronabinol in an amount from about 0.5 to about 5 mg per dose.

48. The formulation of claim 1, wherein the cannabinoid is dronabinol in an amount from about 1 mg to about 3 mg per dose.

49. The formulation of claim 1, which further comprises an emulsifier.

50. The formulation of claim 1 further comprises a surfactant.

51. The formulation of claim 1, wherein the carrier comprises a buffer comprising a phosphate buffer.

52. The formulation of claim 1, wherein the cannabinoid is in a form selected from the group consisting of its free form, a salt, an ester, a prodrug, a complex, and a mixture of any of the foregoing.

53. The formulation of claim 1, wherein said formulation is homogeneous and thermodynamically stable.

54. The formulation of claim 1, further comprising a preservative.

55. The formulation of claim 1 further comprising an emulsifying agent selected from the group consisting of 44/14; Gelucire 50/13; Imwitor 91; Imwitor 308; Imwitor 380; Imwitor 742; Imwitor 780K; Imwitor 928; Imwitor 988; poloxamer 124; poloxamer 188; Tagat TO; Tween 80; lecithin; lysolecithin; phosphatidylcholine; phosphatidylethanolamine; phosphatidylglycerol; phosphatidic acid; phosphatidylserine; lysophosphatidylcholine; lysophosphatidylethanolamine; lysophosphatidylglycerol; lysophosphatidic acid; lysophosphatidylserine; PEG-phosphatidylethanolamine; PVP-phosphatidylethanolamine; polyoxyethyelene caster oil, cremophor, and combinations thereof.

56. The formulation of claim 1, wherein said carrier comprises components in the following amounts based on weight: from about 45 to about 65% ethanol, from about 4 to about 6% propylene glycol and from about 28 to about 48% water.

57. The formulation of claim 1 wherein the cannabinoid is dronabinol derived from a source selected from the group consisting of a natural source, a synthetic source, a semi-synthetic source, and a mixture of any of the foregoing.

58. The formulation of claim 1, wherein the cannabinoid is dronabinol in a form selected from the group consisting of its free form, a salt, an ester, a prodrug, a complex, and a mixture of any of the foregoing.

59. The formulation of claim 1 comprising the following volumetric amounts: (i) from about 15 to about 50% ethanol, and (ii) a glycol selected from the group consisting of (a) propylene glycol from about 0.1% to about 25%, (b) polyethylene glycol from about 1 to about 30%, and (c) a combination of (a) and (b); said formulation is suitable for administration via a nebulizer.

60. The formulation of claim 1 comprising the following volumetric amounts: (i) from about 35 to about 40% ethanol, (ii) about 10% propylene glycol, (iii) from about 13 to about 16.7% polyethylene glycol and (iv) from about 0.01 to about 0.5% of an antioxidant selected from the group consisting of ascorbic palmitate, BHA, BHT, propyl gallate, sodium ascorbate, tocopherol and monethanolamine; said formulation is suitable for administration via a nebulizer.

61. The formulation of claim 59 that are administered into the lung as aerosolized particles having a mean mass median aerodynamic diameter in the range of from about 0.01 to about 15 microns.

62. The formulation of claim 61 wherein the aerosolized particles have a mean mass median aerodynamic diameter in the range of from about 1 to about 10 microns.

63. The formulation of claim 61 wherein the aerosolized particles have a mean mass median aerodynamic diameter in the range of from about 2 to about 4 microns.

64. A method of controlling nausea and vomiting associated with a human receiving chemotherapy comprising the intrapulmonary administration of a liquid nebulizer formulation to a human patient experiencing nausea and vomiting, said liquid nebulizer formulation comprising an effective amount of a cannabinoid dispersed in a pharmaceutically acceptable carrier comprising at least about 20% water, said carrier buffered to a pH range from about 6.5 to about 7.5.

65. A method of appetite stimulation of an AIDS patient suffering from wasting syndrome comprising the intrapulmonary administration of a liquid nebulizer formulation to a human patient experiencing a lack of appetite, said liquid nebulizer formulation comprising an effective amount of a cannabinoid dispersed in a pharmaceutically acceptable carrier comprising at least about 20% water, said carrier buffered to a pH range from about 6.5 to about 7.5.

66. The method of claim 64, wherein the cannabinoid is dronabinol.

67. The formulation of claim 1 comprising the following volumetric amounts: (i) from about 15 to about 65% ethanol and (ii) a glycol selected from the group consisting of (a) propylene glycol from about 0.1% to about 25%, (b) polyethylene glycol from about 1 to about 25%, and (c) a combination of (a) and (b); said formulation being suitable for oral administration.

68. The formulation of claim 67 further comprising a viscosity modifying agent selected from the group consisting of Aerosil (silicon dioxide); cetostearyl alcohol; cetyl alcohol; stearyl alcohol; Gelucire 33/01; Gelucire 39/01; Gelucire 43/01; glyceryl behenate (Compritol 888 ATO); glyceryl palmitostearate (Precirol AT05); Softisan 100; Softisan 142; Softisan 378; Softisan 649; hydroxypropyl cellulose and mixtures thereof.

69. The formulation of claim 68 wherein the viscosity modifying agent is hydroxypropyl cellulose in an amount of from about 0.1 to about 25%.

70. The formulation of claim 67 further comprising ingredients selected from the group consisting of a pharmaceutically acceptable sweetener, flavoring agent, taste-masking agent, and combinations thereof.

71. The formulation of claim 70 wherein the sweetener is selected from the group consisting of sucrose, fructose, sorbitol, aspartame, acesulfame potassium, sucralose, saccharin, saccharin sodium, xylitol and combinations thereof.

72. The formulation of claim 70, wherein the sweetener is selected from the group consisting of xylitol in an amount of from about 5 to about 25%, saccharin in an amount of from about 0.01 to about 5%, and sodium saccharin in an amount of from about 0.01 to about 5% by weight.

73. The formulation of claim 70 wherein the flavoring agent is selected from the group consisting of peppermint, methyl salicylate, mannitol and fruit flavoring.

74. A method of controlling nausea and vomiting associated with a human receiving chemotherapy comprising orally administering a liquid syrup formulation to a human patient experiencing nausea and vomiting, said liquid syrup formulation comprising an effective amount of a cannabinoid dispersed in a pharmaceutically acceptable carrier comprising at least about 20% water, said carrier buffered to a pH range from about 6.5 to about 7.5.

75. A method of appetite stimulation of an AIDS patient suffering from wasting syndrome comprising orally administering a liquid syrup formulation to a human patient experiencing a lack of appetite, said liquid syrup formulation comprising an effective amount of a cannabinoid dispersed in a pharmaceutically acceptable carrier comprising at least about 20% water, said carrier buffered to a pH range from about 6.5 to about 7.5.

76. The method of claim 74, wherein the cannabinoid is dronabinol.

77. The formulation of claim 1 in the form of discrete liquid droplets comprising the following volumetric amounts: (i) from about 15 to about 70% ethanol and (ii) a glycol selected from the group consisting of (a) propylene glycol from about 0.1% to about 25%, (b) polyethylene glycol from about 1 to about 25%, and (c) a combination of (a) and (b); said formulation being suitable for sublingual administration.

78. The formulation of claim 1 in the form of discrete liquid droplets comprising the following volumetric amounts: (i) from about 45 to about 70% ethanol and (ii) a glycol selected from the group consisting of (a) propylene glycol from 0 to about 50%, (b) polyethylene glycol from 0 to about 2.5%, and (c) a combination of (a) and (b); (iii) a further solubilizing agent from 0 to about 25%; and (iv) a taste masking agent from 0 to about 1%; said formulation being suitable for sublingual administration.

79. The formulation of claim 77, where said droplets having a mean diameter of at least about 10 microns.

80. The sublingual cannabinoid formulation of claim 77, wherein said liquid droplets have a mean diameter of at least about 20 microns.

81. The sublingual cannabinoid formulation of claim 77, wherein said liquid droplets have a size distribution of from about 5 microns to about 500 microns.

82. The sublingual cannabinoid formulation of claim 77, wherein said liquid droplets have a size distribution of from about 10 microns to about 200 microns.

83. The sublingual cannabinoid formulation of claim 77, wherein the cannabinoid is dronabinol included in said formulation in a concentration of from about 0.01 mg/ml to about 10 mg/ml.

84. The sublingual cannabinoid formulation of claim 77, further comprising an absorption enhancer.

85. The sublingual cannabinoid formulation of claim 84, wherein said absorption enhancer is isopropyl myristate.

86. The sublingual cannabinoid formulation of claim 84, wherein said absorption enhancer is in an amount of from about 0.001% to about 10% by weight of the formulation.

87. The sublingual cannabinoid formulation of claim 77, wherein the cannabinoid does not or substantially does not enter the lungs of a human patient after sublingual administration.

88. A unit dose of a sublingual cannabinoid formulation of claim 1 comprising discrete liquid droplets of an effective amount of cannabinoid in a pharmaceutically acceptable liquid carrier suitable for sublingual spray administration; said droplets having a mean diameter of at least about 10 microns.

89. The unit dose of claim 88, wherein said liquid spray formulation comprises droplet particles having a mean diameter of at least about 20 microns.

90. The unit dose of claim 88, wherein said liquid spray formulation comprises droplet particles having a size distribution of from about 5 microns to about 500 microns.

91. The unit dose of claim 88, wherein said liquid spray formulation comprises droplet particles having a size distribution of from about 10 microns to about 200 microns.

92. The unit dose of claim 88, which comprises from about 0.5 mg to about 200 mg of said cannabinoid, pharmaceutically acceptable salt thereof, or derivative thereof.

93. The unit dose of claim 88, which comprises from about 1 mg to about 100 mg of said cannabinoid, pharmaceutically acceptable salt thereof, or derivative thereof.

94. The unit dose of claim 88, which comprises from about 5 mg to about 50 mg of said cannabinoid.

95. A method of controlling nausea and vomiting associated with a human receiving chemotherapy comprising sublingually administering a liquid spray formulation in the form of discrete liquid droplets having a mean diameter of at least about 10 microns to a human patient experiencing nausea and vomiting, said liquid spray formulation comprising an effective amount of a cannabinoid dispersed in a pharmaceutically acceptable liquid carrier comprising at least about 20% water, said carrier buffered to a pH of about 7.

96. A method of appetite stimulation of an AIDS patient suffering from wasting syndrome comprising sublingually administering a liquid spray formulation in the form of discrete liquid droplets having a mean diameter of at least about 10 microns to a human patient experiencing a lack of appetite, said liquid spray formulation comprising an effective amount of a cannabinoid dispersed in a pharmaceutically acceptable liquid carrier comprising at least about 20% water, said carrier buffered to a pH of about 7.

97. The method of claim 95, wherein said cannabinoid is dronabinol.

98. The method of claim 95, wherein said liquid droplets have a mean diameter of at least about 20 microns.

99. The method of claim 95, wherein said discrete liquid droplets have a size distribution of from about 5 microns to about 500 microns.

100. The method of claim 95, wherein said size distribution is from about 10 microns to about 200 microns.

101. The method of claim 1, wherein said cannabinoid is included in said liquid spray formulation in a concentration of from about 1 mg/ml to about 50 mg/ml.

102. The method of claim 95 wherein said cannabinoid is included in said liquid spray formulation in a concentration of from about 5 mg/ml to about 25 mg/ml.

103. The method of claim 95 wherein said cannabinoid is included in said liquid spray formulation in a concentration of from about 6 mg/ml to about 10.12 mg/ml.

104. The method of claim 95 wherein said cannabinoid is included in said liquid spray formulation in a concentration of about 6.5 mg/ml.

105. A unit dose or bi-dose device for sublingual administration of a drug comprising:
a reservoir containing a unit dose or a bi-dose of a liquid formulation comprising an effective amount of a cannabinoid selected from the group consisting of dronabinol, 11-OH-delta-9-THC, delta-8-THC, and 111-OH-delta-8-THC, said cannabinoid in a pharmaceutically acceptable liquid carrier comprising at least about 20% water, said carrier buffered to a pH of about 7; and the device having an actuator which when actuated delivers the unit dose of the liquid formulation in the form of liquid droplets having a mean diameter of at least about 10 microns.

106. A unit dose or bi-dose device for sublingual administration of a drug comprising:
a reservoir containing a unit dose or a bi-dose of a room temperature stable liquid formulation comprising an effective amount of dronabinol in a pharmaceutically acceptable liquid carrier comprising at least about 20% water, said carrier buffered to a pH of about 7; and the device having an actuator which when actuated delivers the unit dose of the liquid formulation in the form of liquid droplets having a mean diameter of at least about 10 microns.

107. The unit dose or bi-dose device of claim 105, wherein said delivered unit dose comprises from about 0.5 mg to about 200 mg of cannabinoid.

108. The unit dose or bi-dose device of claim 105, wherein said delivered unit dose comprises from about 1 mg to about 100 mg of cannabinoid.

109. The unit dose or bi-dose device of claim 105, wherein said delivered unit dose comprises from about 5 mg to about 50 mg of cannabinoid.

110. The unit dose or bi-dose device of claim 105, the device further comprising a stopper comprising a material that precludes or substantially precludes the absorption of the cannabinoid.

111. The unit dose or bi-dose device of claim 105, wherein the stopper is a component of a primary packaging of the device which affects spray characteristics of the liquid formulation.

112. The unit dose or bi-dose device of claim 105, wherein said stopper has the following composition and characteristic: a) elastomer: bromobutyl and/or chlorobutyl; b) reinforcement: inert material: inert mineral; and c) curing system: unconventional.

113. A multi-dose device for sublingual administration of a drug comprising:
a reservoir containing a liquid formulation comprising a cannabinoid selected from the group consisting of dronabinol, 11-OH-delta-9-THC, delta-8-THC, and 11-OH-delta-8-THC, said cannabinoid in a pharmaceutically acceptable liquid carrier comprising at least about 20% water, said carrier buffered to a pH of about 7; and the device having an actuator which when actuated delivers a therapeutically effective dose of the liquid formulation in the form of liquid droplets having a mean diameter of at least about 10 microns.

114. A multi-dose device for sublingual administration of a drug comprising:
a reservoir containing a room temperature stable liquid formulation comprising dronabinol in a pharmaceutically acceptable liquid carrier comprising at least about 20% water, said carrier buffered to a pH of about 7; and
the device having an actuator which when actuated delivers a therapeutically effective dose of the liquid formulation in the form of liquid droplets having a mean diameter of at least about 10 microns.

115. The multi-dose device of claim 113, wherein said therapeutically effective dose comprises from about 0.5 mg to about 200 mg cannabinoid.

116. The multi-dose device of claim 113, wherein said therapeutically effective dose comprises from about 1 mg to about 100 mg cannabinoid.

117. The multi-dose device of claim 113, wherein said therapeutically effective dose comprises from about 5 mg to about 50 mg cannabinoid.

118. The multi-dose device of claim 113, wherein the device further comprises a gasket comprising a material which precludes or substantially precludes the absorption of the cannabinoid.

119. The device of claim 113, wherein said gasket has the following composition and characteristic: a) elastomer: bromobutyl and/or chlorobutyl; b) reinforcement: inert material: inert mineral; and c) curing system: unconventional.

120. The formulation of claim 1 comprising the following volumetric amounts: (i) from about 15% to about 90% ethanol, (ii) a glycol selected from the group consisting of (a) propylene glycol from about 0.1% to about 25%, (b) polyethylene glycol from about 1 to about 30%, and (c) a combination of (a) and (b), (iii) from about 0.1 to about 20% of a gelling agent, (iv) from about 0.1 to about 20% of a base and (v) from about 0.1 to about 20% of an absorption enhancer, said formulation being suitable for transdermal administration.

121. A method of controlling nausea and vomiting associated with a human receiving chemotherapy comprising transdermally administering a gel formulation to a human patient experiencing nausea and vomiting, said gel formulation comprising an effective amount of a cannabinoid dispersed in a pharmaceutically acceptable gel carrier comprising at least about 20% water, said carrier buffered to a pH of about 7.

122. A method of appetite stimulation of an AIDS patient suffering from wasting syndrome comprising transdermally administering a gel formulation to a human patient experiencing a lack of appetite, said gel formulation comprising an effective amount of a cannabinoid dispersed in a pharmaceutically acceptable gel carrier comprising at least about 20% water, said carrier buffered to a pH of about 7.

123. The method of claim 121, wherein said cannabinoid is dronabinol.

124. The formulation of claim 1 comprising the following volumetric amounts: (i) from about 15% to about 90% ethanol, (ii) a glycol selected from the group consisting of (a) propylene glycol from about 0.1% to about 25%, (b) polyethylene glycol from about 1 to about 30%, and (c) a combination of (a) and (b), (iii) from about 0.1 to about 20% of a gelling agent, (iv) from 0 to about 20% of a pH modifying agent and (v) from about 0 to about 20% of tonicity modifying agent, said formulation being suitable for intravenous administration.

125. The formulation of claim 124 wherein said tonicity modifying agent is selected from the group consisting of a sugar, polyalcohol, mannitol, sorbitol, xylitol, sucrose, lactose, sodium chloride, and combinations thereof.

126. A method of controlling nausea and vomiting associated with a human receiving chemotherapy comprising intravenously administering an intravenous formulation to a human patient experiencing nausea and vomiting, said intravenous formulation comprising an effective amount of a cannabinoid dispersed in a pharmaceutically acceptable liquid carrier comprising at least about 20% water, said carrier buffered to a pH of about 7.

127. A method of appetite stimulation of an AIDS patient suffering from wasting syndrome comprising intravenously administering an intravenous formulation to a human patient experiencing a lack of appetite, said intravenous formulation comprising an effective amount of a cannabinoid dispersed in a pharmaceutically acceptable liquid carrier comprising at least about 20% water, said carrier buffered to a pH of about 7.

128. The method of claim 126, wherein said cannabinoid is dronabinol.

129. A stabilized ophthalmic formulation comprising an effective amount of cannabinoid dispersed in a pharmaceutically acceptable carrier, said carrier comprising lanolin, petrolatum or combinations thereof, said formulation containing at least about 80% of the amount of cannabinoid in undegraded form after exposure of the formulation to a storage condition selected from the group consisting of (i) 40° C./60% relative humidity for 1 month; (ii) 40° C./60% relative humidity for 2 months; (iii) 40° C./60% relative humidity for 3 months; (iv) 40° C./60% relative humidity for 6 months; (v) 40° C./60% relative humidity for 8 months; (vi) room temperature (25° C.)/60% relative humidity for one year; (vii) room temperature (25° C.)/60% relative humidity for two years; and any combination thereof.

130. The ophthalmic formulation of claim 129 further comprising mineral oil, water, or combinations thereof.

131. The formulation of claim 129, wherein said carrier comprises from about 20 to about 100% by weight lanolin.

132. The formulation of claim 129, wherein said carrier comprises from about 50 to about 100% by weight petrolatum.

133. The formulation of claims 130, wherein said carrier comprises from about 10 to about 25% by weight mineral oil.

134. The formulation of claims 130, wherein said carrier comprises from about 0.1 to about 20% by weight water.

135. A method of treating a human patient with glaucoma comprising the ophthalmic administration of a room temperature stable ophthalmic formulation to a human patient with glaucoma, said ophthalmic formulation comprising an effective amount of a cannabinoid dispersed in a pharmaceutically acceptable carrier selected from the group consisting of lanolin, petrolatum, and combinations thereof.

136. The method of claim 135 wherein the cannabinoid is dronabinol.

137. A method of treating a human patient experiencing a condition selected from the group consisting of: anorexia associated with AIDS; nausea and vomiting associated with chemotherapy; glaucoma; multiple sclerosis and pain; said method comprising the step of administering to said patient a stabilized cannabinoid formulation, comprising a cannabinoid in an effective concentration, a carrier comprising at least about 20% water, said carrier buffered to a pH range from about 6.5 to about 7.5.

138. The method of claim 137 wherein said formulation is suitable for administration by the delivery route selected from the group consisting of: pulmonary, oral, sublingual, transdermal, intravenous and ophthalmic.
